(19) United States
(12) Patent Application Publication
(10) Pub. No.: US 2024/0182890 A1
(43) Pub. Date: Jun. 6, 2024

(54) COMPOSITIONS AND METHODS FOR SITE-SPECIFIC MODIFICATION

(71) Applicant: ASTRAZENECA AB, SODERTALJE (SE)

(72) Inventors: SONGYUAN LI, SODERTALJE (SE); MARCELLO MARESCA, SODERTALJE (SE); SASA SVIKOVIC, SODERTALJE (SE)

(21) Appl. No.: 18/554,014

(22) PCT Filed: Apr. 6, 2022
(86) PCT No.: PCT/EP2022/059070

§ 371 Date: Oct. 5, 2023

Related U.S. Application Data
(60) Provisional application No. 63/171,651, filed on Apr. 7, 2021.
(60) Provisional application No. 63/292,144, filed on Dec. 21, 2021.

(51) Int. Cl. C12N 15/11; C12N 9/00; C12N 9/22; C12N 15/90

(52) U.S. Cl. CPC C12N 15/11; C12N 9/22; C12N 9/93; C12N 15/907; C12N 2310/20; C12N 2310/315

(57) ABSTRACT

The present disclosure provides a polynucleotide comprising an RNA guide sequence, a Cas-binding region, and a DNA template sequence. The disclosure also provides compositions comprising a Cas nuclease or a Cas nickase and one or more polynucleotides comprising a guide sequence, a Cas binding region, and a DNA template sequence. The disclosure further provides a fusion protein comprising a Cas nuclease or a Cas nickase and a DNA polymerase recruitment moiety. Also provided are methods for providing a targeted insertion in a target DNA of a cell.

### FIELD OF THE INVENTION

The present disclosure provides a polynucleotide comprising an RNA guide sequence, a Cas-binding region, and a DNA template sequence. The disclosure also provides compositions comprising a Cas nuclease or a Cas nickase and one or more polynucleotides comprising a guide sequence, a Cas binding region, and a DNA template sequence. The disclosure further provides a fusion protein comprising a Cas nuclease or a Cas nickase and a DNA polymerase recruitment moiety. Also provided are methods for providing a targeted insertion in a target DNA of a cell.

### BACKGROUND

Programmable nucleases such as CRISPR/Cas9 can generate site-specific double-stranded breaks (DSBs) that can disrupt genes by inducing mixtures of insertions and deletions (indels) at target sites. However, DSB repair relying on the template-dependent homology-directed repair (HDR) can have low frequency, while the high efficiency template-independent non-homologous end joining (NHEJ) can be error-prone and may not favor desired insertions.

Anzalone et al. (Nature 576: 149-157 (2019)) described the development of prime editing, which utilizes a Cas9 nickase-reverse transcriptase fusion enzyme to insert short sequences at the site of cleavage. Prime editing relies upon a complex mechanism of RNA removal and hybridization of single-stranded DNA to a target site, and also requires removal of an overlapping “flap” sequence by cellular equilibrium.

### SUMMARY OF THE INVENTION

In some embodiments, the disclosure provides a polynucleotide comprising (i) an RNA guide sequence; (ii) a Cas-binding region; and (iii) a DNA template sequence, wherein the DNA template sequence is at a 3′ end of the polynucleotide.

In some embodiments, the DNA template sequence comprises a modified nucleotide, a non-B DNA structure, a DNA polymerase recruitment moiety, a DNA ligase recruitment moiety, or a combination thereof.

In some embodiments, the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof. In some embodiments, the covalent linker comprises triethylene glycol (TEG). In some embodiments, the DNA lesion comprises 8oxoguanine, thymine-glycol, or a combination thereof. In some embodiments, the DNA photoproduct comprises a cyclobutane pyrimidine dimer (CPD), a pyrimidine (6-4) pyrimidone photoproduct, or a combination thereof. In some embodiments, the modified deoxyribonucleoside comprises deoxyuridine. In some embodiments, the methylated nucleotide comprises 5-hydroxymethylcytosine, 5-methylcytosine, or a combination thereof.

In some embodiments, the non-B DNA structure comprises a hairpin, a cruciform, Z-DNA, H-DNA (triplex DNA), G-quadruplex DNA (tetraplex DNA), slipped DNA, sticky DNA, or a combination thereof. In some embodiments, the DNA polymerase recruitment moiety comprises a DNA polymerase recruitment protein linked to the DNA template sequence. In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof. In some embodiments, the DNA ligase recruitment moiety comprises a 5′ adenylation of the DNA template sequence.

In some embodiments, the disclosure provides a polynucleotide comprising: (i) an RNA guide sequence; (ii) a Cas-binding region; and (iii) a DNA template sequence, wherein the DNA template sequence is at a 3′ end of the polynucleotide, and wherein the DNA template sequence comprises a phosphorothioate bond. In some embodiments the DAN template sequence is at a 5′ end of the polynucleotide

In some embodiments, the DNA template sequence comprises a primer binding sequence and a sequence of interest. In some embodiments, the Cas-binding region comprises RNA, or wherein the Cas-binding region comprises a combination of RNA and DNA. In some embodiments, the Cas-binding region comprises a tracrRNA. In some embodiments, the Cas-binding region is capable of hybridizing to a tracrRNA.

In some embodiments, the tracrRNA is capable of binding to a Cas nuclease. In some embodiments, the Cas nuclease is Cas9 or Cas12a. In some embodiments, the Cas nuclease is a Type II-B Cas. In some embodiments, the tracrRNA is capable of binding to a Cas nickase. In some embodiments, the Cas nickase is a Cas9 nickase, a Cas12a nickase, or a Type II-B Cas nickase.

In some embodiments, the DNA template sequence is about 8 to about 10000 nucleotides in length. In some embodiments, the primer binding sequence is about 4 to about 300 nucleotides in length. In some embodiments, the sequence of interest is about 4 to about 100 nucleotides in length. In some embodiments, the RNA guide sequence is about 15 to about 25 nucleotides in length.

In some embodiments, the polynucleotide further comprises a spacer positioned between the Cas-binding region and the DNA template sequence. In some embodiments, the spacer comprises a stop sequence for a DNA polymerase. In some embodiments, the spacer comprises more than one stop sequence. In some embodiments, the stop sequence comprises a secondary structure. In some embodiments, the secondary structure comprises a stem loop. In some embodiments, the spacer is about 10 to about 200 nucleotides in length.

In some embodiments, the disclosure provides a cell comprising the polynucleotide described herein.

In some embodiments, the disclosure provides a composition comprising: a Cas nuclease or a Cas nickase; and a polynucleotide comprising: (i) a guide sequence; (ii) a Cas-binding region; and (iii) a DNA template sequence, wherein the DNA template sequence is at a 3′ end of the polynucleotide.

In some embodiments, the disclosure provides a composition comprising: a Cas nuclease or a Cas nickase; a first polynucleotide comprising: (i) a guide sequence; (ii) a Cas-binding region; and (iii) a first hybridization region, wherein the first hybridization region is at a 3′ end of the first polynucleotide; and a second polynucleotide comprising: (i) a second hybridization region that is complementary to the first hybridization region and (ii) a DNA template sequence.

In some embodiments, the disclosure provides a composition comprising: a Cas nuclease or a Cas nickase; a first polynucleotide comprising: (i) a guide sequence; and (ii) a first hybridization region, wherein the first hybridization region is at a 3′ end of the first polynucleotide; and a second polynucleotide comprising: (i) a second hybridization region that is complementary to the first hybridization region; (ii) a Cas-binding region; and (iii) a DNA template sequence.

In some embodiments, the DNA template sequence comprises a primer binding sequence and a sequence of interest. In some embodiments, the DNA template sequence comprises a modified nucleotide, a non-B DNA structure, a DNA polymerase recruitment moiety, a DNA ligase recruitment moiety, or a combination thereof.

In some embodiments, the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof. In some embodiments, the modified nucleotide comprises a phosphorothioate bond. In some embodiments, the covalent linker comprises triethylene glycol (TEG). In some embodiments, the DNA lesion comprises 8-oxoguanine, thymine-glycol, or a combination thereof. In some embodiments, the DNA photoproduct comprises a cyclobutane pyrimidine dimer (CPD), a pyrimidine (6-4) pyrimidone photoproduct, or a combination thereof. In some embodiments, the deoxyribonucleoside comprises deoxyuridine. In some embodiments, the methylated nucleotide comprises 5-hydroxymethylcytosine, 5-methylcytosine, or a combination thereof.

In some embodiments, the non-B DNA structure comprises a hairpin, a cruciform, Z-DNA, H-DNA (triplex DNA), G-quadruplex DNA (tetraplex DNA), slipped DNA, sticky DNA, or a combination thereof. In some embodiments, the DNA polymerase recruitment moiety comprises a DNA polymerase recruitment protein linked to the DNA template sequence. In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof. In some embodiments, the DNA ligase recruitment moiety comprises a 5′ adenylation of the DNA template sequence.

In some embodiments, the guide sequence comprises RNA, or wherein the guide sequence comprises a combination of RNA and DNA. In some embodiments, the Cas-binding region comprises RNA, or wherein the Cas-binding region comprises a combination of RNA and DNA. In some embodiments, the Cas-binding region comprises a tracrRNA. In some embodiments, the Cas-binding region is capable of hybridizing to a tracrRNA, and wherein the composition further comprises a tracrRNA.

In some embodiments, the tracrRNA is capable of binding to the Cas nuclease or Cas nickase. In some embodiments, the composition comprises a Cas nuclease. In some embodiments, the Cas nuclease is Cas9 or Cas12a. In some embodiments, the composition comprises a Cas nuclease, and wherein the Cas nuclease is a Type II-B Cas. In some embodiments, the composition comprises a Cas nickase. In some embodiments, the Cas nickase is a Cas9 nickase, a Cas12a nickase, or a Type II-B Cas nickase.

In some embodiments, the DNA template sequence is about 8 to about 500 nucleotides in length. In some embodiments, the primer binding sequence is about 4 to about 30 nucleotides in length. In some embodiments, the sequence of interest is about 4 to about 100 nucleotides in length. In some embodiments, the RNA guide sequence is about 15 to about 25 nucleotides in length.

In some embodiments, the first hybridization region and the second hybridization region are RNA. In some embodiments, the first hybridization region and the second hybridization region are single-stranded DNA. In some embodiments, the first hybridization region is RNA and the second hybridization region is single-stranded DNA, or wherein the first hybridization region is single-stranded DNA and the second hybridization region is RNA. In some embodiments, the first hybridization region is about 4 to about 5000 nucleotides in length. In some embodiments, the second hybridization region is about 4 to about 5000 nucleotides in length.

In some embodiments, the polynucleotide further comprises a spacer positioned 5′ of the DNA template sequence. In some embodiments, the second polynucleotide further comprises a spacer position 5′ of the DNA template sequence. In some embodiments, the spacer comprises a stop sequence for a DNA polymerase. In some embodiments, the spacer comprises more than one stop sequence. In some embodiments, the stop sequence comprises a secondary structure. In some embodiments, the secondary structure comprises a stem loop. In some embodiments, the spacer is about 10 to about 200 nucleotides in length.

In some embodiments, the Cas nuclease or the Cas nickase is fused to a DNA polymerase recruitment protein. In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

In some embodiments, the disclosure provides a cell comprising the composition described herein. In some embodiments, the cell further comprises an exogenous DNA polymerase, an exogenous DNA ligase, or both.

In some embodiments, the disclosure provides a fusion protein comprising (i) a Cas nuclease or a Cas nickase; and (ii) a DNA polymerase recruitment protein.

In some embodiments, the fusion protein comprises a Cas nuclease. In some embodiments, the Cas nuclease is Cas9 or Cas12a. In some embodiments, the Cas nuclease is a Type II-B Cas. In some embodiments, the fusion protein comprises a Cas nickase. In some embodiments, the Cas nickase is a Cas9 nickase, a Cas12a nickase, or a Type II-B Cas nickase.

In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

In some embodiments, the disclosure provides a polynucleotide encoding the fusion protein described herein. In some embodiments, the disclosure provides a vector comprising the polynucleotide that encodes the fusion protein. In some embodiments, the disclosure provides a cell comprising the fusion protein, the vector, or the polynucleotide. In some embodiments, the cell further comprises a polynucleotide described herein that comprises (i) a RNA guide sequence, (ii) a Cas-binding region; and (iii) a DNA template sequence, wherein the DNA template sequence is at a 3′ end of the polynucleotide

In some embodiments, the disclosure provides a method of providing a targeted insertion in a target DNA in a cell, comprising introducing the composition described herein into the cell, wherein the guide sequence is capable of hybridizing to the target DNA.

In some embodiments, the method does not comprise introducing a DNA polymerase into the cell. In some embodiments, the Cas nuclease generates a double-stranded cleavage in the target DNA, and an endogenous DNA polymerase of the cell extends the DNA template sequence.

In some embodiments, the method further comprises introducing an exogenous DNA polymerase into the cell. In some embodiments, the Cas nuclease generates a double-stranded cleavage in the target DNA, and the exogenous DNA polymerase extends the DNA template sequence.

In some embodiments, the DNA template sequence comprises a primer binding sequence and a sequence of interest, and the DNA polymerase synthesizes a DNA strand complementary to the sequence of interest to form a double-stranded sequence comprising the sequence of interest. In some embodiments, the double-stranded sequence is inserted into the cleaved target DNA. In some embodiments, the double-stranded sequence is inserted into the cleaved target DNA by non-homologous end joining (NHEJ). In some embodiments, the double-stranded sequence is inserted into the cleaved target DNA by a DNA ligase. In some embodiments, the DNA ligase is an endogenous DNA ligase of the cell. In some embodiments, the DNA ligase is an exogenous DNA ligase.

In some embodiments, the disclosure is directed to a composition comprising: (a) a Cas nuclease or a Cas nickase; (b) a first polynucleotide comprising: (i) a guide sequence; (ii) a Cas-binding region; (iii) a first hybridization region; and (iv) a primer-binding sequence, wherein the primer-binding sequence is at a 3′ end of the first polynucleotide; and (c) a second polynucleotide comprising: (i) a second hybridization region that is complementary to the first hybridization region; and (ii) a sequence of interest (SOI).

In some embodiments, the disclosure is directed to a composition comprising: (a) a Cas nuclease or a Cas nickase; (b) a first polynucleotide comprising: (i) a guide sequence; (ii) a Cas-binding region; and (iii) a first hybridization region, wherein the first hybridization region is at a 3′ end of the first polynucleotide; (c) a second polynucleotide comprising: (i) a second hybridization region that is complementary to the first hybridization region; (ii) a third hybridization region; and (iii) a primer-binding sequence, wherein the primer-binding sequence is at a 3′ end of the second polynucleotide; and (d) a third polynucleotide comprising: (i) a fourth hybridization region that is complementary to the third hybridization region; and (ii) a sequence of interest (SOI).

In some embodiments, the disclosure is directed to a fusion protein comprising (i) a Cas nuclease or a Cas nickase; and (ii) a DNA polymerase recruitment protein, a DNA ligase, a DNA ligase recruitment moiety, a DNA binding protein, a DNA repair protein, or combination thereof.

In some embodiments, the disclosure provides a method of providing a targeted insertion in a target DNA in a cell, comprising introducing a composition as described herein into the cell, wherein the guide sequence is capable of hybridizing to the target DNA.

### BRIEF DESCRIPTION OF THE DRAWINGS

The following drawings form part of the present specification and are included to further demonstrate exemplary embodiments of certain aspects of the present invention.

FIG. 1 illustrates an exemplary method described in embodiments herein. A Cas nuclease is guided to a target DNA in a cell by a guide sequence and cleaves the target DNA. The DNA template sequence comprises (i) a primer binding sequence that hybridizes with the cleaved DNA, which serves as a primer, and (ii) a sequence of interest. A DNA polymerase, e.g., endogenous DNA polymerase of the cell, binds to the primer and synthesizes a DNA strand complementary to the sequence of interest, thereby forming a double-stranded sequence comprising the sequence of interest. The double-stranded sequence can be inserted into the cleaved target DNA by a DNA repair pathway, e.g., NHEJ.

FIGS. 2A-2D illustrate exemplary polynucleotide constructs (“springRNA”) tested in an in vivo targeted insertion experiment described herein. FIG. 2A shows an RNA-only polynucleotide comprising an RNA guide sequence, tracrRNA, and RNA template sequence. FIG. 2B shows an RNA-DNA hybrid polynucleotide comprising an RNA guide sequence, tracrRNA, and DNA template sequence (“DNA”) or DNA template sequence with phosphorothioate bonds (“PS-DNA”). FIG. 2C shows an RNA-only polynucleotide comprising an RNA guide sequence, tracrRNA, and RNA template sequence with an abasic site. FIG. 2D shows an RNA-only polynucleotide comprising an RNA guide sequence, tracrRNA, and RNA template sequence with a TEG.

FIG. 3 relates to Example 1 and shows the results of targeted insertions using SpCas9 only or a SpCas9-reverse transcriptase (SpCas9-RT) fusion protein with various springRNA constructs. Panels A-E of FIG. 3 show targeted insertions with SpCas9 and each of the springRNA constructs in FIGS. 2A-2D. Panels F-J of FIG. 3 show targeted insertions with SpCas9-RT (“PE0”) and each of the springRNA constructs in FIGS. 2A-2D.

FIG. 4 relates to Example 1 and shows the relative frequency of insertions by SpCas9 or SpCas9-RT in combination with each of the springRNA constructs in FIGS. 2A-2D. Each point represents an independent replicate. Mean and standard deviations of three experiments are indicated as a whisker plot.

FIGS. 5A-5F relate to Example 2 and show the results of targeted insertions using SpCas9 only or SpCas9-RT. FIGS. 5A-5C show the targeted insertions with SpCas9-RT (“PE0”) and RNA-only springRNA (FIG. 5A), springRNA with DNA tail (FIG. 5B), and springRNA with PS-DNA (FIG. 5C). FIGS. 5D-5F show the targeted insertions with SpCas9 and RNA-only springRNA (FIG. 5D), springRNA with DNA tail (FIG. 5E), and springRNA with PS-DNA (FIG. 5F).

FIG. 6 shows an overview of an in vitro assay described herein. Two complementary DNA strands are labeled with different fluorophores (6 FAM-labeled non-target strand and HEX-labeled target strand). A guide sequence is designed such that Cas9 cleavage generates two strands of different lengths. A DNA polymerase extends the 6 FAM-labeled non-target strand hybridized to the primer binding sequence of the springRNA. The products are denatured and separated by capillary electrophoresis, and the fluorophore-coupled strands are detected.

FIGS. 7A-7F relate to Example 3 and show the results of the in vitro assay illustrated in FIG. 6. The blue traces correspond to the non-target strand, and green traces correspond to the target strand. The asterisks indicate the cleaved synthetic target DNA substrate, and black arrows indicate extension products by the DNA polymerase. FIG. 7A shows the assay results with Cas9 and springRNA. FIG. 7B shows the assay results with Cas9, Klenow fragment, and springRNA. FIGS. 7C-7F show the assay results with Cas9, Bst 3.0 polymerase, and: springRNA (FIG. 7C and FIG. 7D), springRNA with abasic site (FIG. 7E), or springRNA with TEG (FIG. 7F).

FIG. 8 shows the extension reaction kinetics of Bst 3.0 polymerase and Klenow fragment, the DNA polymerases used in the in vitro assay of Example 3.

FIG. 9 illustrates an exemplary composition described in embodiments herein, comprising a Cas protein (e.g., Cas nuclease or Cas nickase); a first polynucleotide that comprises the guide sequence (referred to as “spacer”), Cas-binding region (referred to as “gRNA scaffold”), first hybridization region (referred to as “landing pad”), and primer binding sequence (PBS); and a second polynucleotide comprises the second hybridization region (referred to as “hybridization sequence”), SOI (referred to as “insert”), and homology sequence. The terminology for various components of the composition in FIG. 9 are used throughout FIGS. 9-23.

FIGS. 10A-10C illustrate an exemplary method described in embodiments herein. In FIG. 10A, the guide sequence, Cas-binding region, first hybridization region, and primer binding site are located on a first polynucleotide. A double-stranded break is generated at a target DNA by a Cas nuclease guided to the target DNA by a guide sequence. The primer binding site is hybridized to the cleaved DNA, and a second polynucleotide hybridizes to the first polynucleotide via the first and second hybridization regions that comprise complementary sequences. The second polynucleotide comprises a sequence of interest, which is ligated into the cleaved DNA at both the 5′ and 3′ ends by a ligase (FIG. 10A). The second polynucleotide can further include a homology sequence, and the 5′ end is ligated into the cleaved DNA by a ligase, and the 3′ end is integrated by a homology-mediated mechanism (FIG. 10B). Following the ligation and/or homology-mediated integration, the complex is converted into double-stranded DNA, thereby integrating the sequence of interest into the target DNA (FIG. 10C).

FIGS. 11(A)-11(J) illustrate exemplary embodiments of the compositions described herein. FIG. 11(A) depicts the donor containing all three elements: second hybridization sequence, the sequence of interest, and the homology sequence. FIG. 11(B) depicts donor without homology sequence. FIG. 11(C) depicts donor without the second hybridization sequence. FIG. 11(D) represents a standard donor, with a sequence complementary to the sequence of interest hybridized to form a double stranded region sequence of interest with both 5′ and 3′ overhangs. This configuration is termed “overhang.” FIG. 11(E) depicts a donor as in FIG. 11(A), but the plugRNA is composed of two nucleic acids. The sgRNA has a ˜30 nt long sequence added to the 3′ end. This 3′ sequence can pair with a complimentary polynucleotide, which is then followed by the first hybridization sequence and the primer binding sequence. This configuration is termed “split plugRNA” or “split system.” FIG. 11(F) depicts a configuration as in FIG. 11(A), but the hybridization sequence is shorter, generating a gap between the 3′ end of the primed target site and 5′ end of the donor. This configuration is termed “gap.” FIG. 11(G) is the same as in FIG. 11(A), but the donor has additional nucleotides at the 5′ end, generating a flap, that in some embodiments may engage FEN1 and other repair machinery. This configuration is termed “flap.” FIG. 11(H) is the same as in FIG. 11(B), but a sequence complementary to the sequence of interest is annealed, generating a blunt end on the 3′ end of the donor. This configuration is termed “blunt.” FIG. 11(I) the donor is split into two polynucleotides. The first donor strand comprises second hybridization sequence and a sequence of interest. The second sequence comprises the homology sequence and a sequence complementary to the sequence of interest. This configuration is termed “bridge.” In FIG. 11(J), the homology sequence is embedded in the plugRNA, immediately downstream of the gRNA scaffold. This homology sequence anneals to the 3′ side of the Cas-induced break. The donor comprises the sequence of interest flanked by two hybridization sequences: the second hybridization sequence and a further hybridization sequence, the second hybridization sequence complementary to the first hybridization sequence, and the further hybridization complementary to a region adjacent to the homology sequence. This configuration brings both 5′ and 3′ sides of the donor in proximity to the double strand break. This configuration is termed “dual hybridization.”

FIG. 12A shows the experimental design of an exemplary in vitro assay as described in Example 8. FIGS. 12B and 12C show the results of the assay, with FIG. 12B showing the percent ligation product and FIG. 12C showing the percent ligation efficiency.

FIG. 13A shows the experimental design of an exemplary in vitro ligation assay using HeLa nuclear extracts, as described in Example 9. FIGS. 13B and 13C show heat map results of the assay, with FIG. 13B showing percent ligation product and FIG. 13C showing percent ligation efficiency.

FIG. 14A shows the experimental design of an exemplary assay using HeLa nuclear extracts, wherein the resulting DNA were amplified and analyzed for targeted insertions, as described in Example 10. FIG. 14B shows representation next-generation sequencing (NGS) results of the targeted insertion sequences.

FIG. 15 shows the results of an exemplary assay performed with compositions of the various configurations shown in FIG. 11A-11J, as described in Example 11.

FIG. 16A shows an exemplary embodiment of a composition described herein. FIG. 16B shows the results of exemplary assays performed with varying lengths of the different components of the composition, as described in Example 12.

FIG. 17A shows exemplary embodiments of compositions described herein. FIG. 17B shows the results of exemplary assays performed with varying lengths of the different components of the composition, as described in Example 13.

FIG. 18A shows exemplary embodiments of compositions described herein. FIG. 18B shows the results of exemplary assays performed with varying lengths and/or modifications of the different components of the composition, as described in Example 14.

FIG. 19A shows an exemplary embodiment of a composition described herein. FIG. 19B shows the results of exemplary assays performed with varying lengths of the different components of the composition, as described in Example 15.

FIG. 20A shows exemplary embodiments of compositions described herein. FIG. 20B shows the results of exemplary assays performed with varying lengths and/or modifications of the different components of the composition and in the presence or absence of a DNA-dependent protein kinase (DNA-PK) inhibitor, as described in Example 16.

FIG. 21A shows exemplary embodiments of compositions described herein. FIG. 21B shows the results of exemplary assays performed with the various composition configurations and in the presence of a co-expressed DNA ligase, as described in Example 17.

FIG. 22 shows a summary of the results of assays performed with the different configurations of the compositions described herein, as described in Example 18.

FIG. 23A shows an exemplary embodiment of a composition described herein. FIG. 23B shows the results of an exemplary assay performed with the composition and different ligases or DNA polymerase recruitment proteins, as described in Example 19.

### DETAILED DESCRIPTION OF THE INVENTION

The present disclosure relates to improved CRISPR systems and components thereof, and methods of using the same. In general, a CRISPR system, e.g., a CRISPR/Cas system, includes elements that promote the formation of a CRISPR complex, such as a guide polynucleotide and a Cas protein, at the site of a target polynucleotide, e.g., a target DNA sequence. In naturally-occurring CRISPR systems (e.g., the bacterial immunity CRISPR/Cas9 system), foreign DNA is incorporated into CRISPR arrays, which then produce CRISPR-RNAs (crRNA). The crRNA includes RNA guide sequence regions complementary to the foreign DNA site and hybridizes with trans-activating CRISPR-RNA (tracrRNA), which is also encoded by the CRISPR system. The tracrRNA forms secondary structures, e.g., stem loops, and is capable of binding to Cas9 protein. The crRNA/tracrRNA hybrid associates with Cas9, and the crRNA/tracrRNA/Cas9 complex recognizes and cleaves foreign DNA bearing the protospacer sequences, thereby conferring immunity against the invading virus or plasmid. CRISPR/Cas systems are further described in, e.g., Jinek et al., Science 337(6096):816-821 (2012); Cong et al., Science 339(6121):819-823 (2013); Mali et al., Science 339(6121):823-826 (2013); and Sander et al., Nat Biotechnol 32:347-355 (2014).

CRISPR/Cas systems have been engineered to introduce insertions into a target polynucleotide, also known as targeted insertions. Typically, the guide polynucleotide is designed such that the Cas protein generates a double-stranded cleavage at the target polynucleotide, and a separate donor template comprising the sequence of interest is inserted into the cleaved target polynucleotide by cellular DNA repair mechanisms, e.g., non-homologous end joining (NHEJ) or homology-directed repair (HDR). The efficiency of insertion is dependent on several factors, including transfection ratio of the donor template, Cas protein, and guide polynucleotide; sequence and size of the donor template; and type of DNA repair mechanism triggered. For example, HDR provides high-fidelity DNA repair but has low insertion frequency, while NHEJ has higher insertion frequency but may also introduce mutations into the target DNA.

In some embodiments, the present disclosure provides compositions, polynucleotides, and/or fusion proteins for improved targeted insertion methods. In some embodiments, the compositions, polynucleotides, and/or fusion proteins of the present disclosure provide higher precision of inserting a sequence of interest. In some embodiments, the compositions, polynucleotides, and fusion proteins of the present disclosure provide higher efficiency of inserting a sequence of interest.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7% 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any protein, compositions, polynucleotides, vectors, cells, methods, and/or kits of the present disclosure. Furthermore, compositions, polynucleotides, vectors, cells, and/or kits of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

A “nucleic acid,” “nucleic acid molecule,” “nucleotide,” “nucleotide sequence,” “oligonucleotide,” or “polynucleotide” means a polymeric compound including covalently linked nucleotides. The term “nucleic acid” includes ribonucleic acid (RNA) or deoxyribonucleic acid (DNA) both of which may be single- or double-stranded. The polynucleotide may comprise naturally-occurring nucleobases (e.g., guanine, adenine, cytosine, thymine, and uracil), modified nucleobases (e.g., hypoxanthine, xanthine, 7-methylguanine, dihydrouracil, 5-methylcytosine, 5-hydroxymethylcytosine), and/or artificial nucleobases (e.g., isoguanine or isocytosine). Nucleic acids are transcribed from a 5′ end to a 3′ end. In some embodiments, the disclosure provides a polynucleotide comprising RNA and DNA nucleotides. Methods of producing a polynucleotide comprising both RNA and DNA nucleotides are known in the art and include, e.g., ligation or oligonucleotide synthesis methods. In some embodiments, the disclosure provides a polynucleotide capable of forming a complex with a Cas nuclease or Cas nickase as described herein. In some embodiments, the disclosure provides a polynucleotide encoding any one of the proteins disclosed herein, e.g., a Cas nuclease or Cas nickase.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the stringency of the hybridization. The stringency of the hybridization conditions can be selected to provide selective formation or maintenance of a desired hybridization product of two complementary polynucleotides, in the presence of other potentially cross-reacting or interfering polynucleotides. Stringent conditions are sequence-dependent; typically, longer complementary sequences specifically hybridize at higher temperatures than shorter complementary sequences. Generally, stringent hybridization conditions are between about 5° C. to about 10° C. lower than the thermal melting point Tₘ (i.e., the temperature at which 50% of the sequences hybridize to a substantially complementary sequence) for a specific polynucleotide at a defined ionic strength, concentration of chemical denaturants, pH, and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, and/or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations or ionic strength of less than about 1 M, 500 mM, 200 mM, 100 mM or 50 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

The term “complementary” is used to describe the relationship between nucleotide bases that are capable of hybridizing to one another. For example, with respect to DNA, adenosine is complementary to thymine and cytosine is complementary to guanine. When two nucleic acids are “complementary,” it is meant that a first nucleic acid or one or more regions thereof is capable of hydrogen bonding with a second nucleic acid or one or more regions thereof. Complementary nucleic acids need not have complementarity at each nucleotide and may include one or more nucleotide mismatches, i.e., points at which hydrogen bonding does not occur. For example, complementary oligonucleotides can have at least 80%, at least 85%, at least 90%, at least 95%, at least 96%, at least 97%, at least 98%, or at least 99% of nucleotides hydrogen bond. By contrast, “fully complementary” or “100% complementary” in reference to oligonucleotides means that each nucleotide hydrogen bonds without any nucleotide mismatches.

The term “homologous recombination” refers to the insertion of a foreign polynucleotide (e.g., DNA) into another nucleic acid (e.g., DNA) molecule, e.g., insertion of a vector in a chromosome. In some cases, the vector targets a specific chromosomal site for homologous recombination. For specific homologous recombination, the vector typically contains sufficiently long regions of homology to sequences of the chromosome to allow complementary binding and incorporation of the vector into the chromosome. Longer regions of homology and greater degrees of sequence similarity may increase the efficiency of homologous recombination. In some embodiments, the polynucleotides or compositions described herein facilitate homologous recombination by generating breaks, e.g., double-stranded breaks in a nucleic acid sequence.

As used herein, the term “operably linked” means that a polynucleotide of interest, e.g., the polynucleotide encoding a nuclease, is linked to the regulatory element in a manner that allows for expression of the polynucleotide. Regulatory elements can be cis-regulatory elements or trans-regulatory elements. Regulatory elements include, for example, promoters, enhancers, terminators, 5′ and 3′ UTRs, insulators, silencers, operators, and the like. In some embodiments, the regulatory element is a promoter. In some embodiments, a polynucleotide expressing a protein of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments, the vector is an episomal vector, which is removed/lost from a population of cells after a number of cellular generations, e.g., by asymmetric partitioning. The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. A vector may include one or more regulatory regions, and/or selectable markers useful in selecting, measuring, and monitoring nucleic acid transfer results (transfer to which tissues, duration of expression, etc.).

Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating polynucleotides (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, adenovirus, adeno-associated virus, pox, baculovirus, vaccinia, herpes simplex, Epstein-Barr, adenovirus, geminivirus, and caulimovirus vectors. In some embodiments, a viral vector is utilized to provide the polynucleotides described herein. In some embodiments, a viral vector is utilized to provide a polynucleotide coding for a protein described herein.

Vectors may be introduced into the desired host cells by known methods, including, but not limited to, transfection, transduction, cell fusion, and lipofection. Vectors can include various regulatory elements including promoters. In some embodiments, vector designs can be based on constructs designed by Mali et al., Nat Methods 10: 957-63 (2013).

Methods known in the art may be used to propagate polynucleotides and/or vectors provided herein. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of polynucleotides have been joined or recombined into a unique construction which is capable of introducing a promoter fragment and DNA sequence for a selected gene product along with appropriate 3′ untranslated sequence into a cell. In some embodiments, a plasmid is utilized to provide the polynucleotides described herein. In some embodiments, a plasmid is utilized to provide a polynucleotide coding for a protein described herein.

The term “transfection” as used herein means the introduction of an exogenous nucleic acid molecule, including a vector, into a cell. Transfection methods, e.g., for components of the CRISPR/Cas compositions described herein, are known to one of ordinary skill in the art. A “transfected” cell includes an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to herein as “recombinant,” “transformed,” or “transgenic” organisms. In some embodiments, the present disclosure provides a host cell comprising any of the expression vectors described herein, e.g., an expression vector comprising a polynucleotide that encodes a protein described herein.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.”

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photocross-linking moieties, uniquely reactive functional groups, and NMR, IR, and x-ray crystallographic probes. Exemplary unnatural or synthetic amino acids are provided in, e.g., Mitra et al., Mater Methods 3:204 (2013) and Wals et al., Front Chem 2:15 (2014). Unnatural amino acids may also include naturally-occurring compounds that are not typically incorporated into a protein or polypeptide, such as, e.g., citrulline (Cit), selenocysteine (Sec), and pyrrolysine (Pyl).

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “exogenous” means that the referenced molecule or activity introduced into the host cell. The molecule can be introduced, for example, by introduction of an encoding nucleic acid into the host genetic material, such as by integration into a host chromosome or as non-chromosomal genetic material, e.g., a plasmid. An “exogenous” protein can be introduced into a host cell via an “exogenous” nucleic acid encoding the protein. The term “endogenous” refers to a referenced molecule or activity that is naturally present in the host cell. An “endogenous” protein is expressed by a nucleic acid contained within the host cell. The term “heterologous” refers to a molecule or activity derived from a source other than the referenced organism/species, whereas “homologous” refers to a molecule or activity derived from the host organism/species. Accordingly, exogenous expression of an encoding nucleic acid can utilize either or both of a heterologous or homologous encoding nucleic acid.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

The term “motif,” when used in reference to a polypeptide or protein, generally refers to a set of conserved amino acid residues, typically shorter than 20 amino acids in length, that may be important for protein function. Specific sequence motifs may mediate a common function, such as protein-binding or targeting to a particular subcellular location, in a variety of proteins. Examples of motifs include, but are not limited to, nuclear localization signals, microbody targeting motifs, motifs that prevent or facilitate secretion, and motifs that facilitate protein recognition and binding. Motif databases and/or motif searching tools are known in the field and include, for example, PROSITE, PFAM, PRINTS, and MiniMotif Miner.

An “engineered” protein, as used herein, means a protein that includes one or more modifications in a protein to achieve a desired property. Exemplary modifications include, but are not limited to, insertion, deletion, substitution, and/or fusion with another domain or protein. A “fusion protein” (also termed “chimeric protein”) is a protein comprising at least two domains, typically coded by two separate genes, that have been joined such that they are transcribed and translated as a single unit, thereby producing a single polypeptide having the functional properties of each of the domains. Engineered proteins of the present disclosure include Cas nucleases, Cas nickases, and fusions of Cas proteins with a DNA polymerase, DNA ligase, and/or DNA polymerase-binding protein.

In some embodiments, engineered protein is generated from a wild-type protein. As used herein, a “wild-type” protein or nucleic acid is a naturally-occurring, unmodified protein or nucleic acid. For example, a wild-type Cas9 protein can be isolated from the organism Streptococcus pyogenes. Wild-type can be contrasted with “mutant,” which includes one or more modifications in the amino acid and/or nucleotide sequence of the protein or nucleic acid. In some embodiments, an engineered protein can have substantially the same activity as a wild-type protein, e.g., greater than about 80%, greater than about 85%, greater than about 90%, greater than about 95%, or greater than about 99% of the activity as a wild-type protein. In some embodiments, the Cas nuclease of a fusion protein described herein has substantially the same activity as a wild-type Cas nuclease.

As used herein, the terms “sequence similarity” or “% similarity” refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Moreover, the skilled artisan recognizes that similar polynucleotides encompassed by the present disclosure are also defined by their ability to hybridize, under stringent conditions, with the sequences exemplified herein. Similar polynucleotides of the present disclosure are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the polynucleotides disclosed herein.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: (i) positively-charged side chains: Arg, His, Lys; (ii) negatively-charged side chains: Asp, Glu; (iii) polar, uncharged side chains: Ser, Thr, Asn, Gln; (iv) hydrophobic side chains: Ala, Val, Ile, Leu, Met, Phe, Tyr, Trp; and (v) others: Cys, Gly, Pro.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso).

Percent identity of polynucleotides or polypeptides can be determined when the polynucleotide or polypeptide sequences are aligned over a specified comparison window. In some embodiments, only specific portions of two or more sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two amino acid sequences is determined using the algorithm of Karlin and Altschul, Proc Nat Acad Sci USA 87:2264-2268 (1990), modified as in Karlin and Altschul, Proc NatAcad Sci USA 90:5873-5877 (1993). Such algorithms are incorporated into BLAST programs, e.g., BLAST+ or the NBLAST and XBLAST programs described in Altschul et al., J Mol Biol, 215: 403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res 25(17): 3389-3402 (1997). When utilizing BLAST and Gapped BLAST programs, the default parameters of the respective programs (e.g., XBLAST and NBLAST) can be used.

In some embodiments, a polypeptide or polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or polynucleotide (or a fragment of the reference polypeptide or polynucleotide) provided herein. In some embodiments, a polypeptide or polynucleotide have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or polynucleotide (or a fragment of the reference polypeptide or nucleic acid molecule) provided herein.

As used herein, a “complex” refers to a group of two or more associated polynucleotides and/or polypeptides. In the context of complex formation, the terms “associate” or “association” refers to molecules bound to one another through electrostatic, hydrophobic/hydrophilic, and/or hydrogen bonding interaction, without being covalently attached. A molecule that comprises different moieties covalently attached to one another is known. In some embodiments, a complex is formed when all the components of the complex are present together, i.e., a self-assembling complex. In some embodiments, a complex is formed through chemical interactions between different components of the complex such as, for example, hydrogen-bonding. In some embodiments, the polynucleotides provided herein form a complex with the proteins provided herein through secondary structure recognition of the polynucleotide by the protein. In some embodiments, the Cas-binding region of the polynucleotides provided herein comprise a secondary structure recognized by a Cas nuclease, Cas nickase, or fusion protein provided herein.

### Compositions

In some embodiments, the disclosure provides a composition comprising: a Cas nuclease or a Cas nickase; and a polynucleotide comprising (i) a guide sequence; a Cas-binding region; and (iii) a DNA template sequence, wherein the DNA template sequence is at a 3′ end of the polynucleotide.

In some embodiments, the disclosure provides a composition comprising: a Cas nuclease or a Cas nickase; a first polynucleotide comprising: (i) a guide sequence; (ii) a Cas-binding region; and (iii) a first hybridization region, wherein the first hybridization region is at a 3′ end of the first polynucleotide; and a second polynucleotide comprising: (i) a second hybridization region that is complementary to the first hybridization region and (ii) a DNA template sequence.

In some embodiments, the disclosure provides a composition comprising: a Cas nuclease or a Cas nickase; a first polynucleotide comprising: (i) a guide sequence; and (ii) a first hybridization region, wherein the first hybridization region is at a 3′ end of the first polynucleotide; and a second polynucleotide comprising: (i) a second hybridization region that is complementary to the first hybridization region; (ii) a Cas-binding region; and (iii) a DNA template sequence.

In some embodiments, the disclosure provides a composition comprising: a Cas nuclease or a Cas nickase; a first polynucleotide comprising: (i) a guide sequence; (ii) a Cas-binding region; (iii) a first hybridization region; and (iv) a primer binding sequence, wherein the primer binding sequence is at a 3′ end of the first polynucleotide; and a second polynucleotide comprising: (i) a second hybridization region that is complementary to the first hybridization region; (ii) a sequence of interest (SOI).

In some embodiments, the disclosure provides a composition comprising: a Cas nuclease or a Cas nickase; a first polynucleotide comprising: (i) a guide sequence; (ii) a Cas-binding region; and (iii) a first hybridization region, wherein the first hybridization region is at a 3′ end of the first polynucleotide; a second polynucleotide comprising: (i) a second hybridization region that is complementary to the first hybridization region; (ii) a third hybridization region; and (iii) a primer binding sequence, wherein the primer binding sequence is at a 3′ end of the second polynucleotide; and a third polynucleotide comprising: (i) a fourth hybridization region that is complementary to the third hybridization region; (ii) a sequence of interest (SOI).

As used herein, a “Cas protein” encompasses both Cas nucleases and Cas nickases. Cas proteins are part of the CRISPR/Cas system described herein. CRISPR/Cas systems, which include a Cas protein and a polynucleotide (also referred to as a “guide polynucleotide”), can be utilized for site-specific genome modifications. In some embodiments, the CRISPR/Cas system comprises a Cas protein and a guide polynucleotide comprising a Cas-binding region (which binds and/or activates the Cas protein) and a guide sequence (which hybridizes to a target sequence), wherein the Cas protein and the guide polynucleotide form a complex as described herein. In some embodiments, the CRISPR/Cas system comprises a Cas protein, a first polynucleotide comprising a guide sequence, and a second polynucleotide comprising a Cas-binding region, wherein the first and second polynucleotides hybridize to each other and form a complex with the Cas protein.

CRISPR/Cas systems can be classified as Types I to VI based on the Cas protein in the system. For example, Cas9 is found in Type II systems, and Cas12 is found in Type V systems. Each Type can be further divided into subtypes. For example, Type II can include subtypes II-A, II-B, and II-C, and Type V can include subtypes V-A and V-B. Classification of CRISPR/Cas systems and Cas nucleases is further discussed in, e.g., Makarova et al., Methods Mol Biol 1311:47-75 (2015); Makarova et al., The CRISPR Journal October 2018; 325-336; and Koonin et al., Phil Trans R Soc B 374:20180087 (2018). Cas nucleases described herein can encompass any Type or variant, unless otherwise specified.

In some embodiments, the composition comprises a Cas nuclease. In general, a Cas nuclease is capable of generating a double-stranded polynucleotide cleavage, e.g., a double-stranded DNA cleavage. In general, a Cas nuclease can include one or more nuclease domains, such as RuvC and HNH, and can cleave double-stranded DNA. In some embodiments, a Cas nuclease comprises a RuvC domain and an HNH domain, each of which cleaves one strand of double-stranded DNA. In some embodiments, the Cas nuclease generates blunt ends. In some embodiments, the RuvC and HNH of a Cas nuclease cleaves each DNA strand at the same position, thereby generating blunt ends. In some embodiments, the Cas nuclease generates cohesive ends. In some embodiments, the RuvC and HNH of a Cas nuclease cleaves each DNA strand at different positions (i.e., cut at an “offset”), thereby generating cohesive ends. As used herein, the terms “cohesive ends,” “staggered ends,” or “sticky ends” refer to a nucleic acid fragment with strands of unequal length. In contrast to “blunt ends,” cohesive ends are produced by a staggered cut on a double-stranded nucleic acid (e.g., DNA). A sticky or cohesive end has protruding singles strands with unpaired nucleotides, or “overhangs,” e.g., a 3′ or a 5′ overhang.

In some embodiments, the Cas nuclease is a Cas9 nuclease. Exemplary Cas9 nucleases include, but are not limited to, the Cas9 from Streptococcus pyogenes, Streptococcus thermophilus, Streptococcus mutans, Listeria innocua, Neisseria meningitidis, Staphylococcus aureus, Klebisella pneumoniae, and numerous other bacteria. Further exemplary Cas9 nucleases are described in, e.g., U.S. Pat. Nos. 8,771,945; 9,023,649; 10,000,772; and 10,407,697. In some embodiments, the Cas9 nuclease is from S. pyogenes (SpCas9).

In some embodiments, the Cas nuclease is a Cas12 nuclease. In some embodiments, the Cas nuclease is a Cas12a nuclease (formerly known as “Cpf1” or “C2c1”). Cas12 nucleases are generally smaller than Cas9 nucleases and can typically generate cohesive ends. Exemplary Cas12 proteins include, but are not limited to, the Cas12 protein from Francisella novicida, Acidaminococcus sp., Lachnospiraceae sp., Prevotella sp., and numerous other bacteria. Further Cas12 nuclease are described in, e.g., U.S. Pat. No. 9,580,701; US 2016/0208243; Zetsche et al., Cell 163(3):759-771 (2015); and Chen et al., Science 360:436-439 (2018).

In some embodiments, the Cas nuclease is a Type II-B Cas nuclease. Type II-B Cas nucleases are capable of generating cohesive ends as described herein. Exemplary Type IIB Cas9 proteins include, but are not limited to, the Cas9 protein from Legionella pneumophila, Francisella novicida, Parasutterella excrementihominis, Sutterella wadsworthensis, Wolinella succinogenes, the sequenced gut metagenome MH0245_GL0161830.1, and numerous other bacteria. Further Type II-B Cas9 proteins are described in, e.g., WO 2019/099943. In some embodiments, the Type II-B Cas nuclease is from the sequenced gut metagenome MH0245_GL0161830.1 (MHCas9).

In some embodiments, the composition comprises a Cas nickase. A nickase, which generates a single-stranded cleavage on a double-stranded polynucleotide (e.g., DNA), is distinguished from a nuclease, which cleaves both strands of a double-stranded polynucleotide (e.g., DNA). As discussed herein, a wild-type Cas nuclease typically comprises two catalytic nuclease domains, RuvC and HNH, and each nuclease domain is responsible for cleavage of one strand of double-stranded DNA. Thus, in some embodiments, a Cas nickase comprises an amino acid mutation in a catalytic domain relative to a Cas nuclease. Cas nickases are further described in, e.g., Cho et al., Genome Res 24:132-141 (2013); Ran et al., Cell 154:1380-1389 (2013); and Mali et al., Nat Biotechnol 31:833-838 (2013).

In some embodiments, the Cas nickase is a Cas9 nickase. In some embodiments, the Cas nickase is a Cas12a nickase. In some embodiments, the Cas nickase is a Type II-B Cas nickase. In some embodiments, the Cas nickase is produced by providing a mutation in a Cas nuclease. For example, the SpCas9 nickase comprises a D10A mutation or H840A mutation relative to wild-type SpCas9 nuclease. It will be understood by one of ordinary skill in the art that alignment methods such as those described herein can be used to determine the corresponding amino acid residues in other Cas nucleases (e.g., Cas12a or Type II-B Cas nucleases) to produce a Cas nickase.

In some embodiments, the Cas nuclease or Cas nickase of the composition is not fused to a heterologous protein domain. In some embodiments, the Cas nuclease or Cas nickase is not fused to a DNA polymerase, a DNA ligase, or a reverse transcriptase.

In some embodiments, the Cas nuclease or Cas nickase of the composition is fused to a heterologous protein domain. In some embodiments, the Cas nuclease or Cas nickase is fused to a DNA polymerase, a DNA polymerase recruitment moiety, a DNA ligase, a DNA ligase recruitment moiety, a DNA binding protein, a DNA repair protein, or combination thereof. Fusion proteins comprising Cas nuclease or Cas nickase are further described herein.

### Polynucleotides

In some embodiments, the composition of the present disclosure comprises a polynucleotide. In some embodiments, the polynucleotide comprises: (i) a guide sequence; (ii) a Cas-binding region; and (iii) a DNA template sequence, wherein the DNA template sequence is at a 3′ end of the polynucleotide. In some embodiments, the guide sequence is an RNA guide sequence. In some embodiments, the polynucleotide comprises, in 5′ to 3′ order: the guide sequence (e.g., RNA guide sequence), the Cas-binding region, and the DNA template sequence.

In some embodiments, the composition of the present disclosure comprises first and second polynucleotides. In some embodiments, the first polynucleotide comprises: (i) a guide sequence; (ii) a Cas-binding region; and (iii) a first hybridization region, wherein the first hybridization region is at a 3′ end of the first polynucleotide; and the second polynucleotide comprises: (i) a second hybridization region that is complementary to the first hybridization region and (ii) a DNA template sequence. In some embodiments, the first polynucleotide comprises, in 5′ to 3′ order: the guide sequence, the Cas-binding region, and the first hybridization region. In some embodiments, the second polynucleotide comprises, in 5′ to 3′ order: the second hybridization region and the DNA template sequence.

In some embodiments, the first polynucleotide comprises: (i) a guide sequence; and (ii) a first hybridization region, wherein the first hybridization region is at a 3′ end of the first polynucleotide; and the second polynucleotide comprises: (i) a second hybridization region that is complementary to the first hybridization region; (ii) a Cas-binding region; and (iii) a DNA template sequence. In some embodiments, the first polynucleotide comprises, in 5′ to 3′ order: the guide sequence and the first hybridization region. In some embodiments, the second polynucleotide comprises, in 5′ to 3′ order: the second hybridization region, the Cas-binding region, and the DNA template sequence.

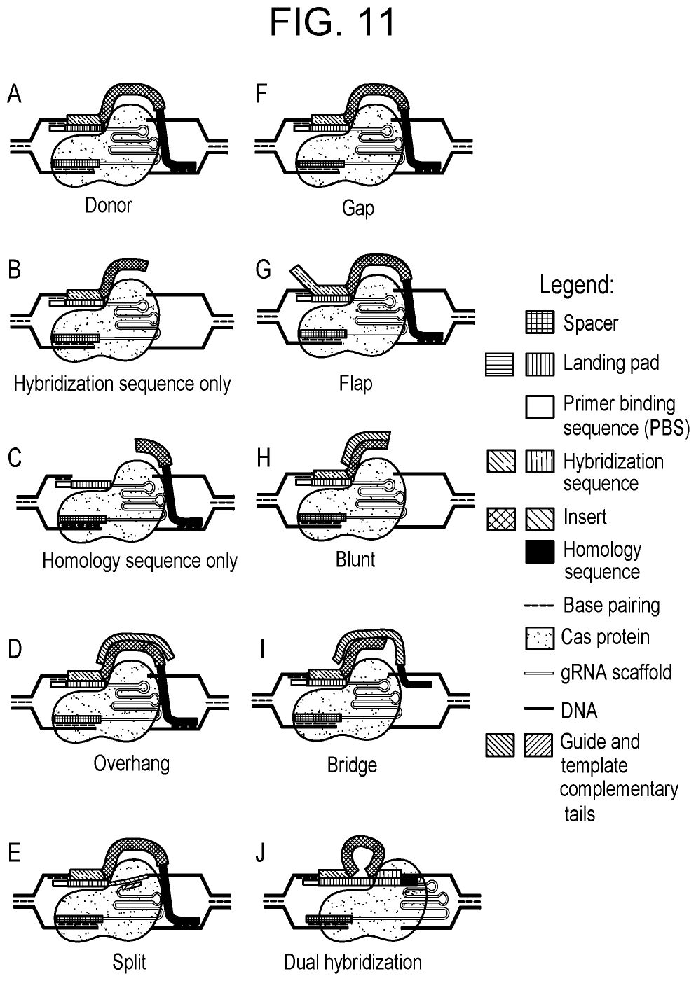

In some embodiments, the composition of the present disclosure comprises first and second polynucleotides. In some embodiments, the first polynucleotide comprises: (i) a guide sequence; (ii) a Cas-binding region; (iii) a first hybridization region; and (iv) a primer binding sequence, wherein the primer-binding sequence is at a 3′ end of the first polynucleotide; and the second polynucleotide comprises: a second polynucleotide comprising: (i) a second hybridization region that is complementary to the first hybridization region; and (ii) a sequence of interest (SOI). In some embodiments, the first polynucleotide comprises, in 5′ to 3′ order: the guide sequence, the Cas-binding region, the first hybridization region, and the primer binding sequence. In some embodiments, the second polynucleotide comprises, in 5′ to 3′ order: the second hybridization region and the SOI. In some embodiments, the first polynucleotide and/or the second polynucleotide comprises RNA, DNA, a modified nucleotide, or combination thereof. Modified nucleotides are described herein. A non-limiting, exemplary illustration of a composition comprising first and second polynucleotides as described herein is shown in FIG. 11(B).

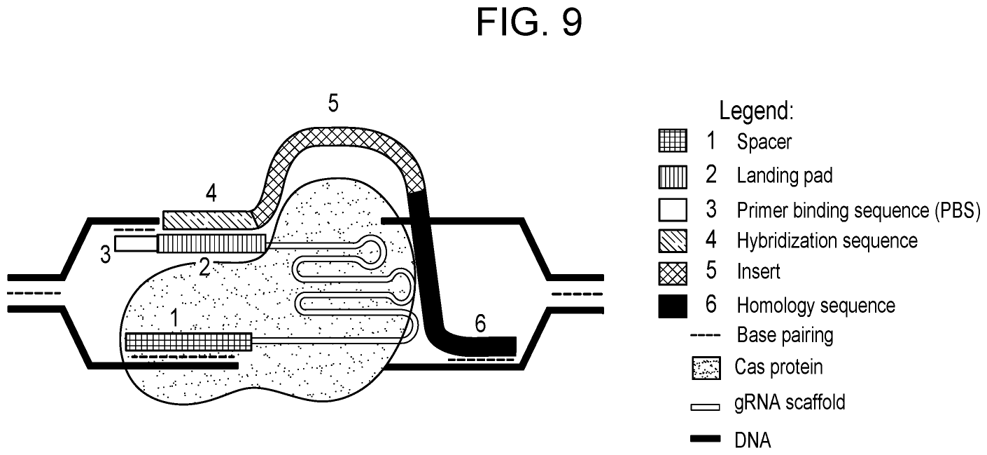

In some embodiments, the first polynucleotide and/or the second polynucleotide further comprises a homology sequence. In some embodiments, the first polynucleotide comprises, in 5′ to 3′ order: the guide sequence, the Cas-binding region, the first hybridization region, and the primer binding sequence. In some embodiments, the second polynucleotide comprises, in 5′ to 3′ order: the second hybridization region, the SOI, and the homology sequence. In some embodiments, the homology sequence is capable of hybridizing to a sequence proximal to the cleaved target polynucleotide (e.g., generated by the Cas nuclease or Cas nickase). In some embodiments, the guide sequence hybridizes to a region on one side of the cleavage site, and the homology sequence hybridizes to a region on the other side of the cleavage site, e.g., as illustrated in FIG. 9 or FIG. 11(A). In FIG. 9 and FIG. 11(A), a first polynucleotide comprises the guide sequence (referred to as “spacer”), Cas-binding region (referred to as “gRNA scaffold”), first hybridization region (referred to as “landing pad”), and primer binding sequence (PBS), a second polynucleotide comprises the second hybridization region (referred to as “hybridization sequence”), SOI (referred to as “insert”), and homology sequence.

In some embodiments, the first polynucleotide comprises, in 5′ to 3′ order: the guide sequence, the Cas-binding region, the homology sequence, the first hybridization region, and the primer binding sequence. In some embodiments, the second polynucleotide comprises, in 5′ to 3′ order: the second hybridization region, the SOI, and a further hybridization region, wherein the second hybridization region and the further hybridization region hybridize with non-overlapping portions of the first hybridization region. In some embodiments, the second hybridization region and the further hybridization region flank the SOI and hybridize with adjacent portions of the first hybridization region, e.g., as illustrated in FIG. 11(J). In some embodiments, the guide sequence hybridizes to a region on one side of the cleavage site, and the homology sequence hybridizes to a region on the other side and on the opposite DNA strand of the cleavage site, e.g., as illustrated in FIG. 11(J).

In some embodiments, the composition of the present disclosure comprises first, second, and third polynucleotides. In some embodiments, the first polynucleotide comprises: (i) a guide sequence; (ii) a Cas-binding region; and (iii) a first hybridization region, wherein the first hybridization region is at a 3′ end of the first polynucleotide; the second polynucleotide comprises: (i) a second hybridization region that is complementary to the first hybridization region; (ii) a third hybridization region; and (iii) a primer binding sequence, wherein the primer binding sequence is at a 3′ end of the second polynucleotide; and the third polynucleotide comprises: (i) a fourth hybridization region that is complementary to the third hybridization region; (ii) a sequence of interest (SOI). In some embodiments, the first polynucleotide comprises, in 5′ to 3′ order: the guide sequence, the Cas-binding region, and the first hybridization region. In some embodiments, the second polynucleotide comprises, in 5′ to 3′ order: the second hybridization region, the third hybridization region, and the primer binding sequence. In some embodiments, the third polynucleotide comprises, in 5′ to 3′ order: the fourth hybridization region and the SOI. In some embodiments, any of the first polynucleotide, the second polynucleotide, and/or the third polynucleotide comprises RNA, DNA, a modified nucleotide, or combination thereof. Modified nucleotides are described herein.

In some embodiments, the any of the first polynucleotide, the second polynucleotide, and/or the third polynucleotide further comprises a homology sequence as described herein, e.g., as illustrated in FIG. 11(E). In some embodiments, the first polynucleotide comprises, in 5′ to 3′ order: the guide sequence, the Cas-binding region, and the first hybridization region. In some embodiments, the second polynucleotide comprises, in 5′ to 3′ order: the second hybridization region, the third hybridization region, and the primer binding sequence. In some embodiments, the third polynucleotide comprises, in 5′ to 3′ order: the fourth hybridization region, the SOI, and the homology sequence.

In some embodiments, the compositions herein, e.g., comprising the first, second and/or third polynucleotides, further comprise a SOI complement oligonucleotide that comprises a sequence complementary to the SOI. In some embodiments, the SOI complement oligonucleotide is longer than the SOI at one or both of the 5′ and 3′ ends, thereby generating a double-stranded sequence comprising overhang ends, as illustrated in FIG. 11(D). In some embodiments, the SOI complement oligonucleotide is the same length as the SOI, thereby generating a double-stranded sequence with blunt ends, e.g., as illustrated in FIG. 11(H). In some embodiments, the SOI complement oligonucleotide hybridizes with the SOI to form a double-stranded SOI. In some embodiments, a double-stranded SOI has higher precise insertion efficiency as compared to a single-stranded SOI. In some embodiments, the SOI complement oligonucleotide further comprises a homology sequence, e.g., as illustrated in FIG. 11(I).

In some embodiments, the second hybridization region hybridizes over the entire length of the first hybridization region. In some embodiments, the second hybridization region is shorter than the first hybridization region, e.g., by about 1 to about 10 nucleotides, or about 2 to about 8 nucleotides, or about 3 to about 6 nucleotides, or about 4 to about 5 nucleotides. In some embodiments, the second hybridization region is shorter than the first hybridization region at a 5′ end of the second hybridization region, thereby leaving a gap between the second hybridization region and the cleaved DNA, e.g., as illustrated in FIG. 11(F). In some embodiments, the second hybridization region is longer than the first hybridization region, e.g., by about 1 to about 10 nucleotides, or about 2 to about 8 nucleotides, or about 3 to about 6 nucleotides, or about 4 to about 5 nucleotides. In some embodiments, the second hybridization region is longer than the first hybridization region at a 5′ end of the second hybridization region, thereby providing a flap following hybridization between the first and second hybridization regions, e.g., as illustrated in FIG. 11(G). In some embodiments, the flap recruits and/or engages repair machinery such as FEN1, thereby facilitating precise insertion of the sequence of interest.

In some embodiments, the disclosure provides a polynucleotide comprising: (i) an RNA guide sequence; (ii) a Cas-binding region; and (iii) a DNA template sequence, wherein the DNA template sequence is at a 3′ end of the polynucleotide. In some embodiments, the disclosure provides a polynucleotide comprising: (i) an RNA guide sequence; (ii) a Cas-binding region; and (iii) a DNA template sequence, wherein the DNA template sequence is at a 3′ end of the polynucleotide, and wherein the DNA template sequence comprises a phosphorothioate bond. In some embodiments, the polynucleotide comprises, in 5′ to 3′ order: the guide sequence (e.g., RNA guide sequence), the Cas-binding region, and the DNA template sequence.

In some embodiments, the guide sequence is capable of hybridizing with a target polynucleotide, e.g., a target polynucleotide in a genome of a host cell. In embodiments, the guide sequence is complementary to the target polynucleotide. In some embodiments, the target polynucleotide is a target DNA intended to be cleaved by the Cas nuclease or Cas nickase. In some embodiments, the guide sequence comprises RNA, i.e., an RNA guide sequence. In some embodiments, the guide sequence comprises a combination of RNA and DNA. Hybrid RNA-DNA guide sequences are further described in, e.g., Rueda et al., Nat Comm 8:1610 (2017).

In some embodiments, the guide sequence is about 10 to about 40 nucleotides in length. In some embodiments, the guide sequence is about 12 to about 30 nucleotides in length. In some embodiments, the guide sequence is about 15 to about 20 nucleotides in length. In some embodiments, the guide sequence is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In some embodiments, the guide sequence is a sufficient length for hybridizing to the target polynucleotide.

In some embodiments, the Cas-binding region is capable of binding to the Cas protein (e.g., Cas nuclease or Cas nickase) in a composition, thereby forming a complex with the Cas protein. In some embodiments, the Cas-binding region comprises RNA. In some embodiments, the Cas-binding region comprises a combination of RNA and DNA. Hybrid RNA-DNA sequences that can bind to and/or activate Cas proteins are further described in, e.g., Rueda et al., Nat Comm 8:1610 (2017).

In some embodiments, the Cas-binding region comprises a tracrRNA that binds to and activates the Cas protein. In some embodiments, the Cas-binding region is capable of hybridizing with a tracrRNA, and the composition further comprises a tracrRNA. In some embodiments, the tracrRNA is capable of binding the Cas nuclease or Cas nickase. In some embodiments, the tracrRNA is capable of activating the Cas nuclease or Cas nickase. In some embodiments, the activating comprises initiating or increasing the cleavage activity of the Cas nuclease or Cas nickase. In some embodiments, the activating comprises promoting binding of the Cas nuclease or Cas nickase to a target polynucleotide (e.g., as guided by the guide sequence). In some embodiments, the activating comprises a combination of promoting binding of the Cas nuclease or Cas nickase to the target polynucleotide; and initiating or increasing cleavage activity of the Cas nuclease or Cas nickase. TracrRNA sequences of Cas proteins (e.g., Cas9, Cas12a, or Type II-B Cas proteins described herein) are available from public databases, including RNAcentral and Rfam, and further described in, e.g., Chylinski et al., RNA Biol 10(5):726-737 (2013) and Gasiunas et al., Nat Comm 11:5512 (2020).

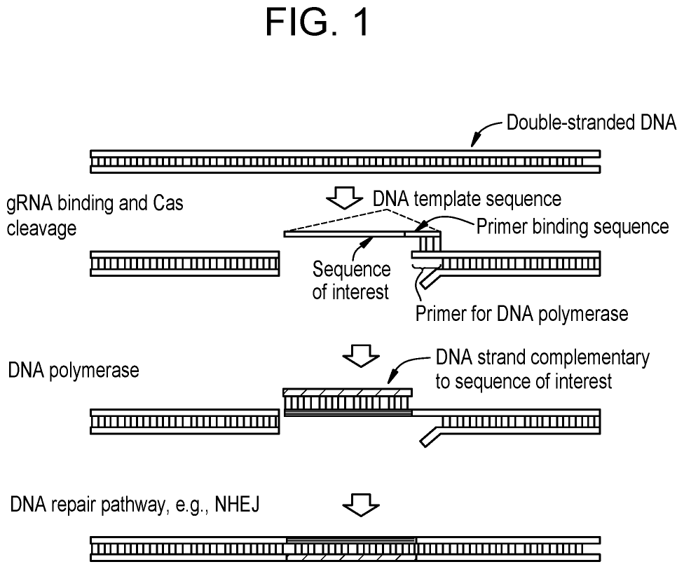

In some embodiments, the polynucleotide of the disclosure comprises a DNA template sequence at a 3′ end of the polynucleotide. In some embodiments, the DNA template sequence comprises single-stranded DNA. In some embodiments, the DNA template sequence comprises a sequence of interest. In some embodiments, the DNA template sequence comprises a primer binding sequence and a sequence of interest. In some embodiments, the DNA template sequence comprises a template for amplification by a DNA polymerase. In some embodiments, the sequence of interest comprises a template for amplification by a DNA polymerase. In some embodiments, the Cas nuclease or Cas nickase of the composition is guided to a target polynucleotide by the guide sequence and cleaves the target polynucleotide, and one strand of the cleaved target polynucleotide hybridizes to the primer binding sequence and serves as a primer for a DNA polymerase. In some embodiments, the DNA polymerase is capable of synthesizing a DNA strand complementary to the sequence of interest to form a double-stranded sequence comprising the sequence of interest. In some embodiments, the double-stranded sequence comprising the sequence of interest is inserted into the cleaved target polynucleotide, e.g., via ligation or a DNA repair pathway described herein. An exemplary, non-limiting outline of a Cas-mediated targeted insertion of a sequence of interest is illustrated in FIG. 1. In FIG. 1, a Cas nuclease is guided to a target DNA in a cell by a guide sequence and cleaves the target DNA. The DNA template sequence comprises (i) a primer binding sequence that hybridizes with the cleaved DNA, which serves as a primer, and (ii) a sequence of interest. A DNA polymerase, e.g., endogenous DNA polymerase of the cell, binds to the primer and synthesizes a DNA strand complementary to the sequence of interest, thereby forming a double-stranded sequence comprising the sequence of interest. The double-stranded sequence can be inserted into the cleaved target DNA by a DNA repair pathway, e.g., NHEJ.

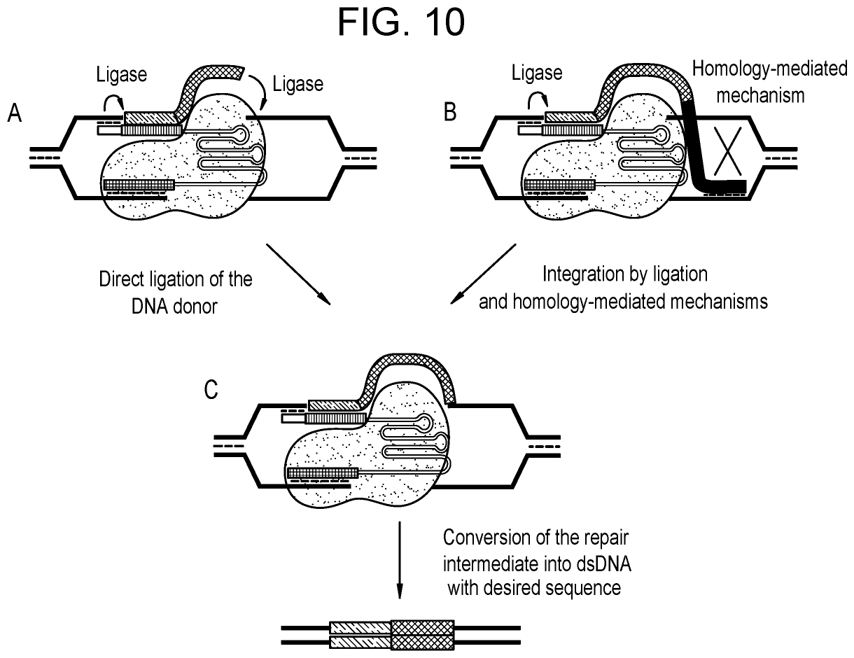

In some embodiments, components of the DNA template sequence described herein, e.g., the sequence of interest and the primer binding sequence, are located on two separate polynucleotides, e.g., first and second polynucleotides as described herein. An exemplary, non-limiting outline of an embodiment in which the sequence of interest and the primer binding sequence are located on two separate polynucleotides is illustrated in FIGS. 10A-10C. In FIG. 10A, the guide sequence, Cas-binding region, first hybridization region, and primer binding site are located on a first polynucleotide. A double-stranded break is generated at a target DNA by a Cas nuclease guided to the target DNA by a guide sequence. The primer binding site hybridizes to the cleaved DNA, and a second polynucleotide hybridizes to the first polynucleotide via the first and second hybridization regions that comprise complementary sequences. The second polynucleotide comprises a sequence of interest, which is ligated into the cleaved DNA at both the 5′ and 3′ ends by a ligase (FIG. bOA). The second polynucleotide can further include a homology sequence, and the 5′ end is ligated into the cleaved DNA by a ligase, and the 3′ end is integrated by a homology-mediated mechanism (FIG. 10B). In some embodiments, the homology-mediated mechanism comprises HDR, Synthesis-dependent strand annealing (SDSA), Single-stranded annealing (SSA), alternative end joining (alt-EJ), or combination thereof. Following the ligation and/or homology-mediated integration, the complex is converted into double-stranded DNA, thereby integrating the sequence of interest into the target DNA (FIG. 10C).

In some embodiments, the sequence of interest comprises a gene of interest. As used herein, the term “gene of interest” refers to a gene that encodes a biomolecule of interest (e.g., a protein or an RNA molecule). In some embodiments, the gene of interest encodes a protein of interest. In some embodiments, the protein of interest comprises an intracellular protein, a membrane protein, an extracellular protein, or combination thereof. In some embodiments, the protein of interest comprises a nuclear protein, a transcription factor, a nuclear membrane transporter, an intracellular organelle associated protein, a membrane receptor, a catalytic protein, an enzyme, a therapeutic protein, a membrane protein, a membrane transport protein, a signal transduction protein, an immunological protein, or combination thereof. In some embodiments, the immunological protein comprises an antibody, e.g., IgG, IgA, IgM, IgD, IgE, or combination thereof. In some embodiments, the sequence of interest encodes a copy of a native gene of the host cell. In some embodiments, the sequence of interest encodes a copy of a native gene that is deficient in the host cell. In some embodiments, the host cell comprises a mutation in a gene, and the sequence of interest encodes a wild-type copy of the gene. In some embodiments, the host cell comprises a wild-type gene, and the sequence of interest encodes a copy of the gene comprising a mutation of interest. In some embodiments, the sequence of interest encodes a heterologous gene that is not naturally occurring in the host cell.

In some embodiments, the gene of interest encodes an RNA of interest. In some embodiments, the RNA of interest comprises a therapeutic RNA. In some embodiments, the RNA of interest comprises messenger RNA (mRNA), transfer RNA (tRNA), ribosomal RNA (rRNA), small nuclear RNA (snRNA), antisense RNA, microRNA (miRNA), small interfering RNA (siRNA), cell-free RNA (cfRNA), or combination thereof. In some embodiments, the sequence of interest comprises a regulatory element of interest. In some embodiments, the sequence of interest is inserted into a target polynucleotide of a host cell, such that the regulatory element on the sequence of interest is capable of regulating a native gene of the host cell. Regulatory elements are described herein and include, e.g., promoters, enhancers, silencers, operators, response elements, 5′ UTR, 3′ UTR, insulators, and the like.

In some embodiments, the DNA template sequence is about 5 nucleotides to about 5000 nucleotides in length. In some embodiments, the DNA template sequence is about 6 nucleotides to about 1000 nucleotides in length. In some embodiments, the DNA template sequence is about 7 nucleotides to about 750 nucleotides in length. In some embodiments, the DNA template sequence is about 8 nucleotides to about 500 nucleotides in length. In some embodiments, the DNA template sequence is about 9 nucleotides to about 250 nucleotides in length. In some embodiments, the DNA template sequence is about 10 nucleotides to about 100 nucleotides in length. In some embodiments, the DNA template sequence is about 15 nucleotides to about 90 nucleotides in length. In some embodiments, the DNA template sequence is about 20 nucleotides to about 80 nucleotides in length. In some embodiments, the DNA template sequence is about 25 nucleotides to about 70 nucleotides in length. In some embodiments, the DNA template sequence is about 30 nucleotides to about 50 nucleotides in length. In some embodiments, the DNA template sequence is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the DNA template sequence is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the primer-binding sequence is about 3 to about 50 nucleotides in length. In some embodiments, the primer-binding sequence is about 4 to about 45 nucleotides in length. In some embodiments, the primer-binding sequence is about 5 to about 40 nucleotides in length. In some embodiments, the primer-binding sequence is about 6 to about 35 nucleotides in length. In some embodiments, the primer-binding sequence is about 7 to about 30 nucleotides in length. In some embodiments, the primer-binding sequence is about 8 to about 25 nucleotides in length. In some embodiments, the primer-binding sequence is about 10 to about 20 nucleotides in length. In some embodiments, the primer-binding sequence is about 4 to about 30 nucleotides in length. In some embodiments, the primer-binding sequence is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the primer-binding sequence is of sufficient length to hybridize with a region of the cleaved target DNA sequence.

In some embodiments, the sequence of interest is about 3 to about 500 nucleotides in length. In some embodiments, the sequence of interest is about 4 to about 100 nucleotides in length.

In some embodiments, the sequence of interest is about 5 to about 90 nucleotides in length. In some embodiments, the sequence of interest is about 6 to about 80 nucleotides in length. In some embodiments, the sequence of interest is about 7 to about 70 nucleotides in length. In some embodiments, the sequence of interest is about 8 to about 60 nucleotides in length. In some embodiments, the sequence of interest is about 9 to about 50 nucleotides in length. In some embodiments, the sequence of interest is about 10 to about 40 nucleotides in length. In some embodiments, the sequence of interest is about 11 to about 30 nucleotides in length. In some embodiments, the sequence of interest is about 12 to about 20 nucleotides in length. In some embodiments, the sequence of interest is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides in length. In some embodiments, the sequence of interest is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the DNA template sequence comprises a modified nucleotide, a non-B DNA structure, a DNA polymerase recruitment moiety, a DNA ligase recruitment moiety, or a combination thereof.

In some embodiments, the DNA template sequence comprises a modified nucleotide. In some embodiments, the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

In some embodiments, the modified nucleotide reduces or prevents overextension of the sequence of interest by the DNA polymerase. In some embodiments, reducing or preventing overextension of the sequence of interest by the DNA polymerase increases the precision of inserting the double-stranded sequence comprising the sequence of interest. In some embodiments, the modified nucleotide comprises an abasic site, also known as an apurinic/apyrimidinic (AP site). In some embodiments, the modified nucleotide comprises a covalent linker. In some embodiments, the covalent linker comprises a triethylene glycol (TEG) linker. In some embodiments, the covalent linker comprises an amino linker. TEG linkers and amino linkers have been shown to block polymerase extension; see, e.g., Strobel et al., bioRxiv doi:10.1101/2019.12.26.888743 (23 Jan. 2020).

In some embodiments, the modified nucleotide reduces or prevents nuclease degradation of the polynucleotide of the disclosure. In some embodiments, the modified nucleotide comprises a xeno nucleic acid (XNA). An XNA is a synthetic nucleotide analogue that has a different sugar group than the deoxyribose of DNA or the ribose of RNA. Exemplary sugar groups for XNA include, but are not limited to, threose, cyclohexene, glycol, or a locked ribose. In some embodiments, the XNA comprises 1,5-anhydrohexitol nucleic acid (HNA), cyclohexene nucleic acid (CeNA), threose nucleic acid (TNA), glycol nucleic acid (GNA), locked nucleic acid (LNA), and peptide nucleic acid (PNA). In some embodiments, the modified nucleotide comprises a locked nucleic acid (LNA), also known as a bridged nucleic acid (BNA). An LNA is a modified RNA nucleotide in which the ribose moiety is modified with an extra bridge connecting the 2′ oxygen and 4′ carbon. In some embodiments, the modified nucleotide comprises a peptide nucleic acid (PNA). Unlike the deoxyribose or ribose backbones of DNA or RNA, the backbone of a PNA polymer comprises N-(2-aminoethyl)-glycine units linked by peptide bonds, and the purine and pyrimidine bases are linked to the PNA backbone by a methylene bridge and a carbonyl group. In some embodiments, the modified nucleotide comprises a phosphorothioate bond. A phosphorothioate bond comprises a sulfur atom in place of one of the oxygens in the phosphate group linking two nucleotides. In some embodiments, the presence of an XNA, e.g., an LNA or a PNA, or a phosphorothioate bond in a polynucleotide increases stability of the polynucleotide against nuclease degradation.

In some embodiments, the presence of a modified nucleotide in a polynucleotide (e.g., the polynucleotide of the composition provided herein) is capable of recruiting a DNA polymerase to the polynucleotide. In some embodiments, recruiting a DNA polymerase comprises: increasing the likelihood that a DNA polymerase recognizes the polynucleotide, e.g., due to presence of the modified nucleotide therein; promoting binding of a DNA polymerase to the polynucleotide; and/or activating a DNA polymerase, e.g., initiating or increasing activity of the DNA polymerase. In some embodiments, the recruited DNA polymerase binds to a strand of the cleaved target polynucleotide and extends the sequence of interest on the DNA template sequence, as described herein.

In some embodiments, the modified nucleotide comprises a DNA lesion. As used herein, a “DNA lesion” refers to a region of a DNA polynucleotide containing a base alteration, base deletion, and/or sugar alteration typically indicative of DNA damage. DNA lesions can be caused by hydrolysis, oxidation, alkylation, depurination, depyrimidination, and/or deamination of a nucleobase. In some embodiments, the DNA lesion is capable of recruiting a DNA polymerase. In some embodiments, the DNA lesion comprises 8-oxoguanine, thymine-glycol, N7-(2-hydroxethyl) guanine (7HEG), 7-(2-oxoethyl)guanine, or a combination thereof. In some embodiments, the DNA lesion comprises 8-oxoguanine, thymine-glycol, or a combination thereof.

In some embodiments, the modified nucleotide comprises a DNA photoproduct. DNA photoproducts are ultraviolet (UV)-induced DNA lesions and are further described in, e.g., Yokoyama et al., Int J Mol Sci 15(11):20321-20338 (2014). In some embodiments, the DNA photoproduct is capable of recruiting a DNA polymerase. In some embodiments, the DNA photoproduct comprises a pyrimidine dimer, a cyclobutane pyrimidine dimer (CPD), a pyrimidine (6-4) pyrimidone photoproduct (also referred to as a “(6-4) photoproduct”), an adenine-thymine heterodimer, a Dewar pyrimidinone, or a combination thereof. In some embodiments, the DNA photoproduct comprises CPD, a (6-4) photoproduct, or a combination thereof.

In some embodiments, the modified nucleotide comprises a modified deoxyribonucleoside. In some embodiments, the modified deoxyribonucleoside is capable of recruiting a DNA polymerase. In some embodiments, the modified deoxyribonucleoside comprises a base not typically present in DNA, i.e., adenine, cytosine, guanine, or thymine. In some embodiments, the modified deoxyribonucleoside comprises deoxyuridine, acrolein-deoxyguanine, malondialdehyde-deoxyguanine, deoxyinosine, deoxyxanthosine, or a combination thereof. In some embodiments, the modified deoxyribonucleoside comprises deoxyuridine.

In some embodiments, the modified nucleotide comprises a methylated nucleotides. In some embodiments, methylated nucleotides, e.g., methylated cytosines, are capable of recruiting a DNA polymerase. In some embodiments, the methylated nucleotide comprises 5-hydroxymethylcytosine, 5-methylcytosine, or a combination thereof.

In some embodiments, the DNA template sequence comprises a non-B DNA structure. As used herein, “a non-B DNA structure” is a DNA secondary structural conformation that is not the canonical right-handed B-DNA helix. Non-limiting examples of non-B DNA structures include G-quadruplex, triplex DNA (H-DNA), Z-DNA, cruciform, slipped DNA strands, A-tract bending, sticky DNA. Non-B DNA structures are further described in, e.g., Guiblet et al., Nucleic Acids Res 49(3):1497-1516 (2021). In some embodiments, the non-B DNA structure is capable of recruiting a DNA polymerase. In some embodiments, the non-B DNA structure comprises a hairpin, a cruciform, Z-DNA, H-DNA (triplex DNA), G-quadruplex DNA (tetraplex DNA), slipped DNA, sticky DNA, or a combination thereof.

In some embodiments, the DNA template sequence comprises a DNA polymerase recruitment moiety. DNA polymerase recruitment is described herein. Non-limiting examples of DNA polymerases that can be recruited by the DNA polymerase recruitment moiety include bacterial DNA polymerases such as Pol I (including a Klenow fragment thereof), Pol II, Pol III, Pol IV, or Pol V; eukaryotic DNA polymerases such as Pol α, Pol β, Pol λ, Pol γ, Pol σ, Pol μ, Pol δ, Pol ε, Pol η, Pol ι, Pol κ, Pol ζ, Pol θ, REV1, or REV3; isothermal DNA polymerases such as Bst, T4, or Φ29 (phi29) DNA polymerase; thermostable DNA polymerases such as Taq, Pfu, KOD, Tth, or Pwo DNA polymerase; or a variant or homologue thereof.

In some embodiments, the DNA polymerase recruitment moiety comprises a modified nucleotide, e.g., a DNA lesion, DNA photoproduct, modified deoxyribonucleoside, and/or methylated nucleotide described herein. In some embodiments, the modified nucleotide recruits a translesion DNA synthesis (TLS) polymerase. TLS polymerases are capable of extending DNA that comprises a modified nucleotide described herein. Exemplary TLS polymerases include, but are not limited to, Pol II, Pol IV, and Pol V from E. coli; Rev1p, Rev3p, and Pol η from S. cerevisiae; and human REV1, REV3, Pol η, Pol ι, and Pol κ. Further TLS polymerases are described in, e.g., Goodman et al., Cold Spring Harb Perspect Biol 5(10): a010363 (2013) and Waters et al., Microbiol Mol Biol Rev 73(1):134-154 (2009). In some embodiments, the DNA polymerase recruitment moiety comprises a non-B DNA structure described herein. In some embodiments, the DNA polymerase recruited by the non-B DNA structure is capable of extending DNA through the non-B DNA structure. In some embodiments, the non-B DNA structure recruits a DNA polymerase selected from PrimPol, REV1, REV3, Pol δ, Pol η, Pol ι, Pol κ, and Pol θ.

In some embodiments, the DNA polymerase recruitment moiety comprises a DNA polymerase recruitment protein. In some embodiments, the DNA polymerase recruitment protein is capable of recognizing and binding a DNA polymerase. In some embodiments, the DNA polymerase recruitment protein is linked to the DNA template sequence. In some embodiments, the DNA polymerase recruitment protein is cross-linked to the primer binding sequence. Methods of linking proteins to polynucleotides are known to one of ordinary skill in the art and include, for example, covalent conjugation methods such as copper-catalyzed cycloaddition, strain-promoted azide-alkyne cycloaddition, and inverse-electron-demand Diels-Alder reaction (e.g., reaction between a cyclopropene and a tetrazine); or affinity-based methods, e.g., linking a polynucleotide comprising a biotin moiety with a protein comprising an avidin or streptavidin moiety.

In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

In some embodiments, the DNA template sequence comprises a DNA ligase recruitment moiety. In some embodiments, the presence of a DNA ligase recruitment moiety in a polynucleotide (e.g., the polynucleotide of the composition provided herein) is capable of recruiting a DNA ligase to the polynucleotide. In some embodiments, recruiting a DNA ligase comprises: increasing the likelihood that a DNA ligase recognizes the polynucleotide, e.g., due to presence of the DNA ligase recruitment moiety therein; promoting binding of a DNA ligase to the polynucleotide; and/or activating a DNA ligase, e.g., initiating or increasing activity of the DNA ligase. In some embodiments, the recruited DNA ligase binds to a double-stranded sequence comprising the sequence of interest generated by a DNA polymerase and ligates the double-stranded sequence into the cleaved target polynucleotide, as described herein.

In some embodiments, the DNA ligase recruitment moiety comprises a 5′ adenylation of the DNA template sequence. In some embodiments, the DNA ligase recruitment moiety comprises a DNA ligase recruitment protein. Exemplary DNA ligase recruitment proteins include, but are not limited to, DNA-dependent protein kinase (DNA-PK), proliferating cell nuclear antigen (PCNA), or X-ray repair cross-complementing protein 1 (XRCC1). Non-limiting examples of DNA ligases that can be recruited by the DNA ligase moiety include bacterial DNA ligases such as E. coli DNA ligase, T4 DNA ligase, T7 DNA ligase, mammalian DNA ligases such as DNA ligase I, DNA ligase II, DNA ligase III, or DNA ligase IV, thermostable DNA ligases such as Taq DNA ligase, or a variant or homologue thereof.

In some embodiments, the guide sequence, Cas-binding region, and DNA template sequence described herein are present on a single polynucleotide in the composition. In some embodiments, the DNA template sequence is positioned 3′ of the guide sequence and the Cas-binding region. In some embodiments, the DNA template sequence is at a 3′ end of the polynucleotide. In some embodiments, the DNA template sequence being positioned at a 3′ end of the polynucleotide facilitates binding and/or extension of the cleaved target polynucleotide by a DNA polymerase to form a double-stranded sequence comprising the sequence of interest, as described herein. In some embodiments, the DNA template sequence being positioned at a 3′ end of the polynucleotide facilitates ligation of the double-stranded sequence into the cleaved target polynucleotide by a DNA ligase, as described herein.

In some embodiments, the guide sequence, Cas-binding region, and DNA template sequence described herein are present on more than one polynucleotide in the composition. In some embodiments, the guide sequence and Cas-binding region are on a first polynucleotide, and the DNA template sequence is on a second polynucleotide. In some embodiments, the guide sequence is on a first polynucleotide, and the Cas-binding region and the DNA template sequence are on a second polynucleotide. In some embodiments, the first polynucleotide comprises a first hybridization region. In some embodiments, the second polynucleotide comprises a second hybridization region that is complementary to the first hybridization region. In some embodiments, the first hybridization region and the second hybridization region are capable of hybridizing. In some embodiments, the first hybridization region and the second hybridization region comprise RNA. In some embodiments, the first hybridization region and the second hybridization region comprise single-stranded DNA. In some embodiments, the first hybridization region comprises RNA, and the second hybridization region comprises single-stranded DNA. In some embodiments the first hybridization region comprises single-stranded DNA, and the second hybridization region comprises RNA. In some embodiments, the RNA and single-stranded DNA are capable of hybridizing.

In some embodiments, the first hybridization region is at a 3′ end of the first polynucleotide. In some embodiments, the second hybridization region is at a 5′ end of the second polynucleotide. In some embodiments, upon hybridization of the first hybridization region to the second hybridization region, the DNA template sequence is positioned 3′ of both the guide sequence and the Cas-binding region. In some embodiments, the DNA template sequence is at a 3′ end of the second polynucleotide. In some embodiments, the DNA template sequence being positioned at a 3′ end of the second polynucleotide facilitates binding and/or extension of the cleaved target polynucleotide by a DNA polymerase to form a double-stranded sequence comprising the sequence of interest, as described herein. In some embodiments, the DNA template sequence being positioned at a 3′ end of the second polynucleotide facilitates ligation of the double-stranded sequence into the cleaved target polynucleotide by a DNA ligase, as described herein.

In some embodiments, the guide sequence, the Cas-binding region, the sequence of interest (SOI), and the primer binding sequence are present on more than one polynucleotide, as described herein. In some embodiments, the guide sequence, the Cas-binding region, and the primer binding sequence are on a first polynucleotide; the SOI is on a second polynucleotide; and the first and second polynucleotides respectively comprise first and second hybridization regions that are capable of hybridizing to each other. In some embodiments, the primer binding sequence is positioned at a 3′ end of the first polynucleotide. In some embodiments, the primer binding sequence being positioned at a 3′ end of the first polynucleotide facilitates binding and/or ligation of the SOI into the cleaved target polynucleotide by a DNA ligase, as described herein. In some embodiments, the primer binding sequence being positioned at a 3′ end of the first polynucleotide facilitates binding and/or extension of the cleaved target polynucleotide by a DNA polymerase to form a double-stranded sequence comprising the SOI, as described herein.

In some embodiments, the first hybridization region is about 3 to about 10000 nucleotides in length. In some embodiments, the first hybridization region is about 4 to about 5000 nucleotides in length. In some embodiments, the first hybridization region is about 5 to about 1000 nucleotides in length. In some embodiments, the first hybridization region is about 10 to about 800 nucleotides in length. In some embodiments, the first hybridization region is about 20 to about 600 nucleotides in length. In some embodiments, the first hybridization region is about 30 to about 500 nucleotides in length. In some embodiments, the first hybridization region is about 40 to about 400 nucleotides in length. In some embodiments, the first hybridization region is about 50 to about 300 nucleotides in length. In some embodiments, the first hybridization region is about 100 to about 200 nucleotides in length. In some embodiments the first hybridization region is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, or 5000 nucleotides in length.

In some embodiments, the second hybridization region is about 3 to about 10000 nucleotides in length. In some embodiments, the second hybridization region is about 4 to about 5000 nucleotides in length. In some embodiments, the second hybridization region is about 5 to about 1000 nucleotides in length. In some embodiments, the second hybridization region is about 10 to about 800 nucleotides in length. In some embodiments, the second hybridization region is about 20 to about 600 nucleotides in length. In some embodiments, the second hybridization region is about 30 to about 500 nucleotides in length. In some embodiments, the second hybridization region is about 40 to about 400 nucleotides in length. In some embodiments, the second hybridization region is about 50 to about 300 nucleotides in length. In some embodiments, the second hybridization region is about 100 to about 200 nucleotides in length. In some embodiments the second hybridization region is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, or 5000 nucleotides in length.

In some embodiments, the first and second hybridization regions are substantially the same length. In some embodiments, the first and second hybridization regions differ in length by less than about 1, less than about 2, less than about 3, less than about 4, less than about 5, less than about 6, less than about 7, less than about 8, less than about 9, less than about 10, less than about 15, less than about 20, less than about 25, less than about 30, less than about 35, less than about 40, less than about 45, less than about 50, less than about 55, less than about 60, less than about 65, less than about 70, less than about 75, less than about 80, less than about 85, less than about 90, less than about 95, or less than about 100 nucleotides. In some embodiments, the first and second hybridization regions differ in length by less than about 30%, less than about 25%, less than about 20%, less than about 15%, less than about 10%, less than about 5%, less than about 2%, or less than about 1% of the number of nucleotides in the first or second hybridization regions. In some embodiments, the first and second hybridization regions are identical in length. In some embodiments, the first and second hybridization regions are fully complementary. In some embodiments, the first and second hybridization regions are at least about 80%, at least about 85%, at least about 90%, at least about 95%, at least about 96%, at least about 97%, at least about 98%, or at least about 99% complementary. In some embodiments, the first and second hybridization regions are capable of hybridizing under standard nucleic acid hybridization conditions, e.g., as described in Herzer and Englert, “Chapter 14. Nucleic Acid Hybridization,” in Molecular Biology Problem Solver: A Laboratory Guide, (2001) ed. Alan S. Gersterin; Wiley-Liss, Inc.

In some embodiments, the guide sequence is on a first polynucleotide, the Cas-binding region is on a second polynucleotide, and the DNA template sequence is on a third polynucleotide. In some embodiments, the first polynucleotide comprises a first hybridization region at a 3′ end; the second polynucleotide comprises (i) a second hybridization region at a 5′ end that is complementary to the first hybridization region and (ii) a third hybridization region at a 3′ end that is complementary to a fourth hybridization region; and the third polynucleotide comprises the fourth hybridization region at a 5′ end. In some embodiments, the second polynucleotide comprises a first hybridization region at a 3′ end; the first polynucleotide comprises (i) a second hybridization region at a 5′ end that is complementary to the first hybridization region and (ii) a third hybridization region at a 3′ end that is complementary to a fourth hybridization region; and the third polynucleotide comprises the fourth hybridization region at a 5′ end. In some embodiments, upon hybridization of the first, second, third, and fourth hybridization regions, the DNA template sequence is positioned 3′ of both the guide sequence and the Cas-binding region. In some embodiments, each of the first, second, third, and fourth hybridization regions is about 3 to about 10000 nucleotides in length, or about 4 to about 5000 nucleotides in length, or about 5 to about 1000 nucleotides in length, or about 10 to about 800 nucleotides in length, or about 20 to about 600 nucleotides in length, or about 30 to about 500 nucleotides in length, or about 40 to about 400 nucleotides in length, or about 50 to about 300 nucleotides in length, or about 100 to about 200 nucleotides in length. In some embodiments, the first and second hybridization regions are substantially the same length, and the third and fourth hybridization regions are substantially the same length. In some embodiments, the first and second hybridization regions are at least about 80%, at least about 85%, at least about 90%, at least about 95%, at least about 96%, at least about 97%, at least about 98%, at least about 99%, or 100% complementary. In some embodiments, the third and fourth hybridization regions are at least about 80%, at least about 85%, at least about 90%, at least about 95%, at least about 96%, at least about 97%, at least about 98%, at least about 99%, or 100% complementary.

In some embodiments, the guide sequence, the Cas-binding region, the SOI, and the primer binding sequence are present on first, second and third polynucleotides, as described herein. In some embodiments, the guide sequence and the Cas-binding region are present on the first polynucleotide; the primer binding sequence is on the second polynucleotide; and the SOI is on the third polynucleotide; and wherein the first and second polynucleotides respectively comprise first and second hybridization regions that are capable of hybridizing to each other, and the second and third polynucleotides respectively comprise third and fourth hybridization regions that are capable of hybridizing to each other. In some embodiments, the primer binding sequence is positioned at a 3′ end of the second polynucleotide. In some embodiments, the primer binding sequence being positioned at a 3′ end of the second polynucleotide facilitates binding and/or ligation of the SOI into the cleaved target polynucleotide by a DNA ligase, as described herein. In some embodiments, the primer binding sequence being positioned at a 3′ end of the first polynucleotide facilitates binding and/or extension of the cleaved target polynucleotide by a DNA polymerase to form a double-stranded sequence comprising the SOI, as described herein. In some embodiments, each of the first, second, third, and fourth hybridization regions is about 3 to about 10000 nucleotides in length, or about 4 to about 5000 nucleotides in length, or about 5 to about 1000 nucleotides in length, or about 10 to about 800 nucleotides in length, or about 20 to about 600 nucleotides in length, or about 30 to about 500 nucleotides in length, or about 40 to about 400 nucleotides in length, or about 50 to about 300 nucleotides in length, or about 100 to about 200 nucleotides in length. In some embodiments, the first and second hybridization regions are substantially the same length, and the third and fourth hybridization regions are substantially the same length. In some embodiments, the first and second hybridization regions are at least about 80%, at least about 85%, at least about 90%, at least about 95%, at least about 96%, at least about 97%, at least about 98%, at least about 99%, or 100% complementary. In some embodiments, the third and fourth hybridization regions are at least about 80%, at least about 85%, at least about 90%, at least about 95%, at least about 96%, at least about 97%, at least about 98%, at least about 99%, or 100% complementary.

In some embodiments, the polynucleotide of the disclosure further comprises a spacer positioned 5′ of the DNA template sequence. In some embodiments, the polynucleotide comprises the guide sequence, the Cas-binding region, and the DNA template sequence, and the spacer is positioned between the Cas-binding region and the DNA template sequence. In some embodiments, the second polynucleotide of the disclosure further comprises a spacer positioned 5′ of the DNA template sequence. In some embodiments, the second polynucleotide comprises the second hybridization region, the Cas-binding region, and the DNA template sequence, and the spacer is positioned between the Cas-binding region and the DNA template sequence. In some embodiments, the second polynucleotide comprises the second hybridization region and the DNA template sequence, and the spacer is positioned between the second hybridization region and the DNA template sequence.

In some embodiments, the spacer comprises a stop sequence for the DNA polymerase, such that the DNA polymerase are stopped after synthesizing a complementary strand of the sequence of interest. In some embodiments, the spacer comprises more than one stop sequence. In some embodiments, the spacer comprises 1, 2, 3, 4, 5, or more than 5 stop sequences. In some embodiments, multiple stop sequences provide redundancy in stopping the DNA polymerase. In some embodiments, the stop sequence inhibits the activity of the DNA polymerase. In some embodiments, the stop sequence promotes dissociation of the DNA polymerase from the DNA template sequence.

In some embodiments, the stop sequence comprises a secondary structure. In some embodiments, the secondary structure is an inhibitor of DNA polymerase activity. In some embodiments, the secondary structure promotes dissociation of the DNA polymerase from the DNA template sequence. In some embodiments, the secondary structure is a hairpin loop (also known as a stem loop). In some embodiments, the secondary structure is a pseudoknot.

In some embodiments, the spacer is about 5 to about 500 nucleotides in length. In some embodiments, the spacer is about 10 to about 400 nucleotides in length. In some embodiments, the spacer is about 10 to about 300 nucleotides in length. In some embodiments, the spacer is about 10 to about 200 nucleotides in length. In some embodiments, the spacer is about 20 to about 150 nucleotides in length. In some embodiments, the spacer is about 30 to about 100 nucleotides in length. In some embodiments, the spacer is about 50 to about 100 nucleotides in length. In some embodiments, the spacer is about 5, about 10, about 15, about 20, about 25, about 30, about 35, about 40, about 45, about 50, about 75, about 100, about 110, about 120, about 130, about 140, about 150, about 160, about 170, about 180, about 190, or about 200 nucleotides in length.

The disclosure herein envisions that elements and features of the polynucleotide as described herein can be combined in any combination. For exemplary purposes only, the polynucleotide can include the features as shown in Table 1 (from 5′ to 3′):

TABLE 1

| | | |
|---|---|---|
| RNA guide | Cas binding region | DNA template sequence |
| sequence | comprising RNA | |
| RNA guide | Cas nuclease binding region | DNA template sequence |
| sequence | comprising RNA | |
| RNA guide | Cas nickase binding region | DNA template sequence |
| sequence | comprising RNA | |
| RNA guide | Cas binding region | DNA template sequence |
| sequence | comprising DNA | |
| RNA guide | Cas nuclease binding region | DNA template sequence |
| sequence | comprising DNA | |
| RNA guide | Cas nickase binding region | DNA template sequence |
| sequence | comprising DNA | |
| RNA guide | Cas binding region | DNA template sequence comprising a |
| sequence | comprising RNA | primer binding sequence (PBS) |
| RNA guide | Cas nuclease binding region | DNA template sequence comprising a PBS |
| sequence | comprising RNA | |
| RNA guide | Cas nickase binding region | DNA template sequence comprising a PBS |
| sequence | comprising RNA | |
| RNA guide | Cas binding region | DNA template sequence comprising a PBS |
| sequence | comprising DNA | |
| RNA guide | Cas nuclease binding region | DNA template sequence comprising a PBS |
| sequence | comprising DNA | |
| RNA guide | Cas nickase binding region | DNA template sequence comprising a PBS |
| sequence | comprising DNA | |
| RNA guide | Cas binding region | DNA template sequence comprising a |
| sequence | comprising RNA | sequence of interest (SOI) |
| RNA guide | Cas nuclease binding region | DNA template sequence comprising a SOI |
| sequence | comprising RNA | |
| RNA guide | Cas nickase binding region | DNA template sequence comprising a SOI |
| sequence | comprising RNA | |
| RNA guide | Cas binding region | DNA template sequence comprising a SOI |
| sequence | comprising DNA | |
| RNA guide | Cas nuclease binding region | DNA template sequence comprising a SOI |
| sequence | comprising DNA | |
| RNA guide | Cas nickase binding region | DNA template sequence comprising a SOI |
| sequence | comprising DNA | |
| RNA guide | Cas binding region | DNA template sequence comprising a PBS |
| sequence | comprising RNA | and a SOI |
| RNA guide | Cas nuclease binding region | DNA template sequence comprising a PBS |
| sequence | comprising RNA | and a SOI |
| RNA guide | Cas nickase binding region | DNA template sequence comprising a PBS |
| sequence | comprising RNA | and a SOI |
| RNA guide | Cas binding region | DNA template sequence comprising a PBS |
| sequence | comprising DNA | and a SOI |
| RNA guide | Cas nuclease binding region | DNA template sequence comprising a PBS |
| sequence | comprising DNA | and a SOI |
| RNA guide | Cas nickase binding region | DNA template sequence comprising a PBS |
| sequence | comprising DNA | and a SOI |
| RNA guide | Cas binding region | DNA template sequence comprising a |
| sequence | comprising RNA | modified nucleotide |
| RNA guide | Cas nuclease binding region | DNA template sequence comprising a |
| sequence | comprising RNA | modified nucleotide |
| RNA guide | Cas nickase binding region | DNA template sequence comprising a |
| sequence | comprising RNA | modified nucleotide |
| RNA guide | Cas binding region | DNA template sequence comprising a |
| sequence | comprising DNA | modified nucleotide |
| RNA guide | Cas nuclease binding region | DNA template sequence comprising a |
| sequence | comprising DNA | modified nucleotide |
| RNA guide | Cas nickase binding region | DNA template sequence comprising a |
| sequence | comprising DNA | modified nucleotide |
| RNA guide | Cas binding region | DNA template sequence comprising a DNA |
| sequence | comprising RNA | polymerase recruitment moiety |
| RNA guide | Cas nuclease binding region | DNA template sequence comprising a DNA |
| sequence | comprising RNA | polymerase recruitment moiety |
| RNA guide | Cas nickase binding region | DNA template sequence comprising a DNA |
| sequence | comprising RNA | polymerase recruitment moiety |
| RNA guide | Cas binding region | DNA template sequence comprising a DNA |
| sequence | comprising DNA | polymerase recruitment moiety |
| RNA guide | Cas nuclease binding region | DNA template sequence comprising a DNA |
| sequence | comprising DNA | polymerase recruitment moiety |
| RNA guide | Cas nickase binding region | DNA template sequence comprising a DNA |
| sequence | comprising DNA | polymerase recruitment moiety |
| RNA guide | Cas binding region | DNA template sequence comprising a DNA |
| sequence | comprising RNA | ligase recruitment moiety |
| RNA guide | Cas nuclease binding region | DNA template sequence comprising a DNA |
| sequence | comprising RNA | ligase recruitment moiety |
| RNA guide | Cas nickase binding region | DNA template sequence comprising a DNA |
| sequence | comprising RNA | ligase recruitment moiety |
| RNA guide | Cas binding region | DNA template sequence comprising a DNA |
| sequence | comprising DNA | ligase recruitment moiety |
| RNA guide | Cas nuclease binding region | DNA template sequence comprising a DNA |
| sequence | comprising DNA | ligase recruitment moiety |
| RNA guide | Cas nickase binding region | DNA template sequence comprising a DNA |
| sequence | comprising DNA | ligase recruitment moiety |
| RNA/DNA guide | Cas binding region | DNA template sequence |
| sequence | comprising RNA | |
| RNA/DNA guide | Cas nuclease binding region | DNA template sequence |
| sequence | comprising RNA | |
| RNA/DNA guide | Cas nickase binding region | DNA template sequence |
| sequence | comprising RNA | |
| RNA/DNA guide | Cas binding region | DNA template sequence |
| sequence | comprising DNA | |
| RNA/DNA guide | Cas nuclease binding region | DNA template sequence |
| sequence | comprising DNA | |
| RNA/DNA guide | Cas nickase binding region | DNA template sequence |
| sequence | comprising DNA | |
| RNA/DNA guide | Cas binding region | DNA template sequence comprising a PBS |
| sequence | comprising RNA | |
| RNA/DNA guide | Cas nuclease binding region | DNA template sequence comprising a PBS |
| sequence | comprising RNA | |
| RNA/DNA guide | Cas nickase binding region | DNA template sequence comprising a PBS |
| sequence | comprising RNA | |
| RNA/DNA guide | Cas binding region | DNA template sequence comprising a PBS |
| sequence | comprising DNA | |
| RNA/DNA guide | Cas nuclease binding region | DNA template sequence comprising a PBS |
| sequence | comprising DNA | |
| RNA/DNA guide | Cas nickase binding region | DNA template sequence comprising a PBS |
| sequence | comprising DNA | |
| RNA/DNA guide | Cas binding region | DNA template sequence comprising a SOI |
| sequence | comprising RNA | |
| RNA/DNA guide | Cas nuclease binding region | DNA template sequence comprising a SOI |
| sequence | comprising RNA | |
| RNA/DNA guide | Cas nickase binding region | DNA template sequence comprising a SOI |
| sequence | comprising RNA | |
| RNA/DNA guide | Cas binding region | DNA template sequence comprising a SOI |
| sequence | comprising DNA | |
| RNA/DNA guide | Cas nuclease binding region | DNA template sequence comprising a SOI |
| sequence | comprising DNA | |
| RNA/DNA guide | Cas nickase binding region | DNA template sequence comprising a SOI |
| sequence | comprising DNA | |
| RNA/DNA guide | Cas binding region | DNA template sequence comprising a PBS |
| sequence | comprising RNA | and a SOI |
| RNA/DNA guide | Cas nuclease binding region | DNA template sequence comprising a PBS |
| sequence | comprising RNA | and a SOI |
| RNA/DNA guide | Cas nickase binding region | DNA template sequence comprising a PBS |
| sequence | comprising RNA | and a SOI |
| RNA/DNA guide | Cas binding region | DNA template sequence comprising a PBS |
| sequence | comprising DNA | and a SOI |
| RNA/DNA guide | Cas nuclease binding region | DNA template sequence comprising a PBS |
| sequence | comprising DNA | and a SOI |
| RNA/DNA guide | Cas nickase binding region | DNA template sequence comprising a PBS |
| sequence | comprising DNA | and a SOI |
| RNA/DNA guide | Cas binding region | DNA template sequence comprising a |
| sequence | comprising RNA | modified nucleotide |
| RNA/DNA guide | Cas nuclease binding region | DNA template sequence comprising a |
| sequence | comprising RNA | modified nucleotide |
| RNA/DNA guide | Cas nickase binding region | DNA template sequence comprising a |
| sequence | comprising RNA | modified nucleotide |
| RNA/DNA guide | Cas binding region | DNA template sequence comprising a |
| sequence | comprising DNA | modified nucleotide |
| RNA/DNA guide | Cas nuclease binding region | DNA template sequence comprising a |
| sequence | comprising DNA | modified nucleotide |
| RNA/DNA guide | Cas nickase binding region | DNA template sequence comprising a |
| sequence | comprising DNA | modified nucleotide |
| RNA/DNA guide | Cas binding region | DNA template sequence comprising a DNA |
| sequence | comprising RNA | polymerase recruitment moiety |
| RNA/DNA guide | Cas nuclease binding region | DNA template sequence comprising a DNA |
| sequence | comprising RNA | polymerase recruitment moiety |
| RNA/DNA guide | Cas nickase binding region | DNA template sequence comprising a DNA |
| sequence | comprising RNA | polymerase recruitment moiety |
| RNA/DNA guide | Cas binding region | DNA template sequence comprising a DNA |
| sequence | comprising DNA | polymerase recruitment moiety |
| RNA/DNA guide | Cas nuclease binding region | DNA template sequence comprising a DNA |
| sequence | comprising DNA | polymerase recruitment moiety |
| RNA/DNA guide | Cas nickase binding region | DNA template sequence comprising a DNA |
| sequence | comprising DNA | polymerase recruitment moiety |
| RNA/DNA guide | Cas binding region | DNA template sequence comprising a DNA |
| sequence | comprising RNA | ligase recruitment moiety |
| RNA/DNA guide | Cas nuclease binding region | DNA template sequence comprising a DNA |
| sequence | comprising RNA | ligase recruitment moiety |
| RNA/DNA guide | Cas nickase binding region | DNA template sequence comprising a DNA |
| sequence | comprising RNA | ligase recruitment moiety |
| RNA/DNA guide | Cas binding region | DNA template sequence comprising a DNA |
| sequence | comprising DNA | ligase recruitment moiety |
| RNA/DNA guide | Cas nuclease binding region | DNA template sequence comprising a DNA |
| sequence | comprising DNA | ligase recruitment moiety |
| RNA/DNA guide | Cas nickase binding region | DNA template sequence comprising a DNA |
| sequence | comprising DNA | ligase recruitment moiety |

In some embodiments, any of the Cas nuclease binding regions bind to a Cas9 or a Cas12a nuclease. In some embodiments, any of the Cas nickase binding regions bind to a Cas9 nickase or a Cas12a nickase. In some embodiments, the polynucleotide comprises a spacer between any of the guide sequence (e.g., RNA guide sequence or RNA/DNA guide sequence), the Cas binding region (e.g., Cas nuclease binding region or Cas nickase binding region), and the DNA template sequence described above.

The disclosure herein envisions that the compositions described herein can comprise more than one polynucleotide, e.g., two polynucleotides. For exemplary purposes only, the compositions can comprise two polynucleotides can include the features as shown in Table or Table 3 (from 5′ to 3′):

TABLE 2

| First polynucleotide | | | Second polynucleotide | |
|---|---|---|---|---|
| RNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | RNA | RNA | | moiety, DNA ligase |
| | | | | recruitment moiety, or |
| | | | | combinations thereof |
| RNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | RNA | | | recruitment moiety, or |
| | | | | combinations thereof |
| RNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | RNA | | | recruitment moiety, or |
| | | | | combinations thereof |
| RNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | DNA | RNA | | moiety, DNA ligase |
| | | | | recruitment moiety, or |
| | | | | combinations thereof |
| RNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | DNA | | | recruitment moiety, or |
| | | | | combinations thereof |
| RNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | DNA | | | recruitment moiety, or |
| | | | | combinations thereof |
| RNA/DNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | RNA | RNA | | moiety, DNA ligase |
| | | | | recruitment moiety, or |
| | | | | combinations thereof |
| RNA/DNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | RNA | | | recruitment moiety, or |
| | | | | combinations thereof |
| RNA/DNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | RNA | | | recruitment moiety, or |
| | | | | combinations thereof |
| RNA/DNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | DNA | RNA | | moiety, DNA ligase |
| | | | | recruitment moiety, or |
| | | | | combinations thereof |
| RNA/DNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | DNA | | | recruitment moiety, or |
| | | | | combinations thereof |
| RNA/DNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | RNA | | moiety, DNA ligase |
| | DNA | | | recruitment moiety, or |
| | | | | combinations thereof |
| RNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | RNA | DNA | | moiety, DNA ligase |
| | | | | recruitment moiety, or |
| | | | | combinations thereof |
| RNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | RNA | | | recruitment moiety, or |
| | | | | combinations thereof |
| RNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | RNA | | | recruitment moiety, or |
| | | | | combinations thereof |
| RNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | DNA | DNA | | moiety, DNA ligase |
| | | | | recruitment moiety, or |
| | | | | combinations thereof |
| RNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | DNA | | | recruitment moiety, or |
| | | | | combinations thereof |
| RNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | DNA | | | recruitment moiety, or |
| | | | | combinations thereof |
| RNA/DNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | RNA | DNA | | moiety, DNA ligase |
| | | | | recruitment moiety, or |
| | | | | combinations thereof |
| RNA/DNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | RNA | | | recruitment moiety, or |
| | | | | combinations thereof |
| RNA/DNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | RNA | | | recruitment moiety, or |
| | | | | combinations thereof |
| RNA/DNA | Cas | First | Second hybridization | DNA template region |
| guide | binding | hybridization | region comprising | comprising a PBS, a SOI, a |
| sequence | region | region | RNA or DNA | modified nucleotide, DNA |
| | comprising | comprising | | polymerase recruitment |
| | DNA | DNA | | moiety, DNA ligase |
| | | | | recruitment moiety, or |
| | | | | combinations thereof |
| RNA/DNA | Cas | First | Second hybridization | DNA template region |
| guide | nuclease | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | DNA | | | recruitment moiety, or |
| | | | | combinations thereof |
| RNA/DNA | Cas | First | Second hybridization | DNA template region |
| guide | nickase | hybridization | region comprising | comprising a PBS, a SOI, a |
| sequence | binding | region | RNA or DNA | modified nucleotide, DNA |
| | region | comprising | | polymerase recruitment |
| | comprising | DNA | | moiety, DNA ligase |
| | DNA | | | recruitment moiety, or |
| | | | | combinations thereof |

TABLE 3

| First polynucleotide | | Second polynucleotide | | |
|---|---|---|---|---|
| RNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | RNA | RNA or DNA | RNA | recruitment moiety, or |
| | | | | combinations thereof |
| RNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | RNA | combinations thereof |
| RNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | RNA | combinations thereof |
| RNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | RNA | RNA or DNA | DNA | recruitment moiety, or |
| | | | | combinations thereof |
| RNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | DNA | combinations thereof |
| RNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | DNA | combinations thereof |
| RNA/DNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | RNA | RNA or DNA | RNA | recruitment moiety, or |
| | | | | combinations thereof |
| RNA/DNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | RNA | combinations thereof |
| RNA/DNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | RNA | combinations thereof |
| RNA/DNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | RNA | RNA or DNA | DNA | recruitment moiety, or |
| | | | | combinations thereof |
| RNA/DNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | DNA | combinations thereof |
| RNA/DNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | RNA | RNA or DNA | comprising | recruitment moiety, or |
| | | | DNA | combinations thereof |
| RNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | DNA | RNA or DNA | RNA | recruitment moiety, or |
| | | | | combinations thereof |
| RNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | RNA | combinations thereof |
| RNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | RNA | combinations thereof |
| RNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | DNA | RNA or DNA | DNA | recruitment moiety, or |
| | | | | combinations thereof |
| RNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | DNA | combinations thereof |
| RNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | DNA | combinations thereof |
| RNA/DNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | DNA | RNA or DNA | RNA | recruitment moiety, or |
| | | | | combinations thereof |
| RNA/DNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | RNA | combinations thereof |
| RNA/DNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | RNA | combinations thereof |
| RNA/DNA | First | Second | Cas | DNA template region comprising |
| guide | hybridization | hybridization | binding | a PBS, a SOI, a modified |
| sequence | region | region | region | nucleotide, DNA polymerase |
| | comprising | comprising | comprising | recruitment moiety, DNA ligase |
| | DNA | RNA or DNA | DNA | recruitment moiety, or |
| | | | | combinations thereof |
| RNA/DNA | First | Second | Cas | DNA template region comprising |
| guide | hybridization | hybridization | nuclease | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | DNA | combinations thereof |
| RNA/DNA | First | Second | Cas | DNA template region comprising |
| guide | hybridization | hybridization | nickase | a PBS, a SOI, a modified |
| sequence | region | region | binding | nucleotide, DNA polymerase |
| | comprising | comprising | region | recruitment moiety, DNA ligase |
| | DNA | RNA or DNA | comprising | recruitment moiety, or |
| | | | DNA | combinations thereof |

In some embodiments, the DNA template sequence of any of the second polynucleotides described above comprises a primer binding sequence and sequence of interest. In some embodiments, the DNA template sequence of any of the second polynucleotides described above comprises a primer binding sequence, sequence of interest, and one or more of a modified nucleotide, DNA polymerase recruitment moiety, DNA ligase recruitment moiety.

In some embodiments, any of the Cas nuclease binding regions bind to a Cas9 or a Cas12a nuclease. In some embodiments, any of the Cas nickase binding regions bind to a Cas9 nickase or a Cas12a nickase. In some embodiments, the first and/or second polynucleotide comprises a spacer between any of the guide sequence (e.g., RNA guide sequence or RNA/DNA guide sequence), the Cas binding region (e.g., Cas nuclease binding region or Cas nickase binding region), and the DNA template sequence described above.

### Fusion Protein

In some embodiments, composition of the present disclosure comprises a Cas nuclease or Cas nickase, wherein the Cas nuclease or Cas nickase is fused to a DNA polymerase recruitment protein. In some embodiments, the disclosure provides a fusion protein comprising (i) a Cas nuclease or a Cas nickase; and (ii) a DNA polymerase recruitment protein. In some embodiments, the disclosure provides a fusion protein comprising (i) a Cas nuclease or a Cas nickase; and (ii) a DNA polymerase recruitment protein, a DNA ligase, a DNA ligase recruitment moiety, a DNA binding protein, a DNA repair protein, or combination thereof.

As discussed herein, a fusion protein typically includes at least two domains having different functions. In some embodiments, the fusion protein comprises a Cas nuclease. Cas nucleases are described herein. In some embodiments, the Cas nuclease is a Cas9 nuclease. In some embodiments, the Cas nuclease is a Cas12a nuclease. In some embodiments, the Cas nuclease is a Type II-B Cas nuclease. In some embodiments, the fusion protein comprises a Cas nickase. Cas nickases are further described herein. In some embodiments, the Cas nickase is a Cas9 nickase. In some embodiments, the Cas nickase is a Cas12a nickase. In some embodiments, the Cas nickase is a Type II-B Cas nickase.

In some embodiments, fusion protein comprises a DNA polymerase recruitment protein. DNA polymerase recruitment proteins are further described herein. In some embodiments, the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

In some embodiments, the DNA polymerase recruitment protein is capable of recruiting a DNA polymerase, wherein the DNA polymerase comprises Pol I or a Klenow fragment thereof, Pol II, Pol III, Pol IV, Pol V, Pol α, Pol β, Pol λ, Pol γ, Pol σ, Pol μ, Pol δ, Pol ε, Pol η, Pol ι, Pol κ, Pol ζ, Pol θ, REV1, REV3, Bst DNA polymerase, T4 DNA polymerase, Φ29 (phi29) DNA polymerase, Taq DNA polymerase, Pfu DNA polymerase, KOD DNA polymerase, Tth DNA polymerase, Pwo DNA polymerase, or a variant or homologue thereof.

In some embodiments, the DNA ligase comprises T4 DNA ligase, Paramecium bursaria Chlorella virus 1 (PCBV-1) DNA ligase, Mycobacterium Ligase D (LigD), human ligase 1 (Lig1), human ligase 3 (Lig3a), human ligase 4 (Lig4), or combination thereof. In some embodiments, the DNA ligase recruitment moiety comprises a 5′-adenylpyrophosphoryl cap.

In some embodiments, the DNA binding protein comprises replication protein A (RPA) or a subunit thereof, including RPA1, RPA2, and RPA3, a Single-Stranded DNA Binding Protein (SSBP), including SSBP1, SSBP2, SSBP3, and SSBP4, or combination hereof. In some embodiments, the DNA repair protein comprises Tyrosyl-DNA Phosphodiesterase 1 (TDP1), aprataxin, topoisomerase I, or combination thereof.

In some embodiments, one or more polynucleotides comprising a guide sequence, Cas-binding region, and DNA template sequence as described herein is provided to the fusion protein. In some embodiments, the Cas nuclease or Cas nickase of the fusion protein is capable of binding to and cleave a target polynucleotide. In some embodiments, the DNA polymerase recruitment moiety of the fusion protein recruits a DNA polymerase to the cleaved target polynucleotide. In some embodiments, the recruited DNA polymerase synthesizes a DNA strand complementary to a sequence of interest on the DNA template sequence, thereby producing a double-stranded sequence comprising the sequence of interest that can be inserted into the cleavage target polynucleotide.

In some embodiments, the fusion protein increases efficiency of inserting the double-stranded sequence into the cleaved target polynucleotide by recruiting a DNA polymerase, a DNA ligase, and/or a DNA repair protein, thereby increasing the efficiency of producing the double-stranded sequence. In some embodiments, the fusion protein comprising the DNA polymerase recruitment moiety, DNA ligase, DNA ligase recruitment moiety, DNA binding protein, DNA repair protein, or combination thereof, has higher insertion efficiency as compared to a Cas nuclease or Cas nickase that is not fused to a DNA polymerase recruitment moiety, DNA ligase, DNA ligase recruitment moiety, DNA binding protein, DNA repair protein, or combination thereof, as described herein.

In some embodiments, the fusion protein further comprises a nuclear localization signal (NLS). As used herein, “nuclear localization signal” or “nuclear localization sequence” (NLS) refers to a polypeptide that “tags” a protein for import into the cell nucleus by nuclear transport, i.e., a protein having a NLS is transported into the cell nucleus. Typically, the NLS includes positively-charged Lys or Arg residues exposed on the protein surface. Exemplary NLS's include, but are not limited to, the NLS from: SV40 Large T-Antigen, nucleoplasmin, EGL-13, c-Myc, and TUS-protein.

In some embodiments, the fusion protein further comprises a linker that links the Cas nuclease or Cas nickase and the DNA polymerase recruitment protein. In some embodiments, the linker is of sufficient length and/or flexibility such that the Cas nuclease or Cas nickase can be positioned without steric hindrance from the DNA polymerase recruitment protein. In some embodiments, the linker comprises about 3 to about 100 amino acids in length. In some embodiments, the linker comprises about 5 to about 80 amino acids in length. In some embodiments, the linker comprises about 10 to about 60 amino acids in length. In some embodiments, the linker comprises about 20 to about 50 amino acid sin length. In some embodiments, the linker comprises about 25 to about 40 amino acids in length.

In some embodiments, the disclosure provides a polynucleotide encoding the fusion protein described herein. In some embodiments, the polynucleotide is a codon-optimized polynucleotide for expression in a bacterial cell. In some embodiments, the polynucleotide is a codon-optimized polynucleotide for expression in a eukaryotic cell. In some embodiments, the polynucleotide is a codon-optimized polynucleotide for expression in a mammalian cell. In some embodiments, the polynucleotide is a codon-optimized polynucleotide for expression in a human cell. As used herein, “codon optimization” refers to the adjustment of codons to match the expression host's tRNA abundance in order to increase yield and efficiency of recombinant or heterologous protein expression. Codon optimization methods are known in the art and may be performed using software programs such as, for example, the Codon Optimization tool from Integrated DNA Technologies, the Codon Usage Table analysis tool from Entelechon, and the like.

In some embodiments, the disclosure provides a vector comprising the polynucleotide that encodes the fusion protein described herein. Various types of vectors, e.g., viral and non-viral vectors, are provided herein. In some embodiments, the vector is an expression vector. In some embodiments, the vector is a bacterial expression vector. In some embodiments, the vector is a mammalian expression vector. In some embodiments, the vector is a human expression vector. In some embodiments, the vector is a plant expression vector.

In some embodiments, the vector is a viral vector. In some embodiments, the viral vector is a retrovirus, adeno-associated virus, pox, baculovirus, vaccinia, herpes simplex, Epstein-Barr virus, adenovirus, geminivirus, or caulimovirus vector. In some embodiments, the viral vector is an adenovirus, a lentivirus, or an adeno-associated viral vector. Viral transduction with adenovirus, adeno-associated virus (AAV), and lentiviral vectors (wherein administration can be local, targeted or systemic) have been used as delivery methods for in vivo gene therapy. Methods of introducing vectors, e.g., viral vectors, into cells (e.g., transfection) are described herein.

In some embodiments, the vector further comprises a regulatory element operably linked to the polynucleotide encoding the fusion protein. In some embodiments, the regulatory element comprises a promoter, an enhancer, a terminator, a 5′ UTR, a 3′ UTR, or combination thereof. Regulatory elements are further described herein. In some embodiments, the regulatory element comprises a promoter. In some embodiments, the promoter is a bacterial promoter. In some embodiments, the promoter is a viral promoter. In some embodiments, the promoter is a mammalian promoter.

In some embodiments, the disclosure provides a kit comprising the fusion protein described herein. In some embodiments, the fusion protein in the kit is provided as a polynucleotide encoding the fusion protein. In some embodiments, the polynucleotide encoding the fusion protein is provided on a vector, e.g., a vector described herein.

In some embodiments, the kit further comprises a polynucleotide, wherein the polynucleotide comprises a Cas-binding region. In some embodiments, the Cas-binding region is capable of binding to the Cas nuclease or Cas nickase of the fusion protein. In some embodiments, the Cas-binding region comprises a tracrRNA. In some embodiments, the Cas-binding region is capable of hybridizing with a tracrRNA. In some embodiments, the kit further comprises a tracrRNA. Polynucleotides comprising Cas-binding regions are further described herein.

In some embodiments, the kit further comprises a DNA polymerase. In some embodiments, the DNA polymerase comprises phi29 DNA polymerase, DNA polymerase mu, DNA polymerase delta, or DNA polymerase epsilon. In some embodiments, the kit further comprises a DNA ligase. In some embodiments, the DNA ligase comprises T4 DNA ligase, PCBV-1 DNA ligase, LigD, human Lig1, human Lig3a, or human Lig4.

In some embodiments, the kit further comprises a reaction buffer and/or a storage buffer for the fusion protein, the DNA polymerase, and/or the DNA ligase. In some embodiments, the kit further comprises a reagent for performing a DNA cleavage reaction, a DNA polymerase reaction, and/or a DNA ligase reaction. In some embodiments, the reagent comprises ATP, dNTPs, MgCl₂, Oligo(dT), and/or an RNase inhibitor. In some embodiments, the kit comprises one or more controls, e.g., a control target DNA. For example, the control target DNA can be designed to be cleaved specifically by the Cas nuclease or Cas nickase of the fusion protein with a certain amount of efficiency, thereby calibrating the activity of the Cas nuclease or Cas nickase.

### Cells

In some embodiments, the disclosure provides a cell comprising the fusion protein described herein. In some embodiments, the disclosure provides a cell comprising the polynucleotide that encodes the fusion protein described herein. In some embodiments, the disclosure provides a cell comprising the vector that comprises the polynucleotide encoding the fusion protein described herein. In some embodiments, the cell further comprises a polynucleotide described herein, wherein the polynucleotide comprises a guide sequence (e.g., RNA guide sequence), Cas-binding protein, and DNA template sequence.

In some embodiments, the disclosure provides a cell comprising a polynucleotide described herein, wherein the polynucleotide comprises an RNA guide sequence, a Cas-binding region, and DNA template sequence. In some embodiments, the disclosure provides a cell comprising a composition described herein, wherein the composition comprises: a Cas nuclease or Cas nickase; and one or more polynucleotides comprising a guide sequence, a Cas-binding region, and a DNA template sequence. In some embodiments, the disclosure provides a cell comprising a composition described herein, wherein the composition comprises (a) a Cas nuclease or Cas nickase and (b) a polynucleotide comprising a guide sequence, a Cas-binding region, and a DNA template sequence, wherein the DNA template sequence is a 3′ end of the polynucleotide. In some embodiments, the disclosure provides a cell comprising a composition described herein, wherein the composition comprises (a) a Cas nuclease or a Cas nickase; (b) a first polynucleotide comprising: (i) a guide sequence; (ii) a Cas-binding region; and (iii) a first hybridization region, wherein the first hybridization region is at a 3′ end of the first polynucleotide; and (c) a second polynucleotide comprising: (i) a second hybridization region that is complementary to the first hybridization region and (ii) a DNA template sequence. In some embodiments, the disclosure provides a cell comprising a composition described herein, wherein the composition comprises (a) a Cas nuclease or a Cas nickase; (b) a first polynucleotide comprising: (i) a guide sequence; and (ii) a first hybridization region, wherein the first hybridization region is at a 3′ end of the first polynucleotide; and (c) a second polynucleotide comprising: (i) a second hybridization region that is complementary to the first hybridization region; (ii) a Cas-binding region; and (iii) a DNA template sequence. Components of the polynucleotide and the composition are further described herein. In some embodiments, the Cas nuclease or Cas nickase is fused to a DNA polymerase recruitment protein. Fusion proteins comprising a Cas nuclease or Cas nickase and a DNA polymerase recruitment protein are further described herein.

In some embodiments, the disclosure provides a cell comprising a composition described herein, wherein the composition comprises: (a) a Cas nuclease or a Cas nickase; (b) a first polynucleotide comprising: (i) a guide sequence; (ii) a Cas-binding region; (iii) a first hybridization region; and (iv) a primer binding sequence, wherein the primer binding sequence is at a 3′ end of the first polynucleotide; and (c) a second polynucleotide comprising: (i) a second hybridization region that is complementary to the first hybridization region; and (ii) a sequence of interest (SOI). In some embodiments, the disclosure provides a cell comprising a composition described herein, wherein the composition comprises: (a) a Cas nuclease or a Cas nickase; (b) a first polynucleotide comprising (i) a guide sequence; (ii) a Cas-binding region; and (iii) a first hybridization region, wherein the first hybridization region is at a 3′ end of the first polynucleotide; (c) a second polynucleotide comprising: (i) a second hybridization region that is complementary to the first hybridization region; (ii) a third hybridization region; and (iii) a primer binding sequence, wherein the primer binding sequence is at a 3′ end of the second polynucleotide; and (d) a third polynucleotide comprising: (i) a fourth hybridization region that is complementary to the third hybridization region; and (ii) a sequence of interest (SOI). Components of the compositions are further described herein. In some embodiments, the Cas nuclease or Cas nickase is fused to a DNA polymerase recruitment moiety, a DNA ligase, a DNA ligase recruitment moiety, a DNA binding protein, a DNA repair protein, or combination thereof. Fusion proteins are further described herein.

In some embodiments, the cell comprises an endogenous DNA polymerase, an endogenous DNA ligase, or both. In some embodiments, the cell does not comprise an exogenous DNA polymerase. In some embodiments, the cell does not comprise an exogenous DNA ligase.

In some embodiments, the cell further comprises an exogenous DNA polymerase, an exogenous DNA ligase, or both. In some embodiments, the exogenous DNA polymerase is homologous to the cell. In some embodiments, the exogenous DNA polymerase is heterologous to the cell. In some embodiments, the exogenous DNA polymerase is derived from a different organism than the cell. For example, the cell may be an E. coli cell, and the DNA polymerase may be a T4 DNA polymerase. In some embodiments, the exogenous DNA polymerase comprises Pol I or a Klenow fragment thereof, Pol II, Pol III, Pol IV, Pol V, Pol α, Pol β, Pol λ, Pol γ, Pol σ, Pol μ, Pol δ, Pol ε, Pol η, Pol ι, Pol κ, Pol ζ, Pol θ, REV1, REV3, Bst DNA polymerase, T4 DNA polymerase, Φ29 (phi29) DNA polymerase, Taq DNA polymerase, Pfu DNA polymerase, KOD DNA polymerase, Tth DNA polymerase, Pwo DNA polymerase, or a variant or homologue thereof. In some embodiments, the exogenous DNA ligase is homologous to the cell. In some embodiments, the exogenous DNA ligase is heterologous to the cell. In some embodiments, the exogenous DNA ligase is derived from a different organism than the cell. In some embodiments, the exogenous DNA ligase comprises E. coli DNA ligase, T4 DNA ligase, T7 DNA ligase, DNA ligase I, DNA ligase II, DNA ligase III, DNA ligase IV, Taq DNA ligase, or a variant or homologue thereof. For example, the cell may be an E. coli cell, and the DNA ligase may be a T4 ligase.

In some embodiments, the cell is a bacterial cell. In some embodiments, the bacterial cell is a laboratory strain. Examples of such bacterial cells include, but are not limited to, E. coli, S. aureus, V. cholerae, S. pneumoniae, B. subtilis, C. crescentus, M genitalium, A. fischeri, Synechocystis, P. fluorescens, A. vinelandii, S. coelicolor. In some embodiments, the bacterial cell is of bacteria used in preparation of food and/or beverages. Non-limiting exemplary genera of such cells include, but are not limited to, Acetobacter, Arthrobacter, Bacillus, Bifidobacterium, Brachybacterium, Brevibacterium, Carnobacterium, Corynebacterium, Enterococcus, Gluconacetobacter, Hafnia, Halomonas, Kocuria, Lactobacillus (including L. acetotolerans, L. acidipiscis, L. acidophilus, L. alimentarius, L. brevis, L. bucheri, L. casei, L. curvatus, L. fermentum, L. hilgardii, L. jensenii, L. kimchii, L. lactis, L. paracasei, L. plantarum, and L. sakei), Leuconostoc, Microbacterium, Pediococcus, Propionibacterium, Weissella, and Zymomonas.

In some embodiments, the cell is a eukaryotic cell. In some embodiments, the eukaryotic cell is a mammalian cell. In some embodiments, the eukaryotic cell is an animal cell. In some embodiments, the eukaryotic cell is a mammalian cell. In some embodiments, the eukaryotic cell is of an animal or human cell, cell line, or cell strain. Examples of animal or mammalian cells, cell lines, or cell strains include, but are not limited to, mouse myeloma (NSO), Chinese hamster ovary (CHO), HT1080, H9, HepG2, MCF7, MDBK Jurkat, NIH3T3, PC12, BHK (baby hamster kidney), EBX, EB14, EB24, EB26, EB66, or Ebv13, VERO, SP2/0, YB2/0, Y0, C127, L cell, COS (e.g., COS1 and COS7), QC1-3, HEK293, VERO, PER.C6, HeLA, EB1, EB2, EB3, oncolytic cell, or hybridoma cell. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) can be, for example, a CHO-K1 SV GS knockout cell.

In some embodiments, the eukaryotic cell is a human stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57Bl/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potato, tomato, eggplant, pepper, paprika; plants of the genus Brassica, plants of the genus Lactuca; plants of the genus Spinacia; plants of the genus Capsicum; cotton, tobacco, asparagus, carrot, cabbage, broccoli, cauliflower, lettuce, spinach, strawberry, blueberry, raspberry, blackberry, grape, coffee, cocoa, and the like.

### Methods

In some embodiments, the disclosure provides a method of providing a targeted insertion in a target polynucleotide in a cell, comprising introducing a composition of the present disclosure into the cell. In some embodiments, the target polynucleotide is a target DNA. In some embodiments, the composition comprises: a Cas nuclease or Cas nickase; and one or more polynucleotides comprising a guide sequence, a Cas-binding region, and a DNA template sequence.

In some embodiments, the composition comprises (a) a Cas nuclease or Cas nickase and (b) a polynucleotide comprising a guide sequence, a Cas-binding region, and a DNA template sequence, wherein the DNA template sequence is a 3′ end of the polynucleotide. In some embodiments, the composition comprises (a) a Cas nuclease or a Cas nickase; (b) a first polynucleotide comprising: (i) a guide sequence; (ii) a Cas-binding region; and (iii) a first hybridization region, wherein the first hybridization region is at a 3′ end of the first polynucleotide; and (c) a second polynucleotide comprising: (i) a second hybridization region that is complementary to the first hybridization region and (ii) a DNA template sequence. In some embodiments, the composition comprises (a) a Cas nuclease or a Cas nickase; (b) a first polynucleotide comprising: (i) a guide sequence; and (ii) a first hybridization region, wherein the first hybridization region is at a 3′ end of the first polynucleotide; and (c) a second polynucleotide comprising: (i) a second hybridization region that is complementary to the first hybridization region; (ii) a Cas-binding region; and (iii) a DNA template sequence. Components of the polynucleotide and the composition are further described herein.

An exemplary, non-limiting outline of the method is illustrated in FIG. 1. In FIG. 1, a Cas nuclease is guided to a target DNA in a cell by a guide sequence and cleaves the target DNA. The DNA template sequence comprises (i) a primer binding sequence that hybridizes with the cleaved DNA, which serves as a primer, and (ii) a sequence of interest. A DNA polymerase, e.g., endogenous DNA polymerase of the cell, binds to the primer and synthesizes a DNA strand complementary to the sequence of interest, thereby forming a double-stranded sequence comprising the sequence of interest. The double-stranded sequence can be inserted into the cleaved target DNA by a DNA repair pathway, e.g., NHEJ.

In some embodiments, the method of providing a targeted insertion in a target DNA in a cell comprises: introducing the composition described herein into the cell, wherein the guide sequence is capable of hybridizing to the target DNA. In some embodiments, the DNA template sequence comprises single-stranded DNA. In some embodiments, the guide sequence is capable of hybridizing to the target DNA. In some embodiments, the Cas nuclease or Cas nickase is guided to the target DNA via hybridization of the guide sequence and the target DNA. In some embodiments, the method is performed under conditions sufficient for the Cas nuclease or Cas nickase to generate a cleavage in the target DNA.

In some embodiments, one strand of the cleaved target DNA is a primer for a DNA polymerase. In some embodiments, the DNA template sequence comprises a primer binding sequence and a sequence of interest. In some embodiments, the primer binding sequence is capable of binding to the primer. In some embodiments, the method does not comprise introducing an exogenous DNA polymerase into the cell. In some embodiments, the method is performed under conditions sufficient for an endogenous DNA polymerase of the cell to bind the primer and extend the DNA template sequence. In some embodiments, the extending comprises synthesizing a DNA strand complementary to the sequence of interest to form a double-stranded sequence comprising the sequence of interest.

In some embodiments, the method comprises introducing an exogenous DNA polymerase into the cell. In some embodiments, the exogenous DNA polymerase is introduced into the cell via a polynucleotide encoding the exogenous DNA polymerase. In some embodiments, the exogenous DNA polymerase is introduced into the cell via vector comprising the polynucleotide. Methods of introducing exogenous components, e.g., an exogenous DNA polymerase, into a cell are described herein. In some embodiments, the exogenous DNA polymerase is homologous to the cell. In some embodiments, the exogenous DNA polymerase is heterologous to the cell. In some embodiments, the exogenous DNA polymerase is derived from a different organism than the cell. In some embodiments, the exogenous DNA polymerase comprises Pol I or a Klenow fragment thereof, Pol II, Pol III, Pol IV, Pol V, Pol α, Pol β, Pol λ, Pol γ, Pol σ, Pol μ, Pol δ, Pol ε, Pol η, Pol ι, Pol κ, Pol ζ, Pol θ, REV1, REV3, Bst DNA polymerase, T4 DNA polymerase, Φ29 (phi29) DNA polymerase, Taq DNA polymerase, Pfu DNA polymerase, KOD DNA polymerase, Tth DNA polymerase, Pwo DNA polymerase, or a variant or homologue thereof. In some embodiments, the method is performed under conditions sufficient for the exogenous DNA polymerase to bind the primer and extend the DNA template sequence. In some embodiments, the extending comprises synthesizing a DNA strand complementary to the sequence of interest to form a double-stranded sequence comprising the sequence of interest.

In some embodiments, the double-stranded sequence is inserted into the cleaved target DNA. In some embodiments, the double-stranded sequence comprising the sequence of interest is inserted into the cleaved target sequence by a DNA repair pathway. DNA repair pathways include the non-homologous end joining (NHEJ) pathway, microhomology-mediated end joining (MMEJ) pathway, the homology-directed repair (HDR) pathway, synthesis-dependent strand annealing (SDSA), single-stranded annealing (SSA), and alternative end joining (Alt-EJ). NHEJ does not require a homologous template. In general, NHEJ has higher repair efficiency but lower fidelity when compared with HDR, although errors decrease when the double-stranded breaks have compatible cohesive ends or overhangs. MMEJ, which has micro-homologies (e.g., of about 2 to about 10 base pairs) on both sides of a double-stranded break. HDR requires a homologous template to direct repair, and HDR repairs are typically high-fidelity but low efficiency compared with NHEJ and MMEJ. SDSA, SSA, and Alt-EJ are HDR-based repair pathways. SDSA is further described, e.g., in Sung et al., Nat Rev Mol Cell Biol 7:741(2006). SSA and Alt-EJ are further described in, e.g., Bhargava et al., Trends Genet 32(9):566-575 (2016). In some embodiments, the method is performed under conditions sufficient for non-homologous end joining (NHEJ). In some embodiments, the double-stranded sequence comprising the sequence of interest is inserted into the cleaved target sequence by NHEJ. In some embodiments, the method is performed under conditions sufficient for HDR, SDSA, SSA, Alt-EJ, or combination thereof.

In some embodiments, the double-stranded sequence is inserted into the cleaved target DNA by ligation. In some embodiments, the double-stranded sequence comprising the sequence of interest is inserted into the cleaved target sequence by a DNA ligase. In some embodiments, the DNA ligase is an endogenous DNA ligase of the cell. In some embodiments, the method does not comprise introducing an exogenous DNA ligase into the cell.

In some embodiments, the method further comprises introducing an exogenous DNA ligase into the cell, and the exogenous DNA ligase inserts the double-stranded sequence into the cleaved target DNA. In some embodiments, the exogenous DNA ligase is introduced into the cell via a polynucleotide encoding the exogenous DNA ligase. In some embodiments, the exogenous DNA ligase is introduced into the cell via vector comprising the polynucleotide. Methods of introducing exogenous components, e.g., an exogenous DNA ligase, into a cell are described herein. In some embodiments, the exogenous DNA ligase is homologous to the cell. In some embodiments, the exogenous DNA ligase is heterologous to the cell. In some embodiments, the exogenous DNA ligase is derived from a different organism than the cell. In some embodiments, the exogenous DNA ligase comprises E. coli DNA ligase, T4 DNA ligase, T7 DNA ligase, DNA ligase I, DNA ligase II, DNA ligase III, DNA ligase IV, Taq DNA ligase, or a variant or homologue thereof

In some embodiments, the double-stranded sequence further comprises a recognition site for an endonuclease, a transposase, or a recombinase. In some embodiments, the endonuclease, transposase, or recombinase integrates the double-stranded sequence into the target DNA. In some embodiments, the endonuclease, transposase, or recombinase is endogenous to the cell. Mechanisms of sequence integration by endonucleases, transposases, and recombinases are known to one of ordinary skill in the art and are further described, e.g., in Carlson et al., Mol Microbiol 27(4): 671-676 (1998), Nesmelova et al., Adv Drug Deliv Rev 62: 1187-1195 (2010), and Hallet et al., FEMS Microbiol Rev 21(2): 157-178 (1997).

In some embodiments, the DNA template sequence is on the same polynucleotide as the guide sequence and Cas-binding region and is therefore in proximity to the cleavage site on the target DNA. In some embodiments, the one or more polynucleotides comprising the DNA template sequence, the guide sequence, and the Cas-binding region are hybridized, and the DNA template sequence is therefore in proximity to the cleavage site on the target DNA. In some embodiments, proximity of the DNA template sequence to the cleavage site promotes insertion of the double-stranded sequence formed by the DNA polymerase into the cleaved target DNA.

In some embodiments, the present method increases efficiency of inserting the double-stranded sequence into the cleaved target DNA by providing the double-stranded sequence in proximity with the cleaved target DNA. In some embodiments, the present method increases efficiency of inserting the double-stranded sequence into the cleaved target DNA by reducing re-ligation of the cleaved target DNA. In some embodiments, the present method has improved efficiency compared with a method that that does not bring DNA template sequence in proximity to the cleaved target DNA. In some embodiments, the present method has at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, least 150-fold, or at least 200-fold or higher efficiency compared with a method that that does not bring a DNA template sequence in proximity to the cleaved target DNA.

In some embodiments, the disclosure provides a method of providing a targeted insertion in a target DNA, comprising contacting the composition described herein with the target DNA, wherein the guide sequence is capable of hybridizing to the target DNA. In some embodiments, the composition comprises: a Cas nuclease or Cas nickase; and one or more polynucleotides comprising a guide sequence, a Cas-binding region, a primer binding sequence, and a sequence of interest. In some embodiments, the composition comprises: (a) a Cas nuclease or a Cas nickase; (b) a first polynucleotide comprising: (i) a guide sequence; (ii) a Cas-binding region; (iii) a first hybridization region; and (iv) a primer binding sequence, wherein the primer binding sequence is at a 3′ end of the first polynucleotide; and (c) a second polynucleotide comprising: (i) a second hybridization region that is complementary to the first hybridization region; and (ii) a sequence of interest (SOI). In some embodiments, the composition comprises: (a) a Cas nuclease or a Cas nickase; (b) a first polynucleotide comprising (i) a guide sequence; (ii) a Cas-binding region; and (iii) a first hybridization region, wherein the first hybridization region is at a 3′ end of the first polynucleotide; (c) a second polynucleotide comprising: (i) a second hybridization region that is complementary to the first hybridization region; (ii) a third hybridization region; and (iii) a primer binding sequence, wherein the primer binding sequence is at a 3′ end of the second polynucleotide; and (d) a third polynucleotide comprising: (i) a fourth hybridization region that is complementary to the third hybridization region; and (ii) a sequence of interest (SOI). Components of the compositions are further described herein. In some embodiments, the Cas nuclease or Cas nickase is fused to a DNA polymerase recruitment moiety, a DNA ligase, a DNA ligase recruitment moiety, a DNA binding protein, a DNA repair protein, or combination thereof. Fusion proteins are further described herein.

In some embodiments, the Cas nuclease or Cas nickase is guided to the target DNA via hybridization of the guide sequence and the target DNA. In some embodiments, the method is performed under conditions sufficient for the Cas nuclease or Cas nickase to generate a cleavage in the target DNA. In some embodiments, one strand of the cleaved target DNA is a primer for a DNA polymerase. In some embodiments, the primer binding sequence of the first and/or the second polynucleotide is capable of binding to the primer.

In some embodiments, the method further comprises contacting the target DNA with a DNA ligase. DNA ligases are further described herein. In some embodiments, the DNA ligase is T4 ligase, PCBV-1 DNA ligase, LigD, a Human Ligase protein, or combination thereof. In some embodiments, the DNA ligase ligates the sequence of interest to the cleaved target DNA.

In some embodiments, the method further comprises contacting the target DNA with a DNA polymerase, a protein in a DNA repair pathway, or combination thereof. In some embodiments, the sequence of interest is a single-stranded sequence that is converted to a double-stranded sequence by the DNA polymerase, the protein in a DNA repair pathway, or combination thereof. DNA polymerases and DNA repair pathways are described herein.

In some embodiments, the method comprises contacting the target DNA with a cell extract, wherein the cell extract comprises the DNA ligase, DNA polymerase, and/or DNA repair pathway protein described herein. In some embodiments, the target DNA is contacted with a recombinant DNA ligase, DNA polymerase, and/or DNA repair pathway protein.

In some embodiments, the method is performed in vivo. In some embodiments, the method is performed in vitro. In some embodiments, the method is performed ex vivo. In some embodiments, the target DNA comprises a plasmid, a PCR product, a cosmid, a bacterial artificial chromosome (BAC), a yeast artificial chromosome (YAC), genomic DNA, mitochondrial DNA, chloroplast DNA, or combination thereof.

In some embodiments, the disclosure provides a method of providing a targeted insertion in a target polynucleotide in a cell, comprising introducing a composition of the present disclosure into the cell. In some embodiments, the target polynucleotide is a target DNA. In some embodiments, the composition comprises: a Cas nuclease or Cas nickase; and one or more polynucleotides comprising a guide sequence, a Cas-binding region, a primer binding sequence, and a sequence of interest. In some embodiments, the composition comprises: (a) a Cas nuclease or a Cas nickase; (b) a first polynucleotide comprising: (i) a guide sequence; (ii) a Cas-binding region; (iii) a first hybridization region; and (iv) a primer binding sequence, wherein the primer binding sequence is at a 3′ end of the first polynucleotide; and (c) a second polynucleotide comprising: (i) a second hybridization region that is complementary to the first hybridization region; (ii) a sequence of interest (SOI). In some embodiments, the composition comprises: (a) a Cas nuclease or a Cas nickase; (b) a first polynucleotide comprising (i) a guide sequence; (ii) a Cas-binding region; and (iii) a first hybridization region, wherein the first hybridization region is at a 3′ end of the first polynucleotide; (c) a second polynucleotide comprising: (i) a second hybridization region that is complementary to the first hybridization region; (ii) a third hybridization region; and (iii) a primer binding sequence, wherein the primer binding sequence is at a 3′ end of the second polynucleotide; and (d) a third polynucleotide comprising: (i) a fourth hybridization region that is complementary to the third hybridization region; (ii) a sequence of interest (SOI). Components of the compositions are further described herein. In some embodiments, the Cas nuclease or Cas nickase is fused to a DNA polymerase recruitment moiety, a DNA ligase, a DNA ligase recruitment moiety, a DNA binding protein, a DNA repair protein, or combination thereof. Fusion proteins are further described herein.

An exemplary, non-limiting outline of the method is illustrated in FIGS. 10A-10C. In FIG. 10A, the guide sequence, Cas-binding region, first hybridization region, and primer binding site are located on a first polynucleotide. A double-stranded break is generated at a target DNA by a Cas nuclease guided to the target DNA by a guide sequence. The primer binding site is hybridized to the cleaved DNA, and a second polynucleotide hybridizes to the first polynucleotide via the first and second hybridization regions that comprise complementary sequences. The second polynucleotide comprises a sequence of interest, which is ligated into the cleaved DNA at both the 5′ and 3′ ends by a ligase (FIG. 10A). The second polynucleotide can further include a homology sequence, and the 5′ end is ligated into the cleaved DNA by a ligase, and the 3′ end is integrated by a homology-mediated mechanism. In some embodiments, the homology-mediated mechanism comprises HDR, synthesis-dependent strand annealing (SDSA), single-stranded annealing (SSA), alternative end joining (alt-EJ), or combination thereof (FIG. 10B). Following the ligation and/or homology-mediated integration, the complex is converted into double-stranded DNA, thereby integrating the sequence of interest into the target DNA (FIG. 10C).

In some embodiments, the method of providing a targeted insertion in a target DNA in a cell comprises: introducing the composition described herein into the cell, wherein the guide sequence is capable of hybridizing to the target DNA. In some embodiments, the Cas nuclease or Cas nickase is guided to the target DNA via hybridization of the guide sequence and the target DNA. In some embodiments, the method is performed under conditions sufficient for the Cas nuclease or Cas nickase to generate a cleavage in the target DNA.

In some embodiments, one strand of the cleaved target DNA is a primer for a DNA polymerase. In some embodiments, the primer binding sequence of the first and/or the second polynucleotide is capable of binding to the primer. In some embodiments, the method does not comprise introducing an exogenous DNA polymerase into the cell. In some embodiments, the method is performed under conditions sufficient for an endogenous DNA polymerase of the cell to bind the primer and extend the sequence of interest. In some embodiments, the extending comprises synthesizing a DNA strand complementary to the sequence of interest to form a double-stranded sequence comprising the sequence of interest. In some embodiments, the method does not comprise introducing an exogenous DNA ligase into the cell. In some embodiments, the method is performed under conditions sufficient for an endogenous DNA ligase of the cell to ligate the sequence of interest to the cleaved target DNA.

In some embodiments, the method comprises introducing an exogenous DNA polymerase into the cell. Exogenous DNA polymerases are described herein. In some embodiments, the exogenous DNA polymerase synthesizes a DNA strand complementary to the sequence of interest. In some embodiments, the method comprises introducing an exogenous DNA ligase into the cell. In some embodiments, the exogenous DNA ligase ligates the sequence of interest to the cleaved target DNA. In some embodiments, the sequence of interest is a single-stranded sequence that is converted to a double-stranded sequence by a DNA repair pathway of the cell.

In some embodiments, the method further comprises contacting the cell with a DNA-dependent protein kinase (DNA-PK) inhibitor. In some embodiments, inhibition of DNA-PK reduces NHEJ and promotes HDR. In some embodiments, the cell is contacted with the DNA-PK inhibitor prior to being contacted with the composition described herein. In some embodiments, the DNA-PK inhibitor is AZD7648.

In some embodiments, the composition, polynucleotide, and/or fusion protein described herein are introduced into the cell via transfection. Transfection methods are further described herein. In some embodiments, the polynucleotides of the present disclosure, e.g., comprising a guide sequence, Cas-binding region, DNA template sequence, or any combination thereof, are transfected into the cell. In some embodiments, the polynucleotides are on one or more vectors.

In some embodiments, the proteins of the present disclosure, e.g., fusion protein, Cas nuclease, Cas nickase, DNA polymerase, and/or DNA ligase, are introduced into the cell via one or more polynucleotides encoding the protein. In some embodiments, the one or more polynucleotides are on one or more vectors. In some embodiments, the method comprises introducing multiple proteins into the cell, e.g., any combination of a fusion protein, Cas nuclease, Cas nickase, DNA polymerase, and DNA ligase. In some embodiments, the method comprises introducing a Cas nuclease, Cas nickase, or fusion protein and a DNA polymerase into the cell. In some embodiments, the method comprises introducing a Cas nuclease, Cas nickase, or fusion protein and a DNA ligase into the cell. In some embodiments, the method comprises introducing: a Cas nuclease, Cas nickase, or fusion protein; a DNA polymerase; and a DNA ligase into the cell. In some embodiments, the polynucleotides encoding the multiple proteins are on a single vector. In some embodiments, the polynucleotides encoding the multiple proteins are on more than one vector.

In some embodiments, the components of the composition described herein are on a single vector. In some embodiments, the components of the composition described herein are on more than one vector. In some embodiments, the method comprises transfecting one or more vectors into the cell, wherein the one or more vectors comprises: one or more polynucleotides encoding a fusion protein, Cas nuclease, or Cas nickase; and one or more polynucleotides comprising the guide sequence, Cas-binding region, DNA template sequence. In some embodiments, the method comprises transfecting a first vector and a second vector into the cell, wherein the first vector comprises a polynucleotide that encodes a fusion protein, Cas nuclease, or Cas nickase; and wherein the second vector comprises one or more polynucleotides that comprises the guide sequence, Cas-binding region, DNA template sequence. In some embodiments, the method comprises transfecting a single vector into the cell, wherein the single vector comprises: (i) one or more polynucleotides encoding a fusion protein, Cas nuclease, or Cas nickase; and (ii) one or more polynucleotides comprising the guide sequence, Cas-binding region, DNA template sequence.

In some embodiments, the composition, polynucleotide, and/or fusion protein described herein are introduced into the cell via a delivery particle. In some embodiments, the components of the composition are delivered in a single delivery particle. In some embodiments, the components of the composition are delivered in multiple delivery particles. Delivery particles can be used to deliver exogenous biological materials such as, e.g., polynucleotides and proteins described herein. In some embodiments, the delivery particle is a solid, a semi-solid, an emulsion, or a colloid. In some embodiments, the delivery particle is a lipid-based particle, a liposome, a micelle, a vesicle, or an exosome. In some embodiments, the delivery particle is a nanoparticle. Delivery particles are further described, e.g., in US 2011/0293703, US 2012/0251560, US 2013/0302401, U.S. Pat. Nos. 5,543,158, 5,855,913, 5,895,309, 6,007,845, and 8,709,843.

In some embodiments, the composition, polynucleotide, and/or fusion protein described herein are introduced into a cell via a vesicle. In some embodiments, the components of the composition are delivered in a single vesicle. In some embodiments, the components of the composition are delivered in multiple vesicles. In some embodiments, the vesicle comprises an exosome or a liposome. Engineered vesicles for delivery of exogenous biological materials into target cells are described, e.g., in US 2008/0234183; US 2019/0167810; US 2020/0207833; and Alvarez-Erviti et al., Nat Biotechnol 29:341 (2011).

All references cited herein, including patents, patent applications, papers, textbooks and the like, and the references cited therein, to the extent that they are not already, are hereby incorporated herein by reference in their entirety.

### EXAMPLES

### Example 1. Evaluation of Polynucleotides for Targeted Insertion

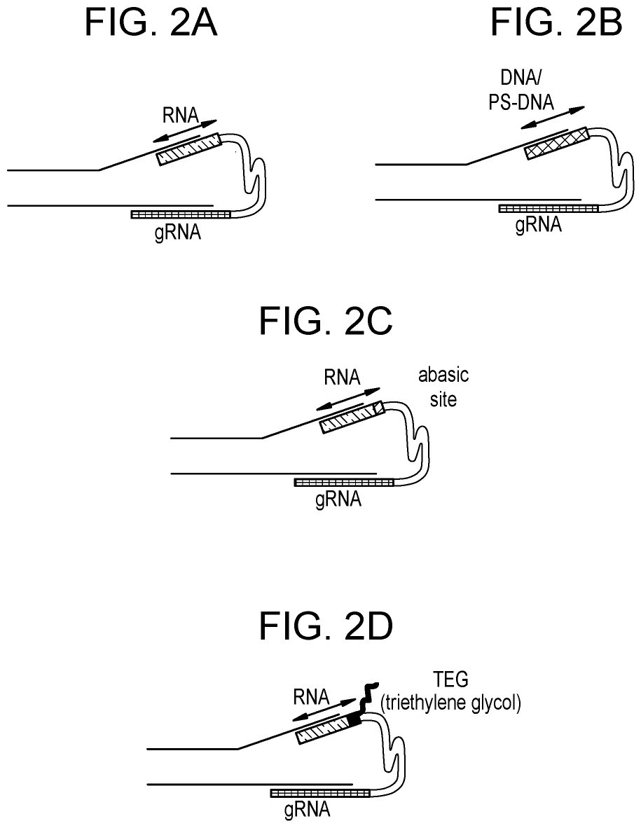

The S. pyogenes Cas9 protein (SpCas9) alone or SpCas9 fused to a reverse transcriptase (SpCas9-RT) were tested for in vivo targeted insertion with guide polynucleotides (referred to herein as “springRNA”) that comprised an RNA guide sequence targeting the AAVS1 locus, tracrRNA for binding SpCas9, and a template sequence at the 3′ end. Five springRNA constructs were prepared:
(1) RNA-only springRNA—a polynucleotide consisting of RNA nucleotides and comprising the RNA guide sequence, tracrRNA, a 6-bp insert sequence (AATATG), and a primer binding sequence (see FIG. 2A);
(2) springRNA with DNA tail (“DNA”)—same sequence as the RNA-only springRNA, and includes all RNA nucleotides except for the insert sequence and primer binding sequence, which consist of DNA nucleotides (see FIG. 2B);
(3) springRNA with DNA tail and phosphorothioate bonds (“PS-DNA”)—same as the springRNA with DNA insert, except that the insert sequence and primer binding sequence contain phosphorothioate bonds instead of phosphodiester bonds (see FIG. 2B);
(4) springRNA with abasic site—same as the RNA-only springRNA and consists of RNA nucleotides, except that the third base of in the insert sequence is replaced by an abasic site, the dSpacer nucleotide 1′2′-dideoxyribose (see FIG. 2C);
(5) springRNA with TEG linker—same as the RNA-only springRNA and consists of RNA nucleotides, except that the third base of the insert sequence is covalently attached to a triethylene glycol (TEG) (see FIG. 2D).

HEK293T cells were transfected, using FUGENEHD®, with a plasmid expressing either SpCas9 or SpCas9-RT. After twenty-four hours, the cells were further transfected, using LIPOFECTAMINE™ RNAiMAX, with 2 pmol of a springRNA construct listed above.

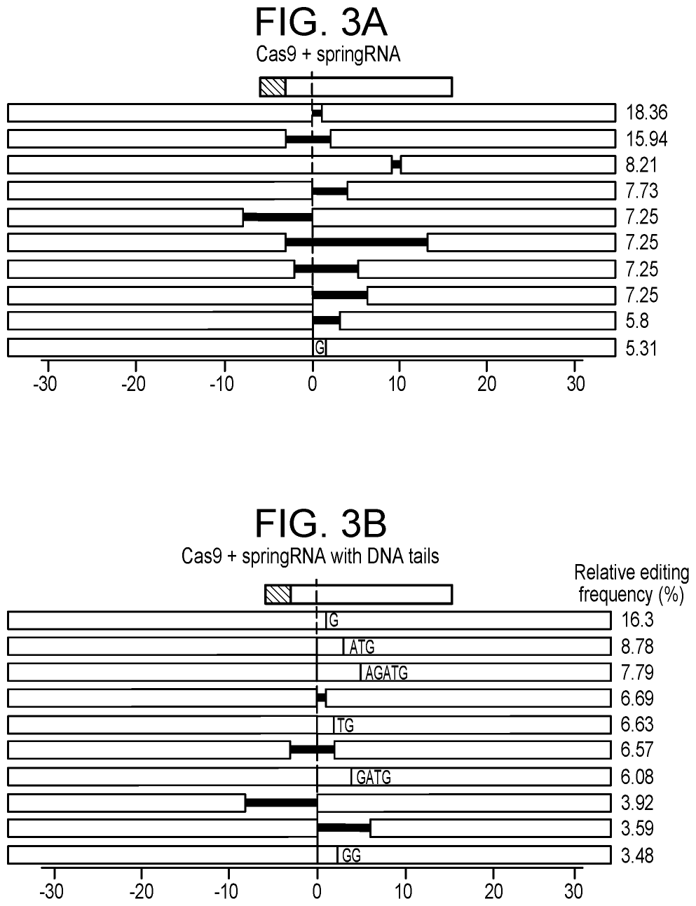

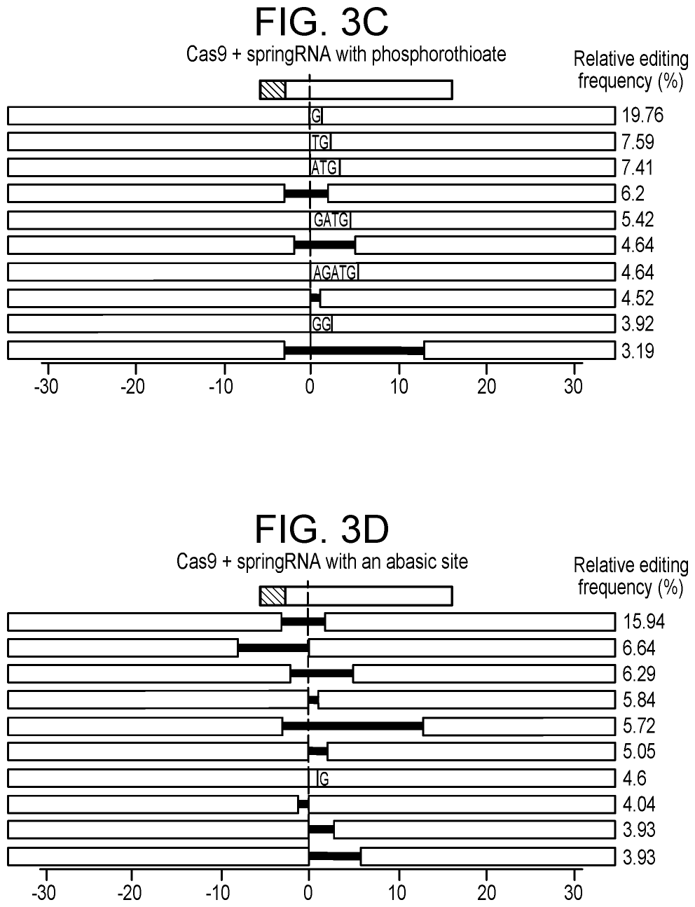

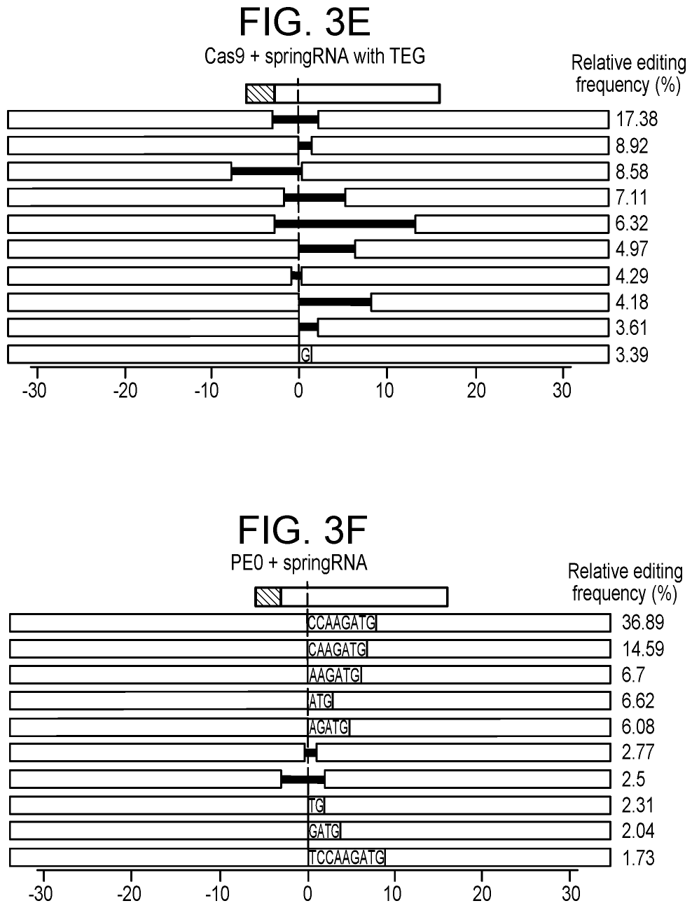

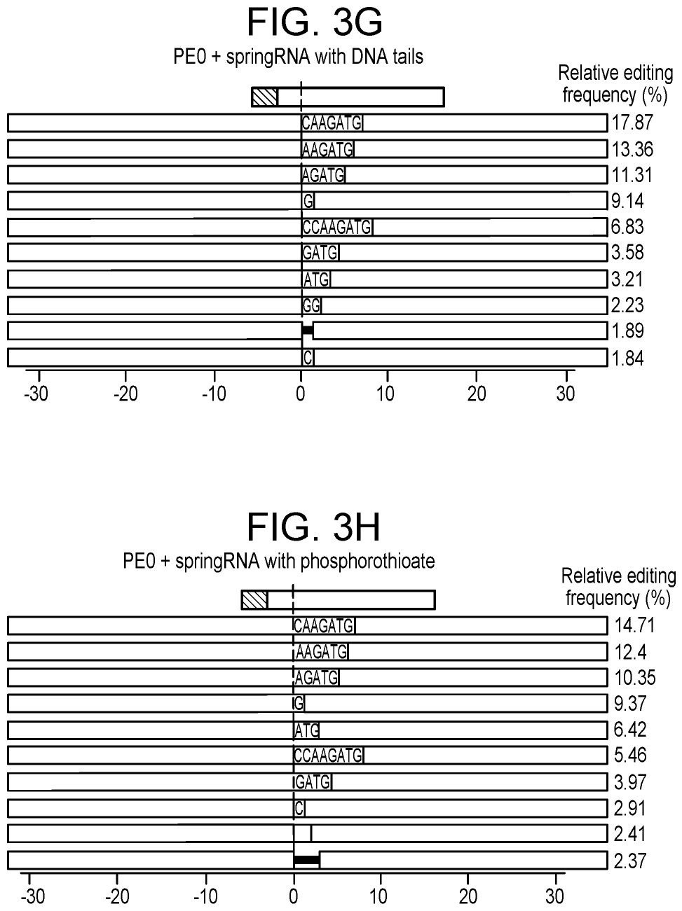

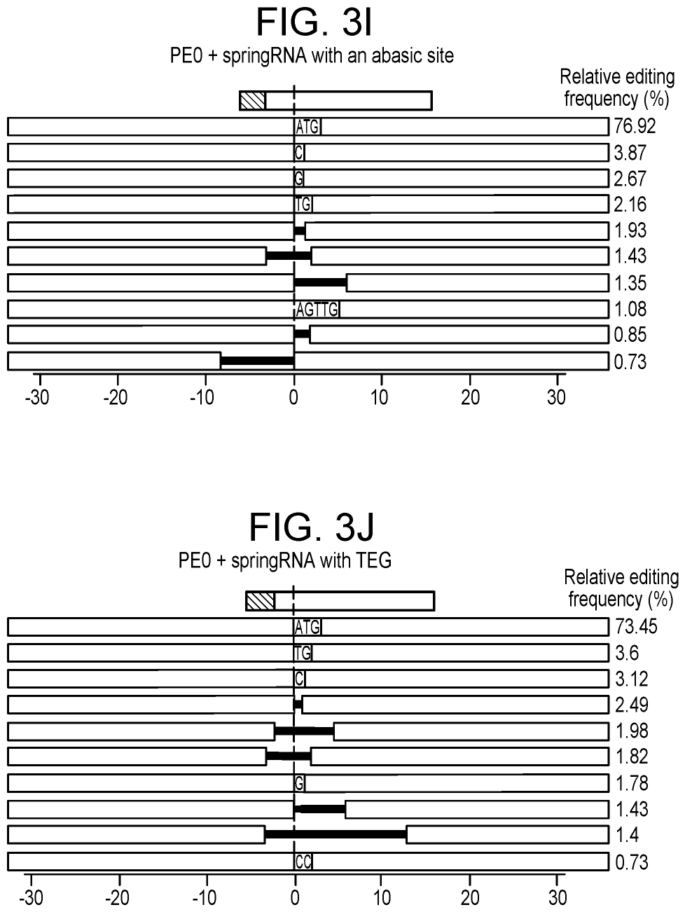

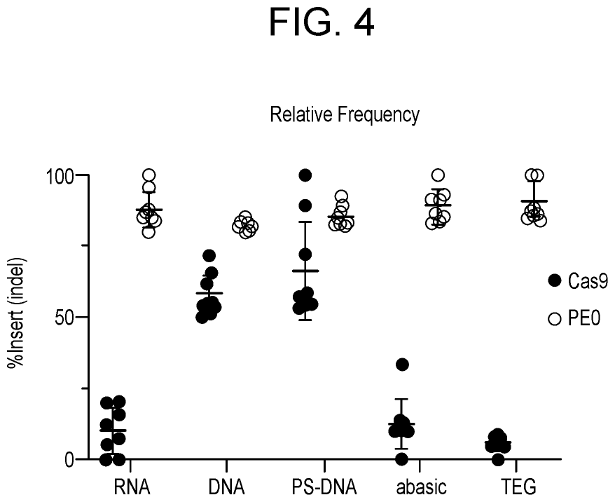

The cells were harvested 48 hours following transfection of the springRNA. The AAVS1 locus was amplified by PCR and sequenced using an Illumina sequencing platform. The results are shown in FIGS. 3 and 4. Panels A-E of FIG. 3 show targeted insertions with SpCas9 and each of the springRNA constructs above. Panels F-J of FIG. 3 show targeted insertions with SpCas9-RT (“PE0”) and each of the springRNA constructs above. FIG. 4 shows the relative frequency of insertions by SpCas9 only or SpCas9-RT in combination with each of the springRNA constructs above.

The results in FIGS. 3 and 4 indicate that co-transfection of a springRNA containing a DNA tail or DNA tail with phosphorothioate bonds (i.e., insert and primer binding sequences with DNA nucleotides, as described for constructs (2) and (3) above) with the SpCas9-expressing plasmid led to targeted insertion of the insert sequence, even in the absence of a reverse transcriptase fused to the SpCas9. Thus, endogenous cellular factors, e.g., endogenous cellular DNA polymerases, are capable of using the insert sequence as a template for DNA synthesis. Thus, targeted insertions can be performed using Cas9 only and a springRNA containing a DNA insert, i.e., DNA insert sequence and DNA primer binding sequence.

### Example 2. Further Evaluation of Polynucleotides for Targeted Insertion

The RNA-only springRNA, springRNA with DNA tail, and springRNA with DNA tail and phosphorothioate bonds (PS-DNA) were further tested with SpCas9 only or SpCas9-RT fusion. The springRNA constructs contained a guide sequence targeting AAVS1 and SpCas9 tracrRNA sequence as in Example 1. Sequences of the springRNA are provided in Table 4. The insert sequence and primer binding sequence are underlined; double-underline represents DNA nucleotides. The springRNAs were synthesized by Agilent.

TABLE 4
springRNA Sequences

| | |
|---|---|
| RNA-only | mG*mU*GGCCCCACUGUGGGGUGGGUUUUAGAGCUAGA |
| springRNA | AAUAGCAAGUUAAAAUAAGGCUAGUCCGUUAUCAACUU |
| | GAAAAAGUGGGACCGAGUCGGUCCAAGAUGCCCCACAG |
| | UGGGGCCACUmA*mG* |
| | (SEQ ID NO: 1) |
| springRNA | mG*mU*GGCCCCACUGUGGGGUGGGUUUUAGAGCUAGA |
| with DNA | AAUAGCAAGUUAAAAUAAGGCUAGUCCGUUAUCAACUU |
| tail | GAAAAAGUGGGACCGAGUCGGUCCdAdAdGdAdTdGdC |
| | dCdCdCdAdCdAdGdTdGdGdGdGdCdCdAdCdTdAdG |
| | (SEQ ID NO: 2) |
| springRNA | mG*mU*GGCCCCACUGUGGGGUGGGUUUUAGAGCUAGA |
| with PS-DNA | AAUAGCAAGUUAAAAUAAGGCUAGUCCGUUAUCAACUU |
| tail | GAAAAAGUGGGACCGAGUCGGUCCdAdAdGdAdTdGdC |
| | dCdCdCdAdCdAdGdTdGdGdGdGdCdCdAdCdTdA*d |
| | G* |
| | (SEQ ID NO: 3) |

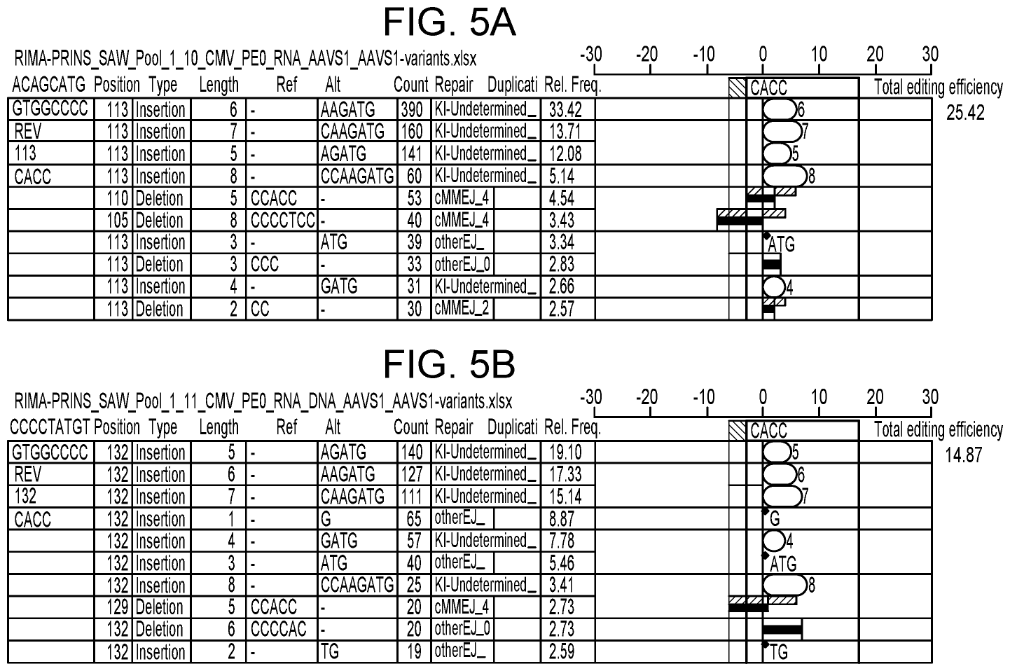

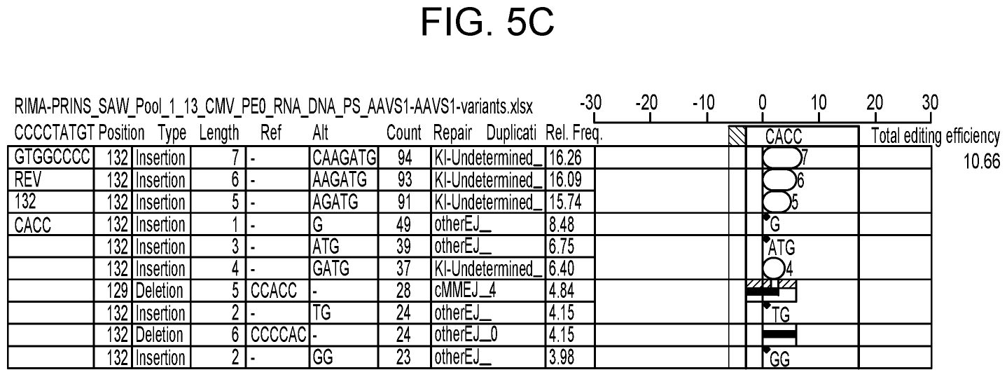

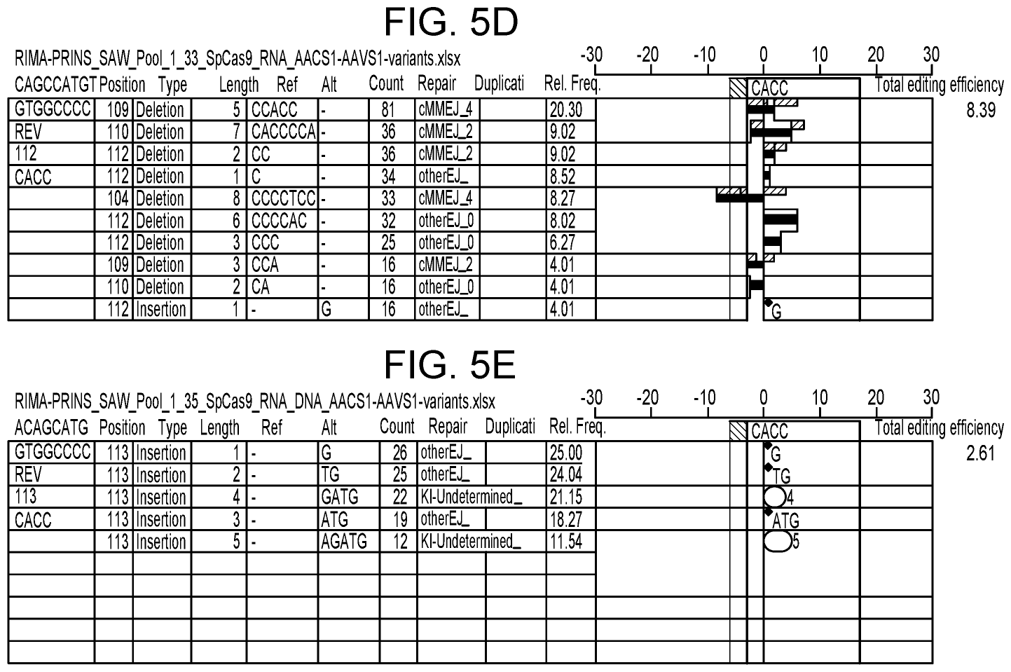

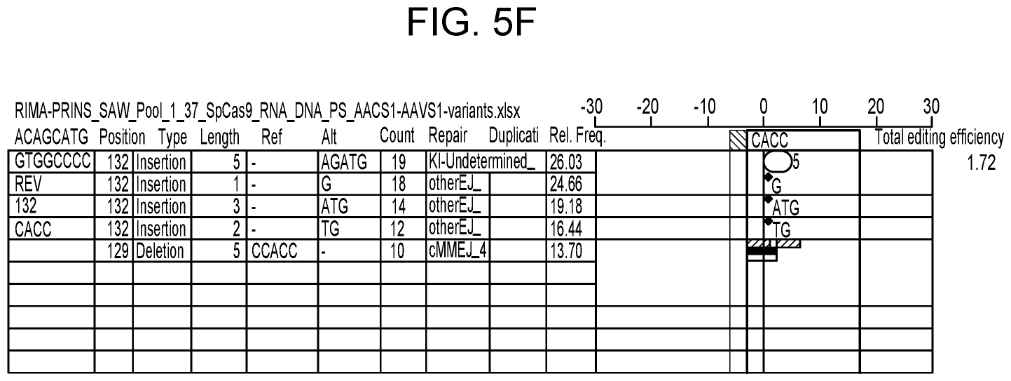

SpCas9 or SpCas9-RT and springRNA were transfected into cells, the cells were harvested, and the AAVS1 locus was analyzed as described for Example 1. Results are shown in FIGS. 5A-5F. FIGS. 5A-5C show the targeted insertions with SpCas9-RT (“PE0”) and RNA-only springRNA (FIG. 5A), springRNA with DNA tail (FIG. 5B), and springRNA with PS-DNA (FIG. 5C). FIGS. 5D-5F show the targeted insertions with SpCas9 and RNA-only springRNA (FIG. 5D), springRNA with DNA tail (FIG. 5E), and springRNA with PS-DNA (FIG. 5F).

The results indicate that the combination of SpCas9 and springRNA containing a DNA tail or PS-DNA tail with PS-DNA, i.e., insert and primer binding sequences with DNA nucleotides, achieved a similar insertion pattern as the combination of SpCas9-RT and springRNA.

### Example 3. In Vitro Assay

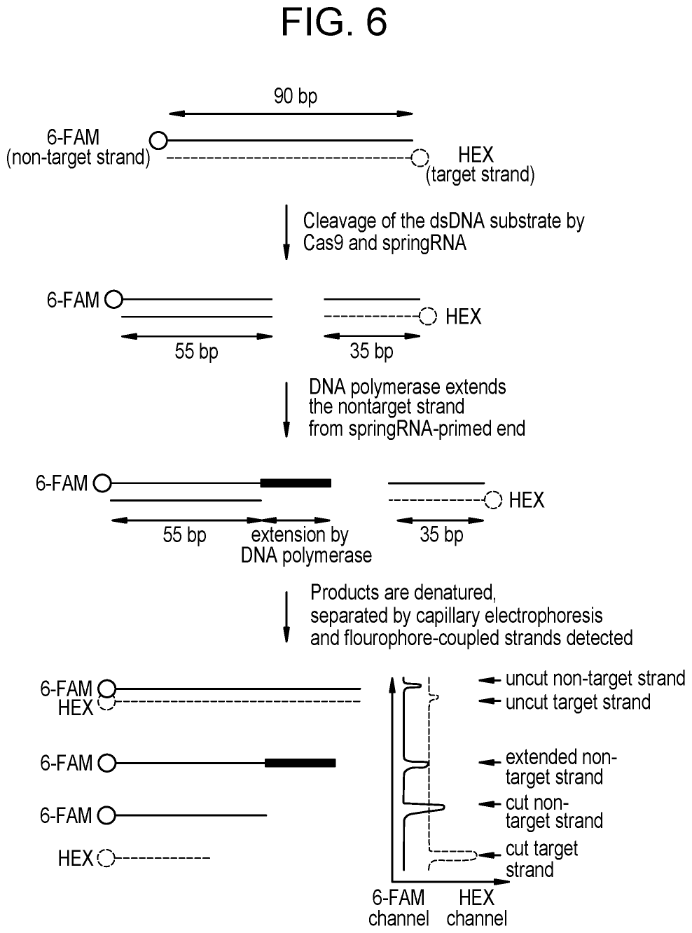

An in vitro assay was performed to evaluate the role of DNA polymerase in targeted insertions using Cas9. An overview of the assay is shown in FIG. 6. In the in vitro assay, two complementary DNA strands are labeled with different fluorophores (6 FAM-labeled non-target strand and HEX-labeled target strand). A guide sequence is designed such that Cas9 cleavage generates two strands of different lengths. A DNA polymerase extends the 6 FAM-labeled non-target strand hybridized to the primer binding sequence of the springRNA. The products are denatured and separated by capillary electrophoresis, and the fluorophore-coupled strands are detected.

A synthetic target DNA substrate was prepared by annealing two complementary strands: a 6 FAM-labeled non-target strand and HEX-labeled target strand. Cas9 and springRNA were mixed at equimolar ratios, and ribonucleoprotein complexes were formed at room temperature. The Cas9:springRNA complexes were added to the synthetic target DNA substrate at a 15-fold molar excess. DNA polymerase (either the Bst 3.0 DNA polymerase or the Klenow fragment of E. coli DNA Pol I, in its optimized buffer) was added to the reaction mixture and incubated for 1 hour at 37° C. The reactions were halted by Proteinase K digestion. 1 μL of the reaction mixture was added to HIDI™ formamide and denatured for 3 minutes at 95° C. The denatured products were resolved and analyzed by capillary electrophoresis on a 3730 DNA Analyzer (Applied Biosystems).

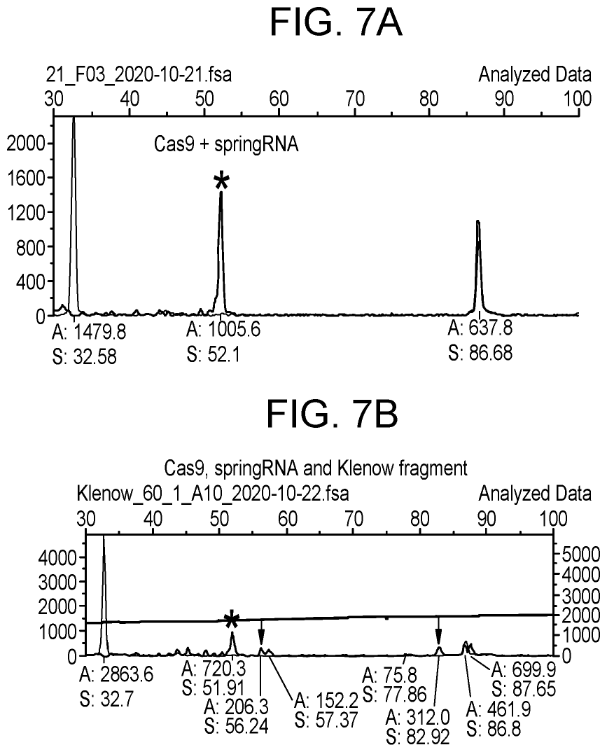

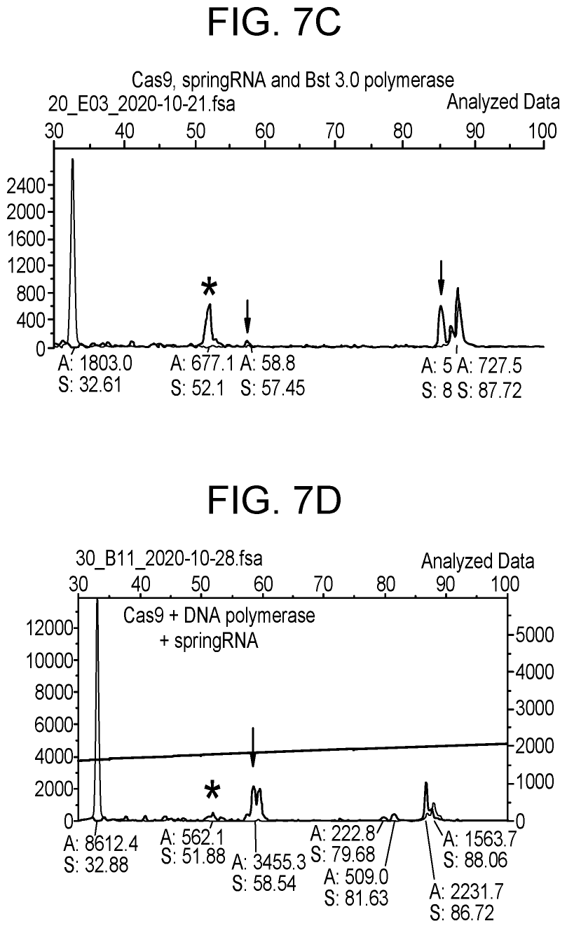

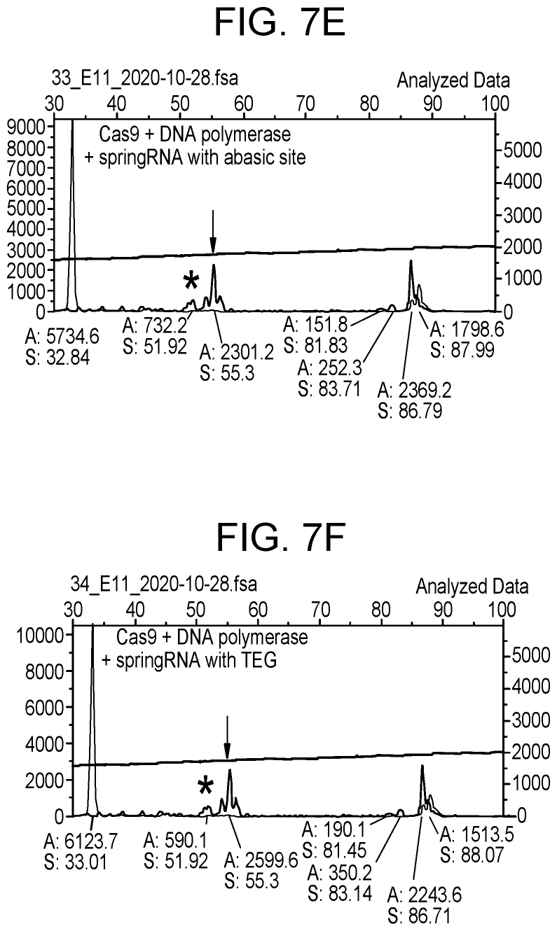

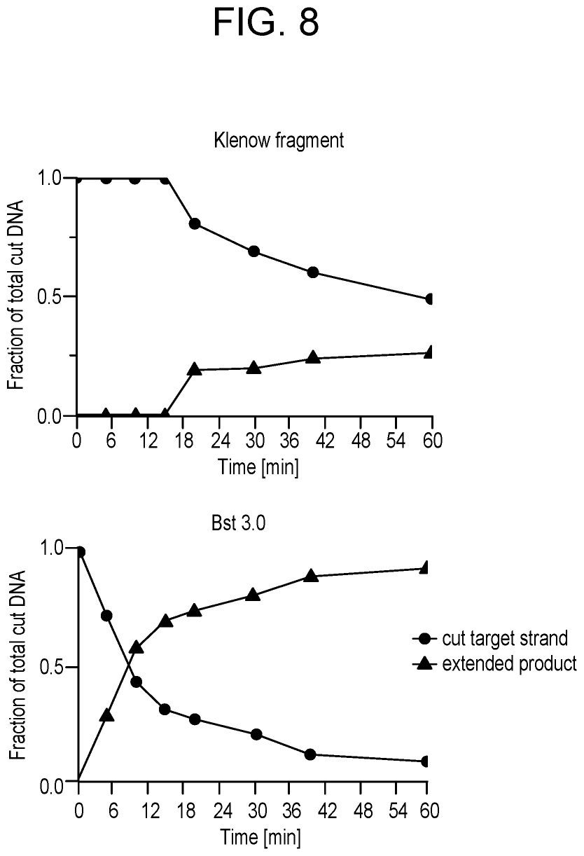

Results are shown in FIGS. 7A-7F. The blue traces correspond to the non-target strand, and green traces correspond to the target strand. The asterisks indicate the cleaved synthetic target DNA substrate, and black arrows indicate extension products by the DNA polymerase. FIG. 7A shows the assay results with Cas9 and springRNA. FIG. 7B shows the assay results with Cas9, Klenow fragment, and springRNA. FIGS. 7C-7F show the assay results with Cas9, Bst 3.0 polymerase, and: springRNA (FIGS. 7C-7D), springRNA with abasic site (FIG. 7E), or springRNA with TEG (FIG. 7F). The in vitro assay results indicate that DNA polymerase can perform targeted insertion without being fused to a Cas9. FIG. 8 shows the kinetics of the extension reactions by the Klenow fragment or Bst 3.0 polymerase.

### Example 4. Proximal Ligation Upstream gRNA

The following Examples 5-19 relate to proximal ligation upstream gRNA (“plugRNA”).

As described throughout the present disclosure and Examples, a plugRNA includes a gRNA molecule with a polynucleotide appended to the 3′ end. The polynucleotide comprises two elements: a first hybridization region (also referred to in the Examples and Figures as the “landing pad”), and a primer binding sequence (PBS) that hybridizes to the sequence upstream of the cut induced by Cas protein. The plugRNA can be a single molecule, or can comprise multiple polynucleotides that anneal to each other, forming a binary, tertiary or quaternary complex. Any element of the plugRNA can comprise DNA, RNA, LNA, PNA, or a chemically modified nucleotide.

As described throughout the present disclosure and Examples, the Cas protein can be any Cas protein that is able to bind plugRNA. The Cas protein can be any type I or II Cas protein, including but not restricted to Cas9, Cas12, Cascade complexes. The Cas protein can be fused to a fluorescent tag, a DNA polymerase, a reverse transcriptase, a DNA ligase (for example, but not restricted to: T4 DNA ligase, Paramecium bursaria Chlorella virus 1 (PBCV-1) ligase, Mycobacterium Ligase D (LigD), human Ligase 1, human Ligase 3, human Ligase 4), a ligase adaptor protein (for example, but not limited to: XRCC1, XRCC4, PCNA), a single stranded DNA binding protein (such as RPA1/2/3, SSBP1-4), a DNA repair protein (TDP1, aprataxin, topoisomerase I), or a combination thereof.

The Examples herein further refer to a “Donor” polynucleotide, also referred to in the present disclosure as a “second polynucleotide.” The donor polynucleotide comprises a second hybridization region, which is complementary to the first hybridization region in the plugRNA; a sequence of interest (also referred to in the Examples and Figures as the “insert”), and a homology sequence, which is complementary or substantially complementary to a sequence proximal to the target cleavage site. The donor design can omit any of these elements, and can comprise two or more polynucleotides hybridized to each other, or can be paired with another polynucleotide to form double stranded regions. Any section of the donor can comprise DNA, RNA, PNA, LNA, or a chemically modified nucleotide. The 5′ and 3′ ends of the donor can be phosphorylated, adenylated, phosphorothioated, etc.

The present disclosure provides a method of making a site-specific modification, also referred to herein as knock-in nucleotide guided (“KING”) editing. A general strategy for KING editing is shown in FIG. 10. Briefly, Cas9 forms a ternary (or higher order) complex with the plugRNA and donor as described herein. The Cas9 induces a double stranded break. The PBS of the plugRNA anneals to the target site, juxtaposing the donor to the break site. The donor can be directly ligated by the endogenous or exogenously provided DNA ligase (FIGS. 10A and 10B). Alternatively, donor can be integrated by a concerted action of a DNA ligases and the homology-based mechanisms (homologous recombination, single-strand annealing [SSA], microhomology mediated end joining [MMEJ], synthesis-dependent strand annealing [SDSA], DNA or RNA templated DNA repair). Once the donor is integrated into the target strand, the endogenous cellular machinery can resolve the intermediate, leading to insertion of the desired sequence into the target DNA.

### Example 5: Design of plugRNA and Donor

FIG. 9 depicts the general design of plugRNAs with donor, and relevant elements. plugRNA consists of a guide sequence (“spacer RNA targeting specific sequence”) (1), followed by gRNA scaffold that allow Cas9 protein binding and activity. The 3′ end of the spacer-gRNA scaffold extends into polynucleotide. This polynucleotide (made of DNA, RNA, modified in various chemical ways and in combinations thereof) consist of two elements: a first hybridization sequence (“landing pad”) (2), and primer binding sequence (3) that hybridizes to the sequence upstream of the cut induced by Cas protein. Nucleotides in immediate vicinity of the break (i.e. the junction between first hybridization sequence and primer binding sequence) are composed of DNA.

The first hybridization sequence hybridizes to the second hybridization sequence (“hybridization sequence”) (4) of the donor, juxtaposing the 5′ end of the donor to the break site. In this configuration three polynucleotides present a nicked nucleic acid, which is an exquisite substrate to DNA ligases.

Hybridization sequence can be phosphorylated, adenylated, covalently attached to protein or modified in chemically different ways. Donor further consists of the sequence of interest (“insert sequence”) (5) and homology sequence (6), which is homologous to the sequence proximal to the break site. Homology sequence is used to engage homology-based mechanisms (HR, SSA or MMEJ), potentially improving editing efficiency.

Both donor and plugRNAs can contain additional elements, omit some elements or be split in multiple oligonucleotides (see FIG. 11 and Example 7). For clarity, base pairing is not depicted between donors and plugRNA.

### Example 6: Strategy to Generate Precise Insertions Through Activity of Ligase

FIG. 10 depicts a general strategy for knock-in nucleotide guided (“KING”) editing. Cas protein, guided by the plugRNA can induce a cut at a specified DNA sequence. Through hybridization of the primer binding sequence (“PBS”) to the break site, the donor is juxtaposed to the break site. The donor can be ligated directly at the both 5′ and 3′ ends, or ligated at one end (here depicted at 5′) and integrated by homology-mediated mechanisms (HR, SDSA, SSA, alt-EJ and others) on the other end. The generated chimeric molecule is converted to dsDNA, integrating the desired sequence into the target site.

### Example 7: Examples of Different plugRNA:Donor Configurations

FIG. 11 depicts some of the strategies to perform KING editing. FIG. 11(A) depicts the donor containing all three elements: second hybridization sequence, the sequence of interest, and the homology sequence. FIG. 11(B) depicts donor without homology sequence. FIG. 11(C) depicts donor without the second hybridization sequence. FIG. 11(D) represents a standard donor, with a sequence complementary to the sequence of interest hybridized to form a double stranded region sequence of interest with both 5′ and 3′ overhangs. This configuration is termed “overhang.” FIG. 11(E) depicts a donor as in FIG. 11(A), but the plugRNA is composed of two nucleic acids. The sgRNA has a ˜30 nt long sequence added to the 3′ end. This 3′ sequence can pair with a complimentary polynucleotide, which is then followed by the first hybridization sequence and the primer binding sequence. This configuration is termed “split plugRNA” or “split system.” FIG. 11(F) depicts a configuration as in FIG. 11(A), but the hybridization sequence is shorter, generating a gap between the 3′ end of the primed target site and 5′ end of the donor. This configuration is termed “gap.” FIG. 11(G) is the same as in FIG. 11(A), but the donor has additional nucleotides at the 5′ end, generating a flap, that in some embodiments may engage FEN1 and other repair machinery. This configuration is termed “flap.” FIG. 11(H) is the same as in FIG. 11(B), but a sequence complementary to the sequence of interest is annealed, generating a blunt end on the 3′ end of the donor. This configuration is termed “blunt.” FIG. 11(I) the donor is split into two polynucleotides. The first donor strand comprises second hybridization sequence and a sequence of interest. The second sequence comprises the homology sequence and a sequence complementary to the sequence of interest. This configuration is termed “bridge.” In FIG. 11(J), the homology sequence is embedded in the plugRNA, immediately downstream of the gRNA scaffold. This homology sequence anneals to the 3′ side of the Cas-induced break. The donor comprises the sequence of interest flanked by two hybridization sequences: the second hybridization sequence and a further hybridization sequence, the second hybridization sequence complementary to the first hybridization sequence, and the further hybridization complementary to a region adjacent to the homology sequence. This configuration brings both 5′ and 3′ sides of the donor in proximity to the double strand break. This configuration is termed “dual hybridization.”

### Example 8: Proof of Concept In Vitro Assays

### Material and Methods

HEX-labeled DNA oligo was annealed to its complementary sequence at 200 nM in Annealing buffer (10 mM Tris pH 7.5, 50 mM NaCl) by denaturing at 95° C. for 2 minutes and then ramped down at 0.1° C./s to 4° C. plugRNAs were mixed in equimolar ratios with donor oligos (final concentration 1 μM in annealing buffer) and were annealed as above. Cas9 nuclease was dissolved in reaction buffer (1×T4 DNA ligase buffer, NEB) and mixed with an equimolar amount (final concentration 150 nM). Cas9-plugRNA-donor complexes were assembled by incubating for 10 minutes at ambient temperature. To assembled RNPs, fluorescent double stranded DNA substrate was added (10 nM final concentration). The reaction was incubated for 10 minutes at 37° C. to cut target DNA. After incubation, 200 U of T4 DNA ligase was added and reaction continued at 37° C. for one hour. The reaction was terminated by adding stop solution (final concentration: Proteinase K 0.1 mg/ml, 0.1% SDS, 12.5 mM EDTA) and incubated 30 min at 56° C. Reaction products were dissolved in HiDi formamide with GeneScan LIZ500, denatured at 95° C. and then resolved on capillary electrophoresis.

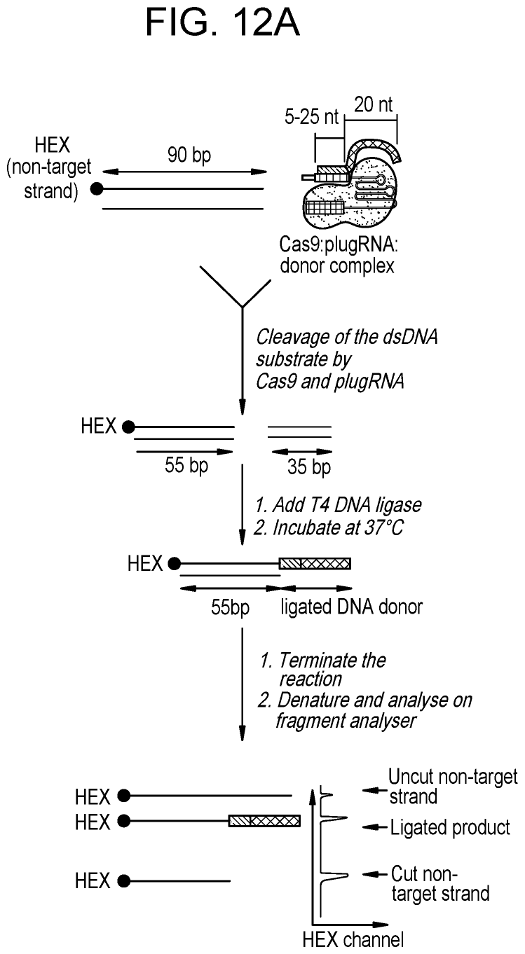

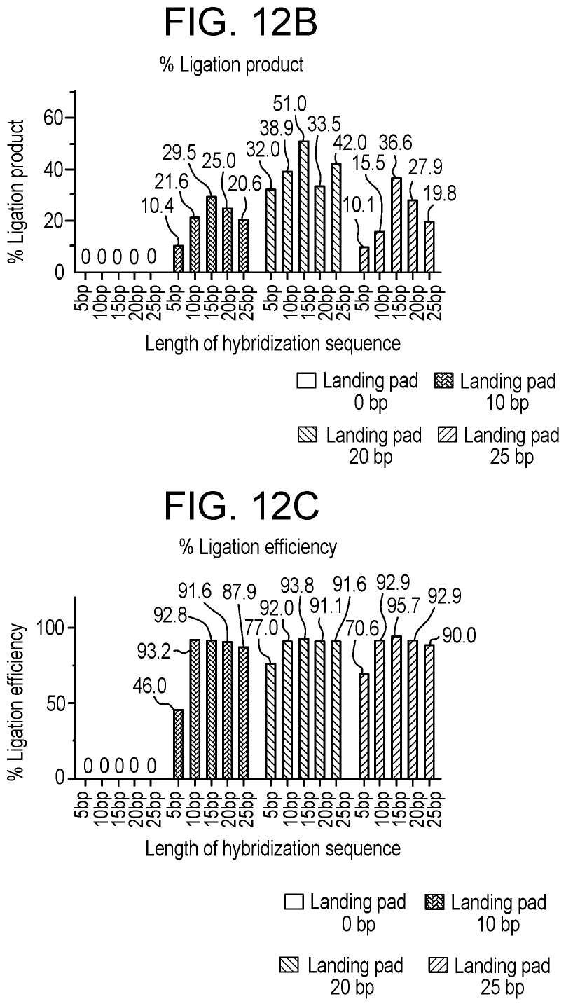

FIG. 12A represents the layout of the assay. FIG. 12B and FIG. 12C represent quantification of in vitro assays with combination of plugRNAs and donors with first and second hybridization sequences of different lengths, respectively. Two parameters were calculated: % ligation product, calculated as the percentage of total DNA detected in the assay corresponding to ligated DNA; and % ligation efficiency, calculated as the fraction of cut DNA that has been ligated. FIG. 12B shows that the ligation products are generated with high frequency, with maximal amount reached when plugRNA with landing pad of 20 bp and hybridization sequence 15 bp. No ligation was observed if the plugRNA did not contain landing pad, showing that annealing of the donor DNA to the plugRNA is required. FIG. 12C shows that regardless of the combination, ligation is extremely efficient, reaching >90% ligation efficiency.

### Example 9. In Vitro Ligation Using HeLa Nuclear Extracts

### Material and Methods

HEX-labeled DNA oligo was annealed to its complementary sequence at 200 nM in Annealing buffer (10 mM Tris pH 7.5, 50 mM NaCl) by denaturing at 95° C. for 2 minutes and then ramped down at 0.1° C./s to 4° C. plugRNAs were mixed in equimolar ratios with donor oligos (final concentration 1 μM in annealing buffer) and were annealed as above. Cas9 nuclease was dissolved in reaction buffer (50 mM Tris-HCl (pH 7.5), 0.5 mM magnesium acetate, 60 mM potassium acetate, and 0.1 mg/ml BSA) and mix with equimolar amount (final concentration 150 nM). Cas9-plugRNA-donor complexes were assembled by incubating for 10 minutes at ambient temperature. To assembled RNPs, fluorescent double stranded DNA substrate was added (10 nM final concentration). Reaction was incubated for 10 minutes at 37° C. to cut target DNA. During the incubation, HeLa nuclear extract (Ipracell) was premixed with dNTP and rNTP mix, and incubated for 10 minutes at room temperature. After incubation, premixed HeLa nuclear extract was added to the RNP/substrate mix (2.5 mg/ml HeLa nuclear extract, 5 mM ATP, 0.2 mM CTP, 0.2 mM GTP, 0.2 mM UTP, 0.2 mM dATP, 0.2 mM dCTP, 0.2 mM dGTP, 0.2 mM dUTP). Reaction was incubated further 1 hour at 37° C. Reaction was terminated by adding stop solution (final concentration: Proteinase K 0.1 mg/ml, 0.1% SDS, 12.5 mM EDTA) and incubated 30 min at 37° C. Reaction products we dissolved in HiDi formamide with GeneScan LIZ500, denatured at 95° C. and then resolved on capillary electrophoresis.

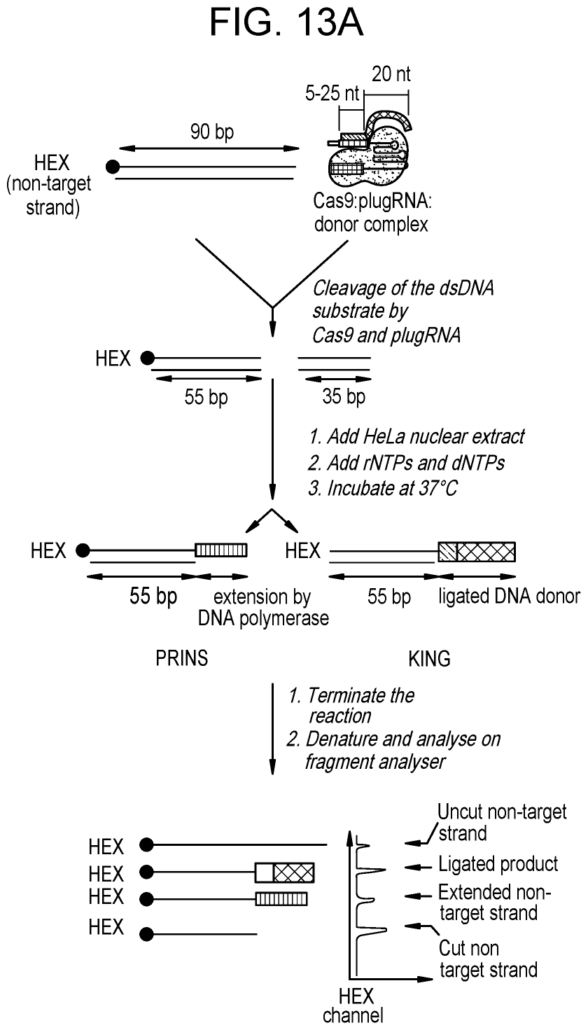

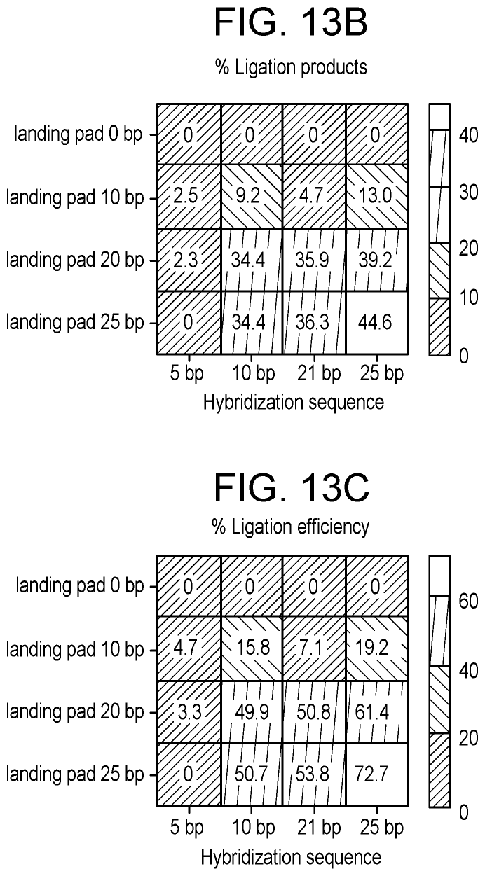

FIG. 13A represents the layout of the experiment. HeLa nuclear extract has been extensively used in biochemical studies as it retains the activity of most proteins and can be used to reconstitute various complex processes (including replication, repair and transcription). Further to using viral recombinant ligases, HeLa nuclear extract may provide insight into the feasibility of KING editing using enzymatic activities present in human cells.

FIGS. 13B and 13C depict quantification of the in vitro assays with HeLa nuclear extract. As in FIG. 12 and Example 8, both the abundance of products (as a fraction of total DNA corresponding to ligation products) and the efficiency of ligation (as a proportion of cut DNA converted to ligated products) were quantified. The heat map of results in FIGS. 13B and 13C demonstrated that KING editing is efficient. As the size of the first hybridization region (“landing pad”) and second hybridization region (“hybridization sequence”) increases, more abundant ligation products were observed. It was also observed that the ligation was exceptionally efficient when HeLa nuclear extracts are used.

### Example 10. Reconstitution of KING Editing Using Nuclear Extract

### Material and Methods

plugRNAs were mixed in equimolar ratios with donor oligos (final concentration 1 μM in annealing buffer) and were annealed by denaturing at 95° C. for 2 minutes and then ramped down at 0.1° C./s to 4° C. Cas9 nuclease was dissolved in reaction buffer (50 mM Tris-HCl (pH 7.5), 0.5 mM magnesium acetate, 60 mM potassium acetate, and 0.1 mg/ml BSA) and mix with equimolar amount (final concentration 150 nM). Cas9-plugRNA-donor complexes were assembled by incubating for 10 minutes at ambient temperature. To assembled RNPs, plasmid containing target AAVS1 sequence was added (10 nM final concentration). Reaction was incubated for 10 minutes at 37° C. to cut target DNA. During the incubation, HeLa nuclear extract (Ipracell) was premixed with dNTP and rNTP mix, and incubated for 10 minutes at room temperature. After incubation, premixed HeLa nuclear extract was added to the RNP/substrate mix (2.5 mg/ml HeLa nuclear extract, 5 mM ATP, 0.2 mM CTP, 0.2 mM GTP, 0.2 mM UTP, 0.2 mM dATP, 0.2 mM dCTP, 0.2 mM dGTP, 0.2 mM dUTP). Reaction was incubated further 1 hour at 37° C. Half a microlitre of the reaction was used as a template in PCR reaction to amplify targeted AAVS1 sequence. PCR products were resolved using capillary electrophoresis or were submitted for next-generation sequencing using NextSeq500. Sequencing results were aligned to reference sequence and edits analyzed by RIMA (See, e.g., Taheri-Ghahfarokhi et al., PMID: 30032200).

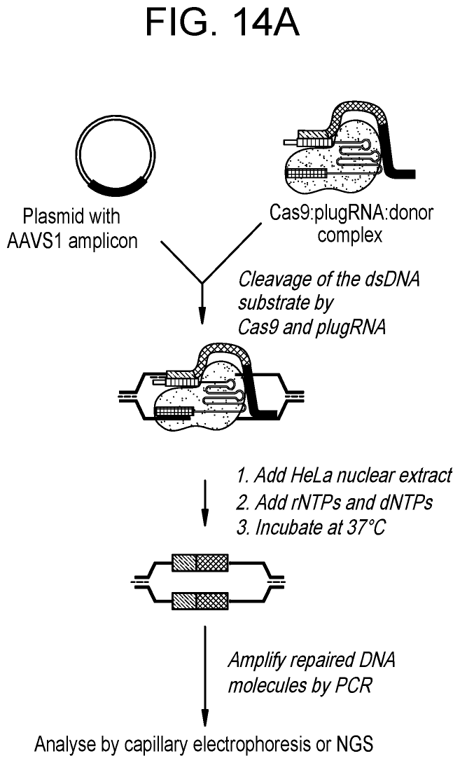

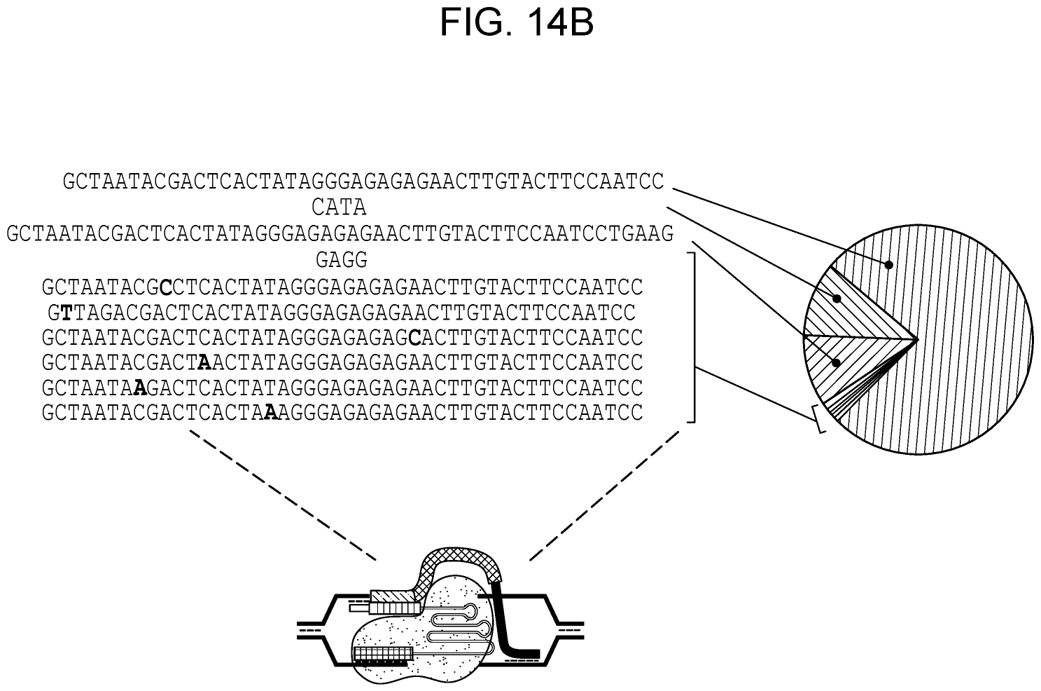

FIG. 14A shows the layout of the protocol. FIG. 14B depicts representative result after NGS. Top 10 insertion variants detected at the recovered AAVS1 target site are represented. The top variant comprising >75% edited reads corresponded to the predicted and desired sequence (hybridization sequence [dotted] and insert [cross hatched]). A shortened insertion was also detected at much lower frequency. Insertion of desired sequence with errors was also detected at frequencies lower than 0.5%. These results showed that the entire KING reaction could be reconstituted, leading to insertion of a desired sequence into target site with high precision and fidelity.

### Example 11. Insertion Efficiencies in Reconstituted KING Assay with Various Substrates

### Material and Methods

plugRNAs were mixed in equimolar ratios with donor oligos (final concentration 1 μM in annealing buffer) and were annealed by denaturing at 95° C. for 2 minutes and then ramped down at 0.1° C./s to 4° C. Cas9 nuclease was dissolved in reaction buffer (50 mM Tris-HCl (pH 7.5), 0.5 mM magnesium acetate, 60 mM potassium acetate, and 0.1 mg/ml BSA) and mix with equimolar amount (final concentration 150 nM). Cas9-plugRNA-donor complexes were assembled by incubating for 10 minutes at ambient temperature. To assembled RNPs, plasmid containing target AAVS1 sequence was added (10 nM final concentration). Reaction was incubated for 10 minutes at 37° C. to cut target DNA. During the incubation, HeLa nuclear extract (Ipracell) was premixed with dNTP and rNTP mix, and incubated for 10 minutes at room temperature. After incubation, premixed HeLa nuclear extract was added to the RNP/substrate mix (2.5 mg/ml HeLa nuclear extract, 5 mM ATP, 0.2 mM CTP, 0.2 mM GTP, 0.2 mM UTP, 0.2 mM dATP, 0.2 mM dCTP, 0.2 mM dGTP, 0.2 mM dUTP). Reaction was incubated further 1 hour at 37° C. Half a microlitre of the reaction was used as a template in PCR reaction to amplify targeted AAVS1 sequence. PCR products were resolved using capillary electrophoresis or were submitted for next-generation sequencing using NextSeq500. Sequencing results were aligned to reference sequence and edits analysed by RIMA (PMID: 30032200).

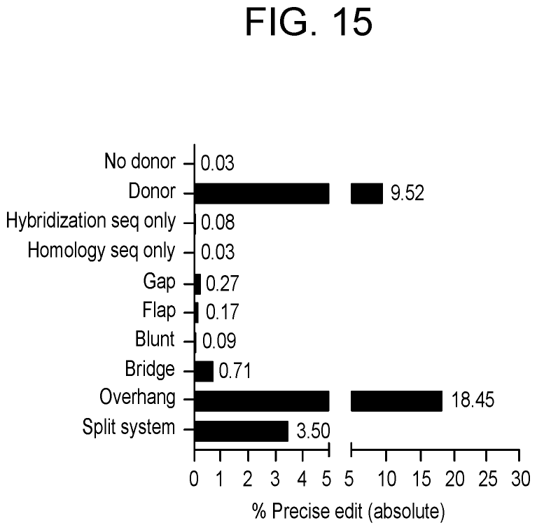

FIG. 15 depicts frequencies of desired insert (assayed by NGS) when using different strategies (see FIG. 11). As shown in FIG. 15, presence of hybridization and homology sequences and/or a double-stranded donor led to higher frequency of precise KING editing. plugRNA:donor complexes with different configurations of these elements were able to generate insertions at varying levels.

### Example 12. Optimization of KING Editing Components for Precise Insertion

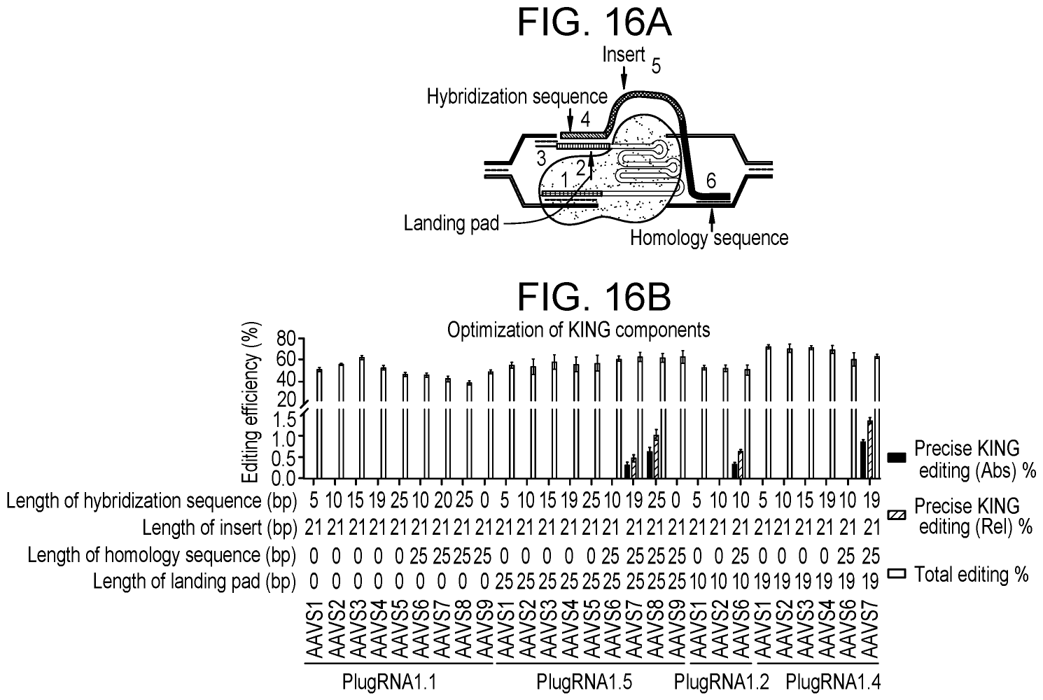

In this Example, various KING editing components were tested (see FIG. 16A) for ability to generate more precise insertions.

### Methods:

HEK293T cells were transfected with plasmid driving the expression of Cas9 with FugeneHD. 24 hours later plugRNAs and ssDNA donors were assembled using the following conditions: Denature at 95° C. for 2 minutes and then ramp down to 4° C. at the rate of 0.1° C./s. The cells were transfected with 2 pmol of assembled plugRNAs and ssDNA donors with RNAiMAX. Different length of first hybridization regions (“landing pads”) for plugRNA and different length of second hybridization region (“hybridization sequence”) and homology sequence for ssDNA donors were tested targeting various AAVS sites. Genomic DNA was harvested from transfected cells after 48 hours and analyzed after amplification by ILLUMINA next generation sequencing.

### Results:

It was observed that the landing pad, homology sequence, and hybridization sequence were used for introducing precise insertion by KING editing using ssDNA donors. See FIG. 16B. The intended precise insertions were detected using plugRNA1.2 (+AAVS6), plugRNA1.4 (+AAVS7) and plugRNA1.5 (+AAVS7 or +AAVS8).

### Example 13. KING Editing with ssDNA Donors

In this Example, ssDNA donors for KING editing and various lengths for homology sequences were tested.

### Methods:

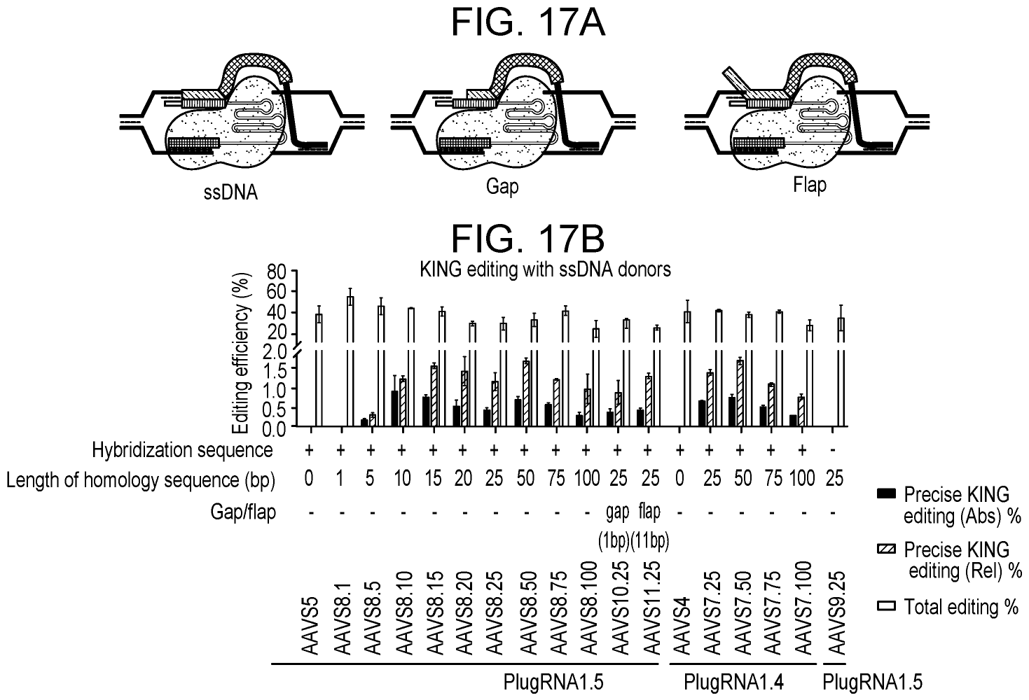

HEK293T cells were transfected with plasmid driving the expression of Cas9 with FugeneHD. 24 hours later, plugRNAs and ssDNA donors were assembled using the following conditions: Denature at 95° C. for 2 minutes and then ramp down to 4° C. at the rate of 0.1° C./s. The cells were transfected with 2 pmol of assembled plugRNAs and ssDNA donors with RNAiMAX. Different length of homology sequence for ssDNA donors or ssDNAs with gap or flap (FIG. 17A) were tested. Genomic DNA was harvested from transfected cells after 48 hours and analyzed after amplification by ILLUMINA next generation sequencing.

### Results:

It observed that the absolute and relative precise editing efficiency of KING was further improved by optimizing the length of homology sequence for the ssDNA donor. See FIG. 17B. For plugRNA1.5, the highest absolute precise editing efficiency of KING editing was detected using ssDNA donors with 10 or 50 bp homology sequence. For plugRNA1.4, the highest absolute precise editing efficiency of KING editing was detected using ssDNA donors with 50 bp homology sequence. FIG. 17B further demonstrated that precise insertion can be introduced by KING editing using gap- or flap-containing donors.

### Example 14. KING Editing with dsDNA Donors

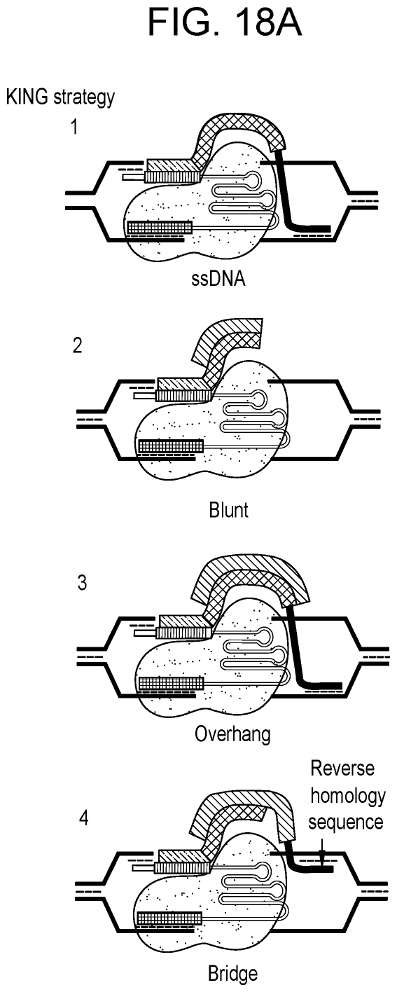

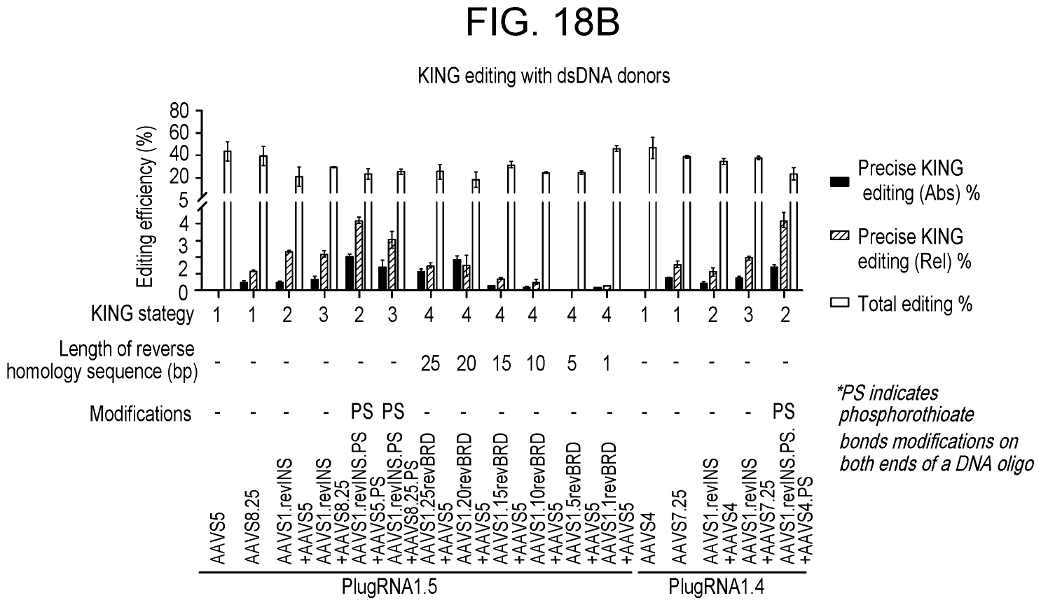

In this Example, KING editing was tested with dsDNA donors with the various configurations shown in FIG. 18A.

### Methods:

HEK293T cells were transfected with plasmid driving the expression of Cas9 with FugeneHD. 24 hours later plugRNAs and ssDNA donors were assembled using the following conditions: Denature at 95 C for 2 minutes and then ramp down to 4° C. at the rate of 0.1° C./s. The cells were transfected with 2 pmol of assembled plugRNAs and ssDNA/dsDNA donors with RNAiMAX. Different designs of dsDNA or control ssDNA (FIG. 18A) were tested. Genomic DNA was harvested from transfected cells after 48 hours and analyzed after amplification by ILLUMINA next generation sequencing.

### Results:

It was observed that precise insertions were introduced by KING editing using dsDNA donors with blunt, overhang or bridge designs. The absolute and relative precise editing efficiency were further improved by phosphorothioate bonds modification of donor oligos when using donors with blunt and overhang designs (FIG. 18B).

### Example 15. KING Editing with Cas9 (H840A) Nickase

In this Example, KING editing was tested with a Cas9 (H840A) nickase.

### Methods:

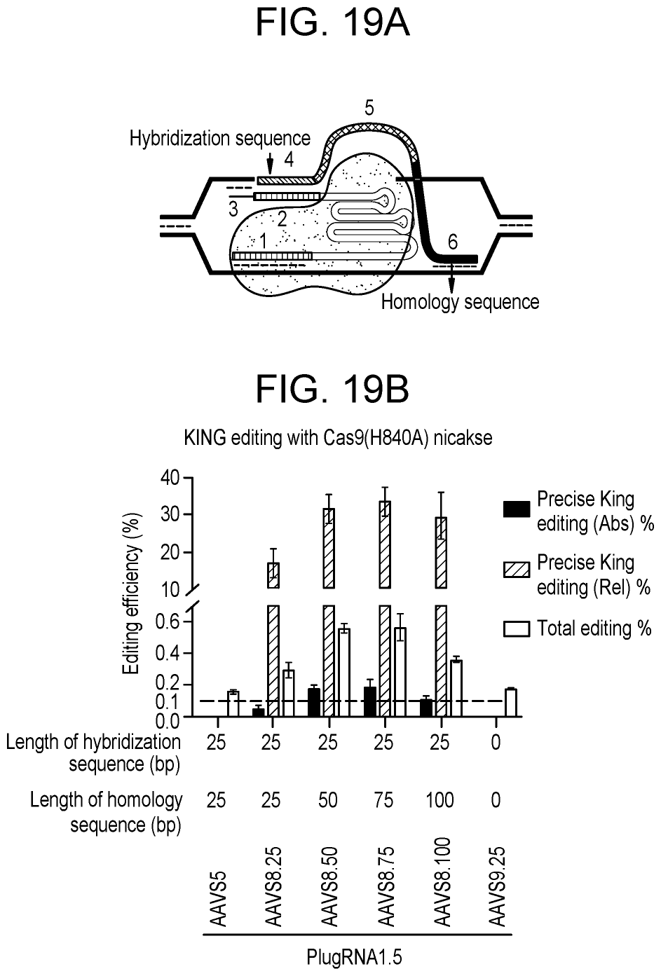

HEK293T cells were transfected with plasmid driving the expression of SpCas9 (H840A) nickase with FugeneHD. 24 hours later, plugRNAs and ssDNA donors were assembled using the following conditions: Denature at 95 C for 2 minutes and then ramp down to 4° C. at the rate of 0.1° C./s. The cells were transfected with 2 pmol of assembled plugRNAs and ssDNA donors with RNAiMAX. Different designs of ssDNA (FIG. 19A) were tested. Genomic DNA was harvested from transfected cells after 48 hours and analyzed after amplification by ILLUMINA next generation sequencing.

### Results:

It was observed that precise insertions were introduced by KING using SpCas9 (H840A) nickase. Relative precise editing efficiency of up to 36% was achieved when SpCas9 (H840A) nickase was used (FIG. 19B), which was higher than the precise editing efficiency achieved with SpCas9 nuclease (less than 5%).

### Example 16. KING Editing Upon Treatment with DNA-Dependent Protein Kinase (DNA-PK) Inhibitor

In this Example, KING editing was tested with a DNA-PK inhibitor. DNA-PK is part of the non-homologous end joining (NHEJ) repair pathway.

### Methods:

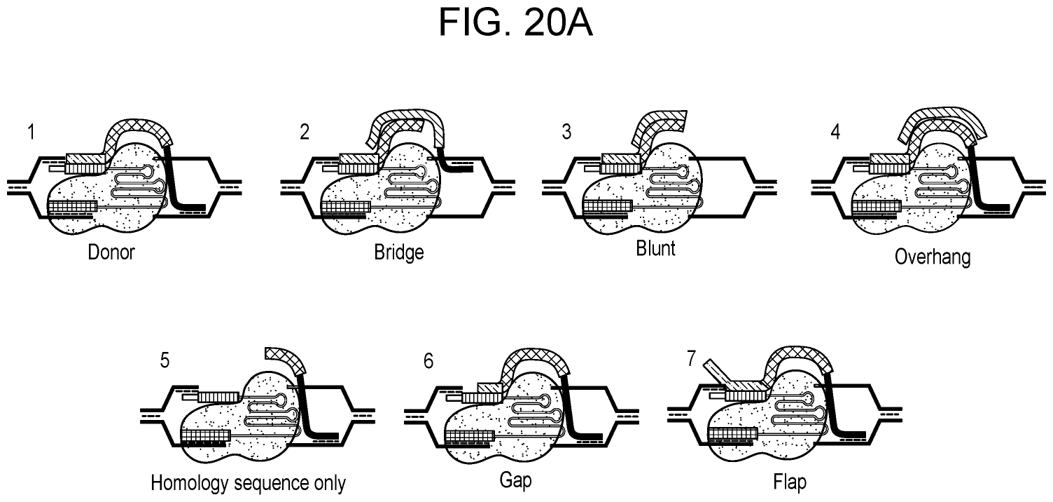

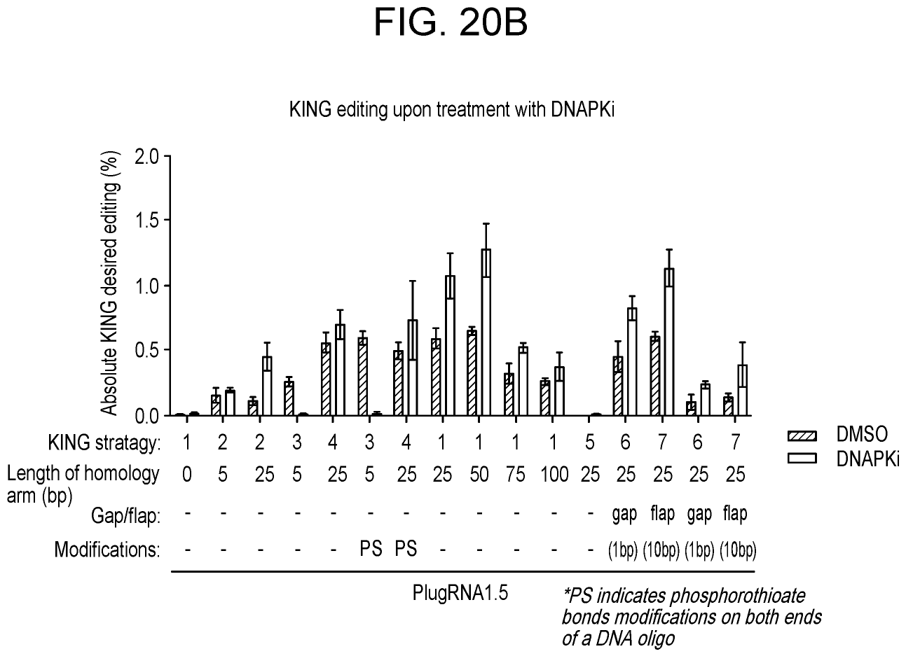

HEK293T cells were pretreated with 1 μM of DNAPK inhibitor (AZD7648) for 1 h prior the cells transfection. DMSO was used as a mock control. After 1 h, HEK293T cells were transfected with plasmid driving the expression of Cas9 and PEn with FugeneHD reagent. 24 hours later the cells were transfected with 2 pmol synthetic RNAs using RNAiMAX. Synthetic plugRNAs were annealed to the donor sequences by denaturing them for 2 minutes at 95° C. and then slowly cooled to 4° C. Several different plugRNAs and donors configurations were tested (see FIG. 20A).

Forty eight hours post-RNA transfection the cells were harvested, the AAVS1 locus was amplified by PCR and sequenced using Illumina platform. Data were analyzed by aligning the reads to a reference sequence and frequency of edits was analyzed by RIMA (PMID: 30032200).

Results show that Cas9 and PEn in combination with synthetic oligonucleotides performed KING insertions. The pretreatment with DNAPK inhibitor improved precise editing in HEK293T cells.

FIG. 20B represents shows the absolute desired editing percentage for different combination of transfected plugRNA:donor. Pretreatment with DNA-PK inhibitor improved absolute desire editing (light gray) in comparison to DMSO control (dark gray).

### Example 17. KING Editing in the Presence of Co-Expressed Ligases

In this Example, KING editing was performed in the presence of ligases or ligase-adaptor proteins (also referred to as “ligase recruitment proteins”).

### Methods:

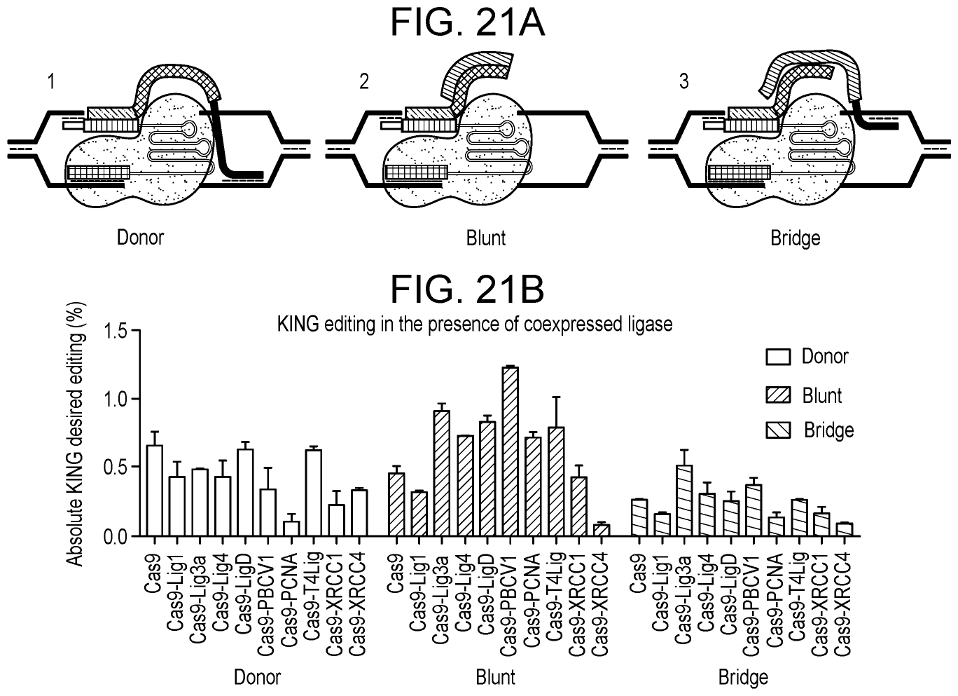

HEK293T cells were transfected with plasmid/plasmids driving the expression of Cas9 and ligase/ligase adaptors using FugeneHD reagent. A panel of three different donor configurations (FIG. 21A), each with nine different ligase conditions, were tested: ligases with bacterial origin (T4 DNA ligase, LigD and PBCV1); ligases with human origin (Ligl, Lig3a, Lig4); and human DNA repair proteins (XRCC1, XRCC4, PCNA). Cas9 and ligases were expressed from separate plasmids.

Forty eight hours post-RNA transfection the cells were harvested, and the AAVS1 locus was amplified by PCR and sequenced using Illumina platform. Data were analyzed by aligning the reads to a reference sequence, and the frequency of edits was analyzed by RIMA (PMID: 30032200).

FIG. 21B shows absolute desired editing percentage for the combinations of co-expressed Cas9 with different ligases. The results in show that Cas9 in the presence of ligases performed KING insertions.

### Example 18. Summary of Composition of Different Donors and their Performance in In Vitro Assays and in Cells

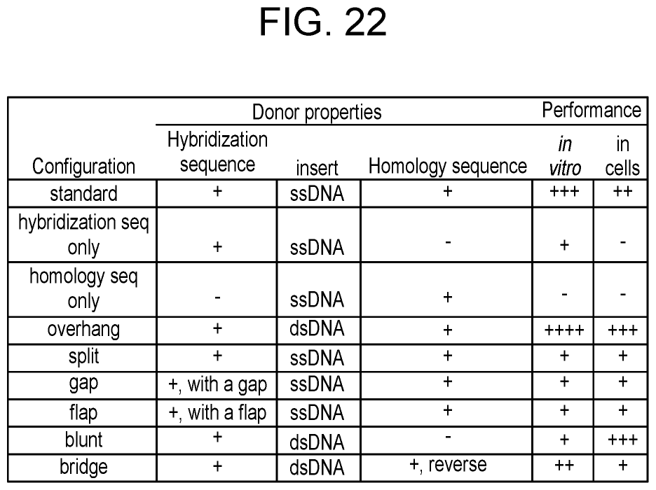

The Table in FIG. 22 summarizes the configurations of the plugRNA:donor complexes and editing efficiencies of each configuration as shown in the Examples herein. For example, “−” means no KING editing observed; “++++” means highly efficient editing.

### Example 19. Dual Hybridization Design

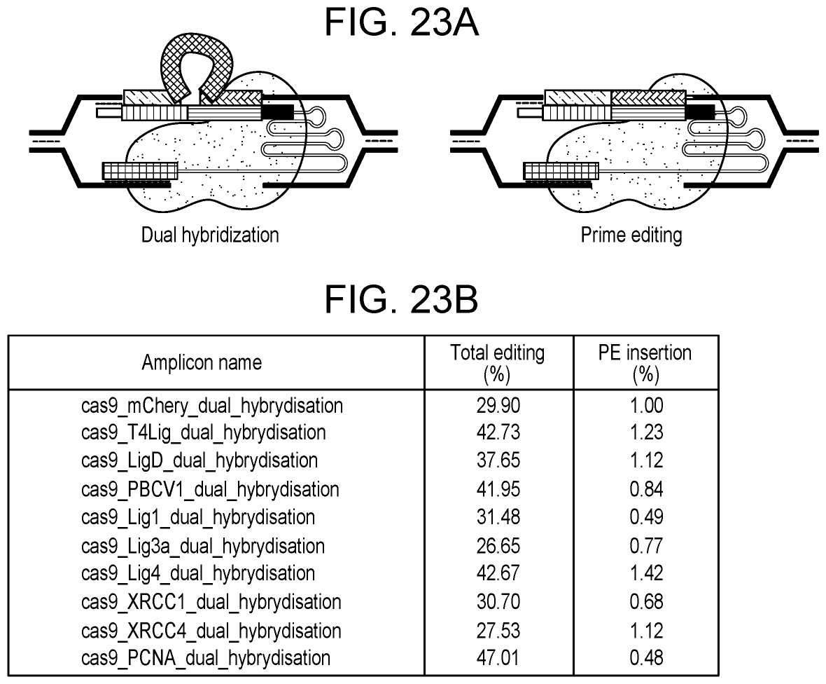

In this Example, a dual hybridization configuration (FIG. 23A) was tested.

### Methods:

HEK293T cells were transfected with plasmid driving the expression of Cas9 with FugeneHD. 24 hours later the cells were transfected with 2 pmol synthetic RNAs using RNAiMAX. Synthetic plugRNAs were annealed to the donor sequences by denaturing them for 2 minutes at 95° C. and then slowly cooled to 4° C.

Forty eight hours post-transfection the cells were harvested, and the AAVS1 locus was amplified by PCR and sequenced using Illumina platform. Data were analyzed by aligning the reads to a reference sequence, and the frequency of edits was analyzed by RIMA (PMID: 30032200).

### Results:

Prime editing insertions were observed. The results in FIG. 23B show the frequency of total editing and precise editing (PE) insertions.

What is claimed is:

1. A polynucleotide comprising (i) an RNA guide sequence; (ii) a Cas-binding region; and (iii) a DNA template sequence, wherein the DNA template sequence is at a 3′ end of the polynucleotide.

2. The polynucleotide of claim 1, wherein the DNA template sequence comprises a modified nucleotide, a non-B DNA structure, a DNA polymerase recruitment moiety, a DNA ligase recruitment moiety, or a combination thereof.

3. The polynucleotide of claim 2, wherein the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

4. The polynucleotide of claim 3, wherein the covalent linker comprises triethylene glycol (TEG).

5. The polynucleotide of claim 3, wherein the DNA lesion comprises 8-oxoguanine, thymine-glycol, or a combination thereof.

6. The polynucleotide of claim 3, wherein the DNA photoproduct comprises a cyclobutane pyrimidine dimer (CPD), a pyrimidine (6-4) pyrimidone photoproduct, or a combination thereof.

7. The polynucleotide of claim 3, wherein the modified deoxyribonucleoside comprises deoxyuridine.

8. The polynucleotide of claim 3, wherein the methylated nucleotide comprises 5-hydroxymethylcytosine, 5-methylcytosine, or a combination thereof.

9. The polynucleotide of claim 2, wherein the non-B DNA structure comprises a hairpin, a cruciform, Z-DNA, H-DNA (triplex DNA), G-quadruplex DNA (tetraplex DNA), slipped DNA, sticky DNA, or a combination thereof.

10. The polynucleotide of claim 2, wherein the DNA polymerase recruitment moiety comprises a DNA polymerase recruitment protein linked to the DNA template sequence.

11. The polynucleotide of claim 10, wherein the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

12. The polynucleotide of claim 2, wherein the DNA ligase recruitment moiety comprises a 5′ adenylation of the DNA template sequence.

13. A polynucleotide comprising: (i) an RNA guide sequence; (ii) a Cas-binding region; and (iii) a DNA template sequence, wherein the DNA template sequence is at a 3′ end of the polynucleotide, and wherein the DNA template sequence comprises a phosphorothioate bond.

14. The polynucleotide of any one of claims 1 to 13, wherein the DNA template sequence comprises a primer binding sequence and a sequence of interest.

15. The polynucleotide of any one of claims 1 to 14, wherein the Cas-binding region comprises RNA, or wherein the Cas-binding region comprises a combination of RNA and DNA.

16. The polynucleotide of any one of claims 1 to 15, wherein the Cas-binding region comprises a tracrRNA.

17. The polynucleotide of any one of claims 1 to 15, wherein the Cas-binding region is capable of hybridizing to a tracrRNA.

18. The polynucleotide of claim 16 or 17, wherein the tracrRNA is capable of binding to a Cas nuclease.

19. The polynucleotide of claim 18, wherein the Cas nuclease is Cas9 or Cas12a.

20. The polynucleotide of claim 18, wherein the Cas nuclease is a Type II-B Cas.

21. The polynucleotide of claim 16 or 17, wherein the tracrRNA is capable of binding to a Cas nickase.

22. The polynucleotide of claim 21, wherein the Cas nickase is a Cas9 nickase, a Cas12a nickase, or a Type II-B Cas nickase.

23. The polynucleotide of any one of claims 1 to 22, wherein the DNA template sequence is about 8 to about 10000 nucleotides in length.

24. The polynucleotide of any one of claims 14 to 23, wherein the primer binding sequence is about 4 to about 300 nucleotides in length.

25. The polynucleotide of any one of claims 14 to 24, wherein the sequence of interest is about 4 to about 100 nucleotides in length.

26. The polynucleotide of any one of claims 1 to 25, wherein the RNA guide sequence is about 15 to about 25 nucleotides in length.

27. The polynucleotide of any one of claims 1 to 26, further comprising a spacer positioned between the Cas-binding region and the DNA template sequence.

28. The polynucleotide of claim 27, wherein the spacer comprises a stop sequence for a DNA polymerase.

29. The polynucleotide of claim 28, wherein the spacer comprises more than one stop sequence.

30. The polynucleotide of claim 28 or 29, wherein the stop sequence comprises a secondary structure.

31. The polynucleotide of claim 30, wherein the secondary structure comprises a stem loop.

32. The polynucleotide of any one of claims 27 to 31, wherein the spacer is about 10 to about 200 nucleotides in length.

33. A cell comprising the polynucleotide of any one of claims 1 to 32.

34. A composition comprising:
a Cas nuclease or a Cas nickase; and
a polynucleotide comprising: (i) a guide sequence; (ii) a Cas-binding region; and (iii) a DNA template sequence, wherein the DNA template sequence is at a 3′ end of the polynucleotide.

35. A composition comprising:
a Cas nuclease or a Cas nickase;
a first polynucleotide comprising: (i) a guide sequence; (ii) a Cas-binding region; and (iii) a first hybridization region, wherein the first hybridization region is at a 3′ end of the first polynucleotide; and
a second polynucleotide comprising: (i) a second hybridization region that is complementary to the first hybridization region and (ii) a DNA template sequence.

36. A composition comprising:
a Cas nuclease or a Cas nickase;
a first polynucleotide comprising: (i) a guide sequence; and (ii) a first hybridization region, wherein the first hybridization region is at a 3′ end of the first polynucleotide; and
a second polynucleotide comprising: (i) a second hybridization region that is complementary to the first hybridization region; (ii) a Cas-binding region; and (iii) a DNA template sequence.

37. The composition of any one of claims 34 to 36, wherein the DNA template sequence comprises a primer binding sequence and a sequence of interest.

38. The composition of any one of claims 34 to 37, wherein the DNA template sequence comprises a modified nucleotide, a non-B DNA structure, a DNA polymerase recruitment moiety, a DNA ligase recruitment moiety, or a combination thereof.

39. The composition of claim 38, wherein the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

40. The composition of claim 39, wherein the modified nucleotide comprises a phosphorothioate bond.

41. The composition of claim 39, wherein the covalent linker comprises triethylene glycol (TEG).

42. The composition of claim 39, wherein the DNA lesion comprises 8-oxoguanine, thymine-glycol, or a combination thereof.

43. The composition of claim 39, wherein the DNA photoproduct comprises a cyclobutane pyrimidine dimer (CPD), a pyrimidine (6-4) pyrimidone photoproduct, or a combination thereof.

44. The composition of claim 39, wherein the deoxyribonucleoside comprises deoxyuridine.

45. The composition of claim 39, wherein the methylated nucleotide comprises 5-hydroxymethylcytosine, 5-methylcytosine, or a combination thereof.

46. The composition of claim 38, wherein the non-B DNA structure comprises a hairpin, a cruciform, Z-DNA, H-DNA (triplex DNA), G-quadruplex DNA (tetraplex DNA), slipped DNA, sticky DNA, or a combination thereof.

47. The composition of claim 38, wherein the DNA polymerase recruitment moiety comprises a DNA polymerase recruitment protein linked to the DNA template sequence.

48. The composition of claim 47, wherein the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

49. The composition of claim 38, wherein the DNA ligase recruitment moiety comprises a 5′ adenylation of the DNA template sequence.

50. The composition of any one of claims 34 to 49, wherein the guide sequence comprises RNA, or wherein the guide sequence comprises a combination of RNA and DNA.

51. The composition of any one of claims 34 to 50, wherein the Cas-binding region comprises RNA, or wherein the Cas-binding region comprises a combination of RNA and DNA.

52. The composition of any one of claims 34 to 51, wherein the Cas-binding region comprises a tracrRNA.

53. The composition of any one of claims 34 to 51, wherein the Cas-binding region is capable of hybridizing to a tracrRNA, and wherein the composition further comprises a tracrRNA.

54. The composition of claim 52 or 53, wherein the tracrRNA is capable of binding to the Cas nuclease or Cas nickase.

55. The composition of any one of claims 34 to 54, wherein the composition comprises a Cas nuclease.

56. The composition of claim 55, wherein the Cas nuclease is Cas9 or Cas12a.

57. The composition of claim 55, wherein the composition comprises a Cas nuclease, and wherein the Cas nuclease is a Type II-B Cas.

58. The composition of any one of claims 34 to 54, wherein the composition comprises a Cas nickase.

59. The composition of claim 58, wherein the Cas nickase is a Cas9 nickase, a Cas12a nickase, or a Type II-B Cas nickase.

60. The composition of any one of claims 34 to 59, wherein the DNA template sequence is about 8 to about 500 nucleotides in length.

61. The composition of any one of claims 34 to 60, wherein the primer binding sequence is about 4 to about 30 nucleotides in length.

62. The composition of any one of claims 34 to 61, wherein the sequence of interest is about 4 to about 100 nucleotides in length.

63. The composition of any one of claims 34 to 62, wherein the guide sequence is about 15 to about 25 nucleotides in length.

64. The composition of any one of claims 35 to 63, wherein the first hybridization region and the second hybridization region are RNA.

65. The composition of any one of claims 35 to 63, wherein the first hybridization region and the second hybridization region are single-stranded DNA.

66. The composition of any one of claims 35 to 63, wherein the first hybridization region is RNA and the second hybridization region is single-stranded DNA, or wherein the first hybridization region is single-stranded DNA and the second hybridization region is RNA.

67. The composition of any one of claims 36 to 66, wherein the first hybridization region is about 4 to about 5000 nucleotides in length.

68. The composition of any one of claims 36 to 66, wherein the second hybridization region is about 4 to about 5000 nucleotides in length.

69. The composition of any one of claim 34 or 37 to 63, wherein the polynucleotide further comprises a spacer positioned 5′ of the DNA template sequence.

70. The composition of any one of claims 35 to 68, wherein the second polynucleotide further comprises a spacer position 5′ of the DNA template sequence.

71. The composition of claim 69 or 70, wherein the spacer comprises a stop sequence for a DNA polymerase.

72. The composition of claim 71, wherein the spacer comprises more than one stop sequence.

73. The composition of claim 71 or 72, wherein the stop sequence comprises a secondary structure.

74. The composition of claim 73, wherein the secondary structure comprises a stem loop.

75. The composition of any one of claims 69 to 74, wherein the spacer is about 10 to about 200 nucleotides in length.

76. The composition of any one of claims 34 to 75, wherein the Cas nuclease or the Cas nickase is fused to a DNA polymerase recruitment protein.

77. The composition of claim 76, wherein the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

78. A composition comprising:
a Cas nuclease or a Cas nickase;
a first polynucleotide comprising: (i) a guide sequence; (ii) a Cas-binding region; (iii) a first hybridization region; and (iv) a primer-binding sequence, wherein the primer-binding sequence is at a 3′ end of the first polynucleotide; and
a second polynucleotide comprising: (i) a second hybridization region that is complementary to the first hybridization region; and (ii) a sequence of interest (SOI).

79. A composition comprising:
a Cas nuclease or a Cas nickase;
a first polynucleotide comprising: (i) a guide sequence; (ii) a Cas-binding region; and (iii) a first hybridization region, wherein the first hybridization region is at a 3′ end of the first polynucleotide;
a second polynucleotide comprising: (i) a second hybridization region that is complementary to the first hybridization region; (ii) a third hybridization region; and (iii) a primer-binding sequence, wherein the primer-binding sequence is at a 3′ end of the second polynucleotide; and
a third polynucleotide comprising: (i) a fourth hybridization region that is complementary to the third hybridization region; and (ii) a sequence of interest (SOI).

80. The composition of claim 78, wherein the first polynucleotide and/or the second polynucleotide comprises RNA, DNA, a modified nucleotide, or combination thereof.

81. The composition of claim 78 or 80, wherein the first polynucleotide and/or the second polynucleotide further comprises a homology sequence.

82. The composition of claim 81, wherein the first polynucleotide further comprises (v) the homology sequence, and the second polynucleotide comprises the second hybridization region, the SOI, and a further hybridization region that hybridizes with the first polynucleotide, wherein the second hybridization region and the further hybridization region flank the SOI.

83. The composition of claim 79, wherein any of the first polynucleotide, the second polynucleotide, and/or the third polynucleotide comprises RNA, DNA, a modified nucleotide, or combination thereof.

84. The composition of claim 79 or 83, wherein any of the first polynucleotide, the second polynucleotide, and/or the third polynucleotide further comprises a homology sequence.

85. The composition of claim 80 or 83, wherein the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

86. The composition of any one of claims 78 to 85, further comprising a SOI complement oligonucleotide that comprises a sequence complementary to the SOI.

87. The composition of claim 86, wherein the SOI complement oligonucleotide further comprises a homology sequence.

88. The composition of any one of claims 78 to 87, wherein the Cas nuclease or the Cas nickase is fused to a DNA polymerase, a DNA polymerase recruitment moiety, a DNA ligase, a DNA ligase recruitment moiety, a DNA binding protein, a DNA repair protein, or combination thereof.

89. The composition of claim 88, wherein the DNA polymerase recruitment moiety comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or combination thereof.

90. The composition of claim 88, wherein the DNA ligase comprises T4 DNA ligase, PCBV-1 DNA ligase, LigD, a Human Ligase protein, or combination thereof.

91. The composition of claim 88, wherein the DNA ligase recruitment moiety comprises a 5′ adenylation of the DNA template sequence.

92. The composition of claim 91, wherein the DNA binding protein comprises replication protein A (RPA) or a subunit thereof, a Single-Stranded DNA Binding Protein (SSBP), or combination thereof.

93. The composition of claim 88, wherein the DNA repair protein comprises Tyrosyl-DNA Phosphodiesterase 1 (TDP1), aprataxin, topoisomerase I, or combination thereof.

94. The composition of any one of claims 78 to 93, wherein the Cas-binding region comprises a tracrRNA.

95. The composition of any one of claims 78 to 93, wherein the Cas-binding region is capable of hybridizing to a tracrRNA, and wherein the composition further comprises a tracrRNA.

96. The composition of claim 94 or 95, wherein the tracrRNA is capable of binding to the Cas nuclease or Cas nickase.

97. The composition of any one of claims 78 to 96, wherein the composition comprises a Cas nuclease.

98. The composition of claim 97, wherein the Cas nuclease is Cas9 or Cas12a.

99. The composition of claim 97, wherein the Cas nuclease is a Type II-B Cas.

100. The composition of any one of claims 78 to 96, wherein the composition comprises a Cas nickase.

101. The composition of claim 100, wherein the Cas nickase is a Cas9 nickase, a Cas12a nickase, or a Type II-B Cas nickase.

102. The composition of any one of claims 78 to 101, wherein the sequence of interest is about 4 to about 100 nucleotides in length.

103. The composition of any one of claims 78 to 102, wherein the primer binding sequence is about 4 to about 30 nucleotides in length.

104. The composition of any one of claims 78 to 103, wherein the guide sequence is about 15 to about 25 nucleotides in length.

105. The composition of any one of claims 78 to 104, wherein the first hybridization region, and/or the second hybridization region are each about 4 to about 5000 nucleotides in length.

106. A cell comprising the composition of any one of claims 34 to 105.

107. The cell of claim 106, further comprising an exogenous DNA polymerase, an exogenous DNA ligase, or both.

108. A fusion protein comprising (i) a Cas nuclease or a Cas nickase; and (ii) a DNA polymerase recruitment protein, a DNA ligase, a DNA ligase recruitment moiety, a DNA binding protein, a DNA repair protein, or combination thereof.

109. The fusion protein of claim 108, wherein the fusion protein comprises a Cas nuclease.

110. The fusion protein of claim 109, wherein the Cas nuclease is Cas9 or Cas12a.

111. The fusion protein of claim 109, wherein the Cas nuclease is a Type II-B Cas.

112. The fusion protein of claim 108, wherein the fusion protein comprises a Cas nickase.

113. The fusion protein of claim 112, wherein the Cas nickase is a Cas9 nickase, a Cas12a nickase, or a Type II-B Cas nickase.

114. The fusion protein of any one of claims 108 to 113, wherein the DNA polymerase recruitment protein comprises a proliferating cell nuclear antigen (PCNA), a single-stranded DNA-binding protein (SSBP), a tumor necrosis factor, alpha-induced protein (TNFAIP), a polymerase delta-interacting protein (PolDIP), an X-ray repair cross-complementing protein (XRCC), a 5-Hydroxymethylcytosine Binding, ES Cell Specific (HMCES) protein, RAD1, RAD9, HUS1, or a combination thereof.

115. The fusion protein of any one of claims 108 to 113, wherein the DNA ligase comprises T4 DNA ligase, PCBV-1 DNA ligase, LigD, a Human Ligase protein, or combination thereof.

116. The fusion protein of any one of claims 108 to 113, wherein the DNA ligase recruitment moiety comprises a 5′ adenylation of the DNA template sequence.

117. The fusion protein of any one of claims 108 to 113, wherein the DNA binding protein comprises replication protein A (RPA) or a subunit thereof, a Single-Stranded DNA Binding Protein (SSBP), or combination thereof.

118. The fusion protein of any one of claims 108 to 113, wherein the DNA repair protein comprises Tyrosyl-DNA Phosphodiesterase 1 (TDP1), aprataxin, topoisomerase I, or combination thereof.

119. A polynucleotide encoding the fusion protein of any one of claims 108 to 118.

120. A vector comprising the polynucleotide of claim 119.

121. A cell comprising the fusion protein of any one of claims 108 to 118, the polynucleotide of claim 119, the vector of claim 120, or a combination thereof.

122. The cell of claim 121, further comprising the polynucleotide of any one of claims 1 to 32.

123. A method of providing a targeted insertion in a target DNA in a cell, comprising introducing the composition of any one of claims 34 to 77 into the cell, wherein the guide sequence is capable of hybridizing to the target DNA.

124. The method of claim 123, wherein the method does not comprise introducing a DNA polymerase into the cell.

125. The method of claim 124, wherein the Cas nuclease generates a double-stranded cleavage in the target DNA, and an endogenous DNA polymerase of the cell extends the DNA template sequence.

126. The method of claim 123, wherein the method further comprises introducing an exogenous DNA polymerase into the cell.

127. The method of claim 126, wherein the Cas nuclease generates a double-stranded cleavage in the target DNA, and the exogenous DNA polymerase extends the DNA template sequence.

128. The method of any one of claims 125 to 127, wherein the DNA template sequence comprises a primer binding sequence and a sequence of interest, and the DNA polymerase synthesizes a DNA strand complementary to the sequence of interest to form a double-stranded sequence comprising the sequence of interest.

129. The method of claim 128, wherein the double-stranded sequence is inserted into the cleaved target DNA.

130. The method of claim 129, wherein the double-stranded sequence is inserted into the cleaved target DNA by non-homologous end joining (NHEJ).

131. The method of claim 130, wherein the double-stranded sequence is inserted into the cleaved target DNA by a DNA ligase.

132. The method of claim 131, wherein the DNA ligase is an endogenous DNA ligase of the cell.

133. The method of claim 131, wherein the DNA ligase is an exogenous DNA ligase.

134. A method of providing a targeted insertion in a target DNA in a cell, comprising introducing the composition of any one of claims 78 to 105 into the cell, wherein the guide sequence is capable of hybridizing to the target DNA.

135. The method of claim 134, wherein the method does not comprise introducing a DNA polymerase or a DNA ligase into the cell.

136. The method of claim 135, wherein the Cas nuclease generates a double-stranded cleavage in the target DNA, and an endogenous DNA ligase of the cell ligates the sequence of interest to the cleaved target DNA.

137. The method of claim 134, wherein the method further comprises introducing an exogenous DNA ligase into the cell.

138. The method of claim 137, wherein the Cas nuclease generates a double-stranded cleavage in the target DNA, and the exogenous DNA ligase ligates the sequence of interest to the cleaved target DNA.

139. The method of any one of claims 134 to 138, wherein the sequence of interest is a single-stranded sequence that is converted to a double-stranded sequence by a DNA repair pathway of the cell.

140. A method of providing a targeted insertion in a target DNA, comprising contacting the composition of any one of claims 78 to 105 with the target DNA, wherein the guide sequence is capable of hybridizing to the target DNA.

141. The method of claim 140, further comprising contacting the target DNA with a DNA ligase.

142. The method of claim 141, wherein the Cas nuclease generates a double-stranded cleavage in the target DNA, and the DNA ligase ligates the sequence of interest to the cleaved target DNA.

143. The method of any one of claims 140 to 143, further comprising contacting the target DNA with a DNA polymerase, a protein in a DNA repair pathway, or combination thereof.

144. A composition comprising:
a Cas nuclease or a Cas nickase;
a first polynucleotide comprising: (i) a guide sequence; (ii) a Cas-binding region; (iii) a first hybridization region; and (iv) a primer-binding sequence, wherein the primer-binding sequence is at a 3′ end of the first polynucleotide; and
a second polynucleotide comprising: (i) a second hybridization region that is complementary to the first hybridization region; and (ii) a sequence of interest (SOI).
a third polynucleotide comprising: (i) a third hybridization region that is partially complementary to the second polynucleotide; and (ii) a sequence of interest (SOI).
